SEQUENCE LISTING (24 sequences)

SEQ ID NO: 1 - DNA, 1920 bp, Homo sapiens
atgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 2 - protein, 639 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 3 - DNA, 1947 bp, Oryctolagus cuniculus
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagcctgatgacgttgtttacaaacaatatgagagttatacagtaaccatcacctgccctttcacatatgcgactaggcaactaaagaagtccttttacaaggtggaagacggggaacttgtactcatcattgattccagcagtaaggaggcaaaggaccccaggtataagggcagaataacgttgcagatccaaagtaccacagcaaaagaattcacagtcaccatcaagcatttgcagctcaatgatgctgggcagtatgtctgccagagtggaagcgaccccactgctgaagaacagaacgttgacctccgactgctaactcctggtctgctctatggaaacctggggggctcggtgacctttgaatgtgccctggactctgaagacgcaaacgcggtagcatccttgcgccaggttaggggtggcaatgtggtcattgacagccaggggacaatagatccagccttcgagggcaggatcctgttcaccaaggctgagaacggccacttcagtgtagtgatcgcaggcctgaggaaggaagacacagggaactatctgtgcggagtccagtccaatggtcagtctggggatgggcccacccagcttcggcaactcttcgtcaatgaagagatcgacgtgtcccgcagcccccctgtgttgaagggctttccaggaggctccgtgaccatacgctgcccctacaacccgaagagaagcgacagccacctgcagctgtatctctgggaagggagtcaaacccgccatctgctggtggacagcggcgaggggctggttcagaaagactacacaggcaggctggccctgttcgaagagcctggcaatggcaccttctcagtcgtcctcaaccagctcactgccgaggatgaaggcttctactggtgtgtcagcgatgacgatgagtccctgacgacttcggtgaagctccagatcgttgacggagaaccaagccccacgatcgacaagttcactgctgtgcagggagagcctgttgagatcacctgccacttcccatgcaaatacttctcctccgagaagtactggtgcaagtggaatgaccatggctgcgaggacctgcccactaagctcagctccagcggcgaccttgtgaaatgcaacaacaacctggtcctcaccctgaccttggactcggtcagcgaagatgacgagggctggtactggtgtggcgcgaaagacgggcacgagtttgaagaggttgcggccgtcagggtggagctgacagagccagccaaggtagctgtcgagccagccaaggtacctgtcgacccagccaaggcagcccccgcgcctgctgaggagaaggccaaggcgcggtgcccagtgcccaggagaaggcagtggtacccattgtcaaggaagctgagaacaagttgtccagaacctcggctccttgcggaggaggtagcagtgcagagtgcggaagacccagccagtgggagcagagcgtctgtggatgccagcagtgcttcgggacaaagcgggagtgccaaaaggtga

SEQ ID NO: 4 - protein, 648 aa, Oryctolagus cuniculus
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKARCPVPRRRQWYPLSRKLRTSCPEPRLLAEEVAVQSAEDPASGSRASVDASSASGQSGSAKR

SEQ ID NO: 5 - DNA, 1935 bp, Artificial Sequence
  CHIMERA (R/HpIgR)
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 6 - protein, 644 aa, Artificial Sequence
  CHIMERA (R/HpIgR)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPELLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 7 - DNA, 411 bp, Artificial Sequence
  CHIMERA (R/HpIgR)
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagag

SEQ ID NO: 8 - protein, 137 aa, Artificial Sequence
  CHIMERA (R/HpIgR)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPE

SEQ ID NO: 9 - DNA, 1524 bp, Artificial Sequence
  CHIMERA (R/HpIgR)
ctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 10 - protein, 507 aa, Artificial Sequence
  CHIMERA (R/HpIgR)
LLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 11 - DNA, 2031 bp, Artificial Sequence
  HUMAN pIgR-CD4 cyto (HpIgR-cyto)
atgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 12 - protein, 676 aa, Artificial Sequence
  HUMAN pIgR-CD4 cyto (HpIgR-cyto)
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

SEQ ID NO: 13 - DNA, 2058 bp, Artificial Sequence
  RABBIT pIgR-CD4 cyto (RpIgR-cyto)
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagcctgatgacgttgtttacaaacaatatgagagttatacagtaaccatcacctgccctttcacatatgcgactaggcaactaaagaagtccttttacaaggtggaagacggggaacttgtactcatcattgattccagcagtaaggaggcaaaggaccccaggtataagggcagaataacgttgcagatccaaagtaccacagcaaaagaattcacagtcaccatcaagcatttgcagctcaatgatgctgggcagtatgtctgccagagtggaagcgaccccactgctgaagaacagaacgttgacctccgactgctaactcctggtctgctctatggaaacctggggggctcggtgacctttgaatgtgccctggactctgaagacgcaaacgcggtagcatccttgcgccaggttaggggtggcaatgtggtcattgacagccaggggacaatagatccagccttcgagggcaggatcctgttcaccaaggctgagaacggccacttcagtgtagtgatcgcaggcctgaggaaggaagacacagggaactatctgtgcggagtccagtccaatggtcagtctggggatgggcccacccagcttcggcaactcttcgtcaatgaagagatcgacgtgtcccgcagcccccctgtgttgaagggctttccaggaggctccgtgaccatacgctgcccctacaacccgaagagaagcgacagccacctgcagctgtatctctgggaagggagtcaaacccgccatctgctggtggacagcggcgaggggctggttcagaaagactacacaggcaggctggccctgttcgaagagcctggcaatggcaccttctcagtcgtcctcaaccagctcactgccgaggatgaaggcttctactggtgtgtcagcgatgacgatgagtccctgacgacttcggtgaagctccagatcgttgacggagaaccaagccccacgatcgacaagttcactgctgtgcagggagagcctgttgagatcacctgccacttcccatgcaaatacttctcctccgagaagtactggtgcaagtggaatgaccatggctgcgaggacctgcccactaagctcagctccagcggcgaccttgtgaaatgcaacaacaacctggtcctcaccctgaccttggactcggtcagcgaagatgacgagggctggtactggtgtggcgcgaaagacgggcacgagtttgaagaggttgcggccgtcagggtggagctgacagagccagccaaggtagctgtcgagccagccaaggtacctgtcgacccagccaaggcagcccccgcgcctgctgaggagaaggccaaggcgcggtgcccagtgcccaggagaaggcagtggtacccattgtcaaggaagctgagaacaagttgtccagaacctcggctccttgcggaggaggtagcagtgcagagtgcggaagacccagccagtgggagcagagcgtctgtggatgccagcagtgcttcgggacaaagcgggagtgccaaaaggtgccggcaccgaaggcgccaagcagagcggatgtctcagatcaagagactcctcagtgagaagaagacctgccagtgccctcaccggtttcagaagacatgtagccccatttga

SEQ ID NO: 14 - protein, 685 aa, Artificial Sequence
  RABBIT pIgR-CD4 cyto (RpIgR-cyto)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKARCPVPRRRQWYPLSRKLRTSCPEPRLLAEEVAVQSAEDPASGSRASVDASSASGQSGSAKRCRHRRRQAERMSQIKRLLSEKKTCQCPHRFQKTCSPI

SEQ ID NO: 15 - DNA, 2046 bp, Artificial Sequence
  CHIMERA-CD4 cyto (R/HpIgR-cyto)
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 16 - protein, 681 aa, Artificial Sequence
  CHIMERA-CD4 cyto (R/HpIgR-cyto)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPELLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

SEQ ID NO: 17 - DNA, 411 bp, Artificial Sequence; identical to SEQ ID NO: 7
  CHIMERA-CD4 cyto (R/HpIgR-cyto)

SEQ ID NO: 18 - protein, 137 aa, Artificial Sequence; identical to SEQ ID NO: 8
  CHIMERA-CD4 cyto (R/HpIgR-cyto)

SEQ ID NO: 19 - DNA, 1635 bp, Artificial Sequence
  CHIMERA-CD4 cyto (R/HpIgR-cyto)
ctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 20 - protein, 544 aa, Artificial Sequence
  CHIMERA-CD4 cyto (R/HpIgR-cyto)
LLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

SEQ ID NO: 21 - protein, 499 aa, Artificial
  CB6IgA1
  misc_feature (495)..(495): Xaa can be any naturally occurring amino acid
ASEVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCYXGPSR

SEQ ID NO: 22 - protein, 486 aa, Artificial
  CB6IgA2
  misc_feature (482)..(482): Xaa can be any naturally occurring amino acid
ASEVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCYXSRGP

SEQ ID NO: 23 - protein, 219 aa, Artificial
  CB6 kappa chain
ASDIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECS

SEQ ID NO: 24 - protein, 145 aa, Artificial
  J chain
SRVAGITAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

(19) United States
(12) Patent Application Publication
(10) Pub. No.: US 2024/0288440 A1
(43) Pub. Date: Aug. 29, 2024

(54) METHODS AND COMPOSITIONS BASED ON LONGITUDINAL STUDIES

(71) Applicant: Macfarlane Burnet Institute for Medical Research and Public Health, Melbourne (AU)

(72) Inventors: David Anderson, Melbourne (AU); Heidi Drummer, Melbourne (AU); Purnima Bhat, Melbourne (AU); Huy Van, Melbourne (AU)

(21) Appl. No.: 18/247,984

(22) PCT Filed: Oct. 6, 2021
(86) PCT No.: PCT/AU2021/051167

§ 371 Date: Apr. 5, 2023

(30) Foreign Application Priority Data
Oct. 6, 2020 (AU) 2020903616
Dec. 11, 2020 (AU) 2020904633
Jun. 28, 2021 (AU) 2021901958

(51) Int. Cl. G01N 33/68; A61K 39/00; A61K 39/42; C07K 16/10; G01N 33/569

(52) U.S. Cl. CPC G01N 33/6854; A61K 39/42; C07K 16/1003; G01N 33/56983; A61K 2039/505; C07K 2317/35; C07K 2317/76; G01N 2333/165; G01N 2800/12; G01N 2800/52

(57) ABSTRACT

The present application relates to infectious diseases, pathogenic organisms or pathogenic antigens, and the immune responses that are the body's first line of defense thereto. The application enables medical protocols and products inter alia for treating or preventing or limiting the dissemination of an infectious disease. Methods and compositions are disclosed which employ dIgA for assessing functional immune responses to a pathogen, and in prophylactic or therapeutic compositions. In particular embodiments, the methods and compositions enhance the armamentarium for those charged with managing infectious diseases and populations exposed to highly transmissible and potentially debilitating or fatal pathogens such as those causing epidemics. One particular infectious disease is COVID-19 caused by the virus SARS-COV-2.

### FIELD

The present application relates to infectious diseases, pathogenic organisms or pathogenic antigens, and the immune responses that are the body's first line of defense thereto. The application enables medical protocols and products inter alia for treating or preventing or limiting the dissemination of an infectious disease. Methods and compositions are disclosed which employ dIgA for assessing functional immune responses to a pathogen, and in prophylactic or therapeutic compositions. In particular embodiments, the methods and compositions enhance the armamentarium for those charged with managing infectious diseases and populations exposed to highly transmissible and potentially debilitating or fatal pathogens such as those causing epidemics. One particular infectious disease is COVID-19 caused by the virus SARS-COV-2.

### BACKGROUND

Bibliographic details of references in the subject specification are also listed at the end of the specification.

Reference to any prior art in this specification is not, and should not be taken as, acknowledgement or any form of suggestion that this prior art forms part of the common general knowledge in any country.

Australian provisional patent application no 2020903616 filed 6 Oct. 2020, Australian provisional patent application no 2020904633 filed 11 Dec. 2020 and Australian provisional patent application no 2021901958 filed 28 Jun. 2021 are incorporated herein in full by reference.

Immune-based therapeutics provide some of the most widely used pharmaceutical and veterinary agents. Vaccine technology has been particularly successful, typically using exposed components of the pathogen's invasive machinery to engender a recallable immune response that modulates the development of infections. However, there are still many infectious diseases including, of course, emerging pathogens for which there are no effective therapeutics or prophylactic vaccines.

Many infectious diseases exist resulting from various classes of organism, viruses, parasites, bacteria, fungi etc. Of these, new viruses seem to provoke serious epidemics and pandemics.

Efforts in the face of a new virus pandemic focus on minimizing viral transmission through social distancing and contact tracing of identified cases. In the case of COVID-19, case identification relies primarily on diagnosis through detection of viral RNA through reverse transcription polymerase chain reaction (RT-PCR) or other nucleic acid amplification methods, or of viral antigen through immunochemical methods. There is a great need for reliable, sensitive and facile methods for detecting recent SARS-COV-2 infection useful for screening and contact tracing, for measuring immune protection among SARS-COV-2 infected subject, methods for determining the potential efficacy of therapeutic and prophylactic measures, including convalescent plasma therapy and vaccine efficacy.

Antibody responses to SARS-COV-2 are thought to be important for both diagnosis, epidemiological and immunological insight and contact tracing or other population screens, and in assessing immunity to infection, whether from natural infection or passive immunotherapy or active immunization.

For diagnosis, it is important to be able to distinguish recent infections (e.g. less than 1, 2 or 3 months) from more distant infections, especially for the purpose of diagnosis of acute disease (a current infection), contact tracing, workplace safety and epidemiology. IgM antibodies are typically used to detect relatively recent infections, however IgM assays typically have high rates of false-positive results (between 1-4%) which makes them unsuitable for infections with low prevalence such as SARS-COV-2 in most settings. Secretory IgA (SIgA) is recognised as the main mediator of effective immune responses at mucosal pathogen entry points.

Various assays measure serum IgG antibody binding to SARS-COV-2 spike protein or the receptor binding domain (RBD) of the spike protein. The RBD recognizes human receptor angiotensin converting enzyme 2 (ACE2) which is expressed by alveolar epithelial cells and facilitates virus entry of host cells.

In the circulation, IgA is the second most abundant Ig class, its concentration (about 2 mg/ml) being surpassed only by that of IgG (about 12 mg/ml). Considering the distribution in various body fluids of the major Ig isotypes and their catabolic rates, IgA is clearly synthesized in quantities (66 mg/kg body weight/day) that exceed by far the combined daily synthesis of all other isotypes. However, most of this newly synthesized IgA is secreted at mucosal surfaces as SIgA. In man, only a very small proportion of the dimeric or polymeric dIgA comprising IgA monomers plus a J-chain is found circulating in the blood/plasma at any time, because of the rapid export as SIgA. As most IgA detection reagents are antibodies that equally recognize epitopes on monomeric and dimeric IgA, the specific role of dIgA has not been well studied. Similarly, J-chain detection reagents will recognize both polymeric IgA as well as pentameric and polymeric IgM which also contain the J-chain.

Wang et. al. (Sci. Transl. Med. 10.1126/scitranslmed.abf1555 (2020)) disclose neutralization assays showing dIgA is more potent than the corresponding mIgA consistent with the presence of neutralising dIgA at mucosal surfaces and mIgA in the circulation. Wang et. al. incorrectly assert that mIgA and dIgA are produced by the same cells suggesting that the magnitude of mIgA (or total IgA) is reflective of the dIgA response, and therefore mIgA or total IgA could be measured to determine the level of dIgA. However, there is strong evidence that dIgA and mIgA are instead produced by discrete populations of B-cells, and that it is necessary to measure dIgA directly to determine the level of dIgA.

The polymeric Ig receptor (pIgR) transports dIgA across the mucosal epithelium. For example, dIgA at the sub-mucosal surface binds pIgR at the basolateral surface of the epithelial cell layer. The complex is internalised and undergoes vesicular transport across the cell. pIgR is cleaved to release secretory component (SC), which becomes disulphide-bonded to the dimeric IgA. At the apical surface, SIgA is released. SIgA binds to and neutralizes pathogens such as bacterial and viral pathogens at the lumenal surface such as the lungs or the gut. Some pathogens may gain access to the lamina propria underlying the epithelium and can be bound by dimeric IgA. The dimeric IgA-pathogen complex binds to pIgR and the pathogen is carried out across the epithelium and released back out into the lumen. Some pathogens can be intersected by dimeric IgA during transit across the epithelial cells. Here, the pathogen is ejected upon release of SIgA at the mucosal surface, but may also be neutralized within the cell. Dimeric IgA can also mediate clearance mechanisms against pathogens through engaging phagocytosis.

Antibody therapies for COVID-19 currently being tested, including monoclonal antibodies are typically IgG. Similarly, current proposed methods for screening convalescent plasma require screening for high circulatory virus-specific IgG levels as a positive selection measure, and intravenous polyclonal immunoglobulin compositions are traditionally at least 90% pure IgG (Afonso & Joao, Biomolecules, 2016, 6, 15). During apheresis, in addition to neutralizing antibodies, other proteins such as anti-inflammatory cytokines, clotting factors, natural antibodies, defensins, pentraxins and other undefined proteins are obtained from donors. In this sense, transfusion of convalescent plasma to infected patients may provide further benefits such as immunomodulation via amelioration of severe inflammatory response.

### SUMMARY

The term “and/or”, e.g., “X and/or Y” shall be understood to mean either “X and Y” or “X or Y” and shall be taken to provide explicit support for both meanings or for either meaning. As used herein, the term “about”, unless stated to the contrary, refers to +/−10%, more preferably +/−5%, even more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein, the singular form “a”, “an” and “the” include singular and plural references unless the context indicates otherwise. Each embodiment in this specification is to be applied mutatis mutandis to every other embodiment unless expressly stated otherwise.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. Sequence identifiers are described in Table 2. A sequence listing is provided after the claims.

Immune assays for assessing immune responses can measure different antibody isotypes (IgG, IgA, IgM) or total antibody or total virus-neutralizing antibody (using direct or surrogates of neutralization such as inhibition of RBD-ACE2 interaction), but all these assays ignore the fact that SARS-COV-2 infects mucosa; tissues (for example, lung and respiratory epithelium, and also the gut epithelium) where these antibodies are not found in large amounts. Instead, the major form of antibody in these tissues is secretory IgA (SIgA), which is derived from dimeric IgA (dIgA) in the plasma by transport via the polymeric Ig receptor (pIgR, or SC). Notably, in humans and higher primates only around 10% of IgA in the plasma is dIgA, with 90% being monomeric IgA that is not related to mucosal immune responses. Therefore, measurement of IgA (while failing to distinguish between monomeric and dimeric IgA) in humans does not necessarily reflect the mucosal response. In mice, which are commonly studied, the IgA is nearly all dIgA, and it is commonly assumed this is also true in humans. Mucosal antibody responses (e.g. SIgA in humans) can be measured directly in samples such as saliva, nasal washings, lung washings, gut samples or faeces, but these are very inconsistent due to sampling errors and dilution and have not proven useful for monitoring vaccine responses in particular.

International Patent Publication no. WO 2014/071456 in the name of Macfarlane Burnet Institute for Medical Research and Public Health describes a chimeric form of SC, called CSC, which allows for the very specific detection of antigen specific dIgA in the presence of the large excess of IgA and other antibodies. WO 2014/071456 discloses the dIgA response was early and transient in subjects with infections including HCV and HAV allowing early detection of infection. Total (monomeric) IgA persisted in most HCV positive patients (Mohd Hanafiah et al, 2018). Therefore, dIgA but not monomeric IgA could be used to diagnose acute infections. A specificity improvement was the only difference expected using the dIgA response compared to the IgM response. Thus, for example, WO 2014/071456 proposes measuring antigen specific dIgA and IgM to diagnose an acute (current) infection with a pathogen.

In the work leading up to the present application, preliminary longitudinal studies revealed that antigen specific dIgA levels in the blood of subjects infected with SARS-COV-2 show substantial levels early in infection. Immuno-assays have been developed, including laboratory based (eg. ELISA-based formats and bead-based assays) and point of care (eg. lateral flow immunochromatographic assays), that employ a pIgR-based reagent (such as CSC) to provide for the specific detection of dIgA from among the complex mixture of molecules found in biological samples such as blood. In one embodiment, lateral flow strips are used in conjunction with an instrument reader to quantify pathogen specific polymeric IgA levels. Further, these assays show a strong correlation between the level or presence of dIgA and the time since diagnosis or symptom onset and dIgA levels drop precipitously and reliably at about 2-3 months after infection. In one embodiment, the assays and kits test for antigen specific dIgA and the same antigen specific either total Ig or IgG. The information of dIgA level, or the combined information of dIgA and total Ig or IgG antigen specific immunoglobulin provides information about the time since infection and the magnitude of the mucosal immune response generated by that subject to the pathogen. Specifically, as IgG levels are not detectable in the first say about 12 to 14 days after symptoms commence and start increasing about the time dIgA levels are declining, the addition of the IgG response information distinguishes between the lower dIgA levels seen both at the start and the end of the dIgA trajectory (dIgA levels over time).

Accordingly, by testing for both dIgA and total antibody/IgG the assay/kit differentiates a very recent infection (less than around 12-14 days since symptom onset) in the absence of significant total antibody, versus the same level of dIgA being indicative of less recent infection (more than around 12-14 days and most likely more than around 45 days since symptom onset) when in the presence of higher levels of total antibody/IgG reactivity.

Accordingly, by testing for both dIgA and IgG the assay/kit differentiates a very recent infection (less than around 12-14 days since symptom onset) in the absence of significant total antibody, versus the same level of dIgA being indicative of less recent infection (more than around 12-14 days and most likely more than around 45 days since symptom onset) when in the presence of higher levels of IgG reactivity.

Surprisingly, the level of IgM is not informative in this context and information regarding specifically IgM is not determined in the present assay/kit. In one embodiment, total Ig/IgG is not included in the assay/kit and, instead the dIgA levels are determined on more than one occasion (for example spaced over several days or weeks) to allow a user to estimate the time since infection.

In another embodiment, total Ig/IgG is not included in the assay/kit and, instead presence of a level or defined level of dIgA is used to estimate a maximum time since infection (for example, less than three months).

In another embodiment, where available, data from the subject concerning when they had symptoms would also facilitate this distinction. The predictable early rapid rise and rapid fall of circulatory dIgA levels seen in significant numbers determined herein indicates a functional mucosal immune response to the pathogen which may be a CoV pathogen or any pathogen or pathogenic antigens where a mucosal immune response is generated.

Accordingly, the present invention though exemplified with respect to CoV is not so limited. As the skilled person will appreciate the exact nature of the mucosal response will vary between pathogens and between pathogen antigens, such as the infection rate, the median time from infection to symptoms or rise in dIgA levels, the median time to decline in dIgA levels and/or rise in IgG levels.

Not all subjects infected with a pathogen show symptoms. In the context of contact tracing and immune surveillance the detection of circulating dIgA during early infection can be very informative. In one embodiment, the level of IgM is not determined because no correlation between the level of IgM over time was detected.

Applications of the present methods and kits include, triage testing, confirmation of triage testing, diagnosis and differential diagnosis of symptomatic individuals, determination of previous exposure, population surveillance, environmental testing, health checks and surveys, and screening of non-symptomatic individuals.

In addition, the inventors determined that this reliable trajectory provides a number of further distinct advantages over the prior art. While the level of dIgA are not of course absolute, and some subjects remain dIgA positive for longer or shorter periods, the shape of the curve (dIgA levels against time) or the shape transformed into a reference value, allow the skilled person to estimate with confidence that someone with dIgA say with an OD above 1 was infected within a particular period and additionally estimate the date of infection/from the shape of the curve. In one study, subjects were not showing threshold levels of dIgA before 4 days but were all above a threshold by 10 days to 30 days from an early start date. A decreasing proportion of subjects was positive from 16 to 66 days. This reliable trajectory allows both the time since infection and the magnitude of the dIgA response to be much more precisely estimated. This previously unappreciated relationship, not displayed by IgG, IgA or IgM, in itself increases the armamentarium for those charged with managing infectious diseases. This provides for methods of contact tracing and population identification which are particularly valuable where the infection can by asymptomatic as with SARS-COV-2. Detection of recent asymptomatic infections especially during contact tracing is a major advantage of the present screening methods.

Furthermore, the inventors reasoned that the rapid disappearance of circulating antigen specific dIgA from the plasma indicates that B-cells have subsequently migrated to mucosal sites and that the residual dIgA antibody produced by plasma cells at the lamina propria is being rapidly excreted as SIgA at mucosal surfaces. Thus, detection of circulatory dIgA can now be used to screen populations for response to vaccine administration or seroconversion and as a marker for vaccine efficacy especially for induction of SIgA responses which largely effect the mucosal immune response. Further studies described herein show that, surprisingly, neutralizing antibodies displayed their highest levels relatively early i.e., 4 to 5 weeks after infection, and a significant proportion of circulating neutralizing antibody is dIgA at this early stage (1 to 3 months after infection). Accordingly, it is determined that the presence and level of circulating dIgA at this early stage is an accurate marker of the magnitude of the effective mucosal immune response that a subject is able to generate and thus a marker for vaccine efficacy.

In one embodiment, the level of circulating anti-RBD dIgA closely correlates with the magnitude and presence of a neutralizing SIgA at the mucosa. In some embodiments this is measured with a neutralizing antigen, such as Spike or a part thereof comprising relevant epitopes. In other embodiment, it is measured using a non-neutralizing antigen which nevertheless acts as a marker for a neutralizing mucosal immune response. For example, in one embodiment in the case of CoV, another antigen such as (eg. Nucleocapsid) provides a useful level of correlation, as a marker of an overall strong dIgA/mucosal response. In one embodiment, the mucosal surface of a subject includes respiratory, gut, reproductive mucosal surfaces.

In one embodiment, the AUC for plasma dIgA allows the user to measure or estimate the magnitude of the SIgA response i.e., the mucosal immune response. For example, without being bound to a particular mode of action, it is proposed herein that a dIgA-producing B cell has a residence time of say 7 days before settling down in the lamina propria, after which the dIgA produced in the circulation rapidly disappears and its newly formed dIgA is exported directly as SIgA (as per FIG. 24). Accordingly, the longer dIgA is detected in plasma, the longer the subject has been making dIgA-producing B cells that will have accumulated throughout that time in the lamina propria, ie the AUC reflects the total response. The presence of antigen-specific SIgA at the mucosa can be detected using the subject assays/kits with a biological sample derived from a muscosal niche eg. saliva.

Furthermore, by identifying dIgA as highly neutralizing with levels that correlate with the effectiveness of the immune response to a pathogen, this provides inter alia for improved methods of stratifying convalescent human subjects for prioritisation as a blood or plasma donor for convalescent plasma therapy or for preparation of human convalescent immunoglobulins. Although the term “plasma” and “blood” are used for convenience, this is not limiting and any suitable blood sample, fraction, product or derivative comprising antibodies may be employed. In one embodiment, the method comprises a timely assessment of the level or presence of antigen specific dIgA, such as RBD or Spike antigen specific dIgA, in a blood sample from the subject who has been infected with the pathogen and typically no longer poses an infection risk. Samples are stratified as, for example, no dIgA, low dIgA, medium dIgA or high dIgA based typically on population studies. Subjects identified with high or medium levels of antigen specific dIgA may be immediately prioritised as blood/plasma donors. As determined herein dIgA levels decline rapidly at about 2-3 months from infection mark and thus blood/plasma must be donated in a timely fashion. As determined herein, the herein disclosed rapid device is able to clearly distinguish between subjects who have no, medium or high neutralizing pathogen specific dIgA levels.

For the avoidance of doubt, neutralizing antigens are directly able as determined herein to detect a substantial early circulating dIgA response in subjects infected with the pathogen, and the dIgA response is substantially neutralizing. Further, non-neutralizing antigens may be used to detect and quantify the dIgA response and therefore provide a surrogate marker for the neutralizing antibody response. Accordingly, in one embodiment, where the specification describes assays employing neutralizing antigens it must be noted that non-neutralizing antigens are also a surrogate marker for the subjects ability to mount a neutralizing mucosal immune response. The detection of circulating (blood/plasma/serum) dIgA is found to be a reliable, quantitative and facile method for detecting a functional mucosal immune response to a pathogen or pathogenic antigen.

In one particular embodiment, levels are compared with a pre-determined plot of dIgA level against time or reference stratum values representing same, to establish a time since infection or the magnitude of the dIgA response as described further herein. In one embodiment, the identification of a level of pathogen specific dIgA or the magnitude of the dIgA response indicates seroconversion to dIgA by the subject and the production by the subject of pathogen specific functional antibodies able to effect a functional mucosal response. This, therefore, determines inter alia the suitability of the subject for prioritisation as a blood or plasma donor for therapeutic or prophylactic convalescent plasma administration against the pathogen, or for the production of compositions comprising high levels of antigen specific polyclonal dIgA. In some embodiments, neutralizing assays are also conducted with plasma to consolidate the results. The provision of circulating antibodies from plasma or serum with high levels of neutralizing dIgA will have the added advantage of being specifically and efficiently secreted into mucosal surfaces via pIgR, whereas neutralizing IgG or other antibodies will not be secreted efficiently. In one embodiment, administration is systemic, via for example, by direct intravenous, subcutaneous or intramuscular delivery.

There are many useful applications for the present disclosure. In another embodiment, plasma or serum that is identified as having medium or high levels of dIgA is processed to enrich for dIgA. Synthetic plasma or mixed or other blood products or therapeutic or prophylactic compositions are processed to provide dIgA in preference to other isotypes to provide a surrogate of an efficient mucosal immune response to an invading pathogen. Further still, dIgA may be purified from blood from early convalescent or immunised subjects and processed to provide physiological or pharmacological antibody compositions comprising dIgA as an active ingredient sufficient to provide an effective mucosal immune response at a mucosal surface. In one embodiment, pIgR based reagents such as CSC or rabbit pIgR are used for capturing dIgA at solid or semi-solid surfaces (affinity matrix, agarose beads etc). Recombinant or cloned dIgA antibody products for therapy or prophylaxis are proposed. Thus, total dIgA or pathogen or antigen or venom specific dIgA antibodies are selected for use in the manufacture of medicaments to treat or prevent pathogenic infections at mucosal surfaces, such as COVID-19, or to remove pathogenic antigens or venoms from the circulation via secretion at mucosal surfaces. Convalescent blood or blood products may also provide B-cells for antibody isolation and cloning.

In another embodiment, the application enables a method of identifying a subject or a blood sample from a subject as a responder or non responder to a vaccine, or quantifying the magnitude of the mucosal immune response by measuring the level of the circulating pathogen specific dIgA antibody in the subject at a suitable or known time from vaccination.

In one embodiment, the application enables a method for identifying or stratifying a convalescent human subject for prioritisation as a blood or plasma donor for convalescent plasma therapy or purification of immunoglobulin, said method comprising assessing the level or presence of pathogen specific dIgA in a blood sample from the subject who has been infected with the pathogen, wherein the identification of a level of pathogen specific dIgA in the blood sample indicates the production by the subject of pathogen specific functional (e.g. neutralizing) antibodies and the suitability of the subject for prioritisation as a blood or plasma donor for therapeutic or prophylactic convalescent plasma or immunoglobulin administration against the pathogen. Plasma cells expressing pathogen specific dIgA directly derived from the convalescent subject may be used to purify the dIgA immunoglobulin to administration to the subject. Administration may be into the circulatory system or to directly to a mucosal surface.

In one embodiment, the methods disclosed herein for detecting circulating pathogen specific dIgA use a non-antibody dIgA binding agent. In one exemplified embodiment the dIgA binding agent is derived from human pIgR and comprises a heterologous domain 1 that binds to dIgA and substantially fails to bind to IgM. The methods and kits are exemplified with “CSC” which is a chimeric human pIgR comprising domain 1 from a lagomorph. In one embodiment, CSC has the amino acid sequence set out in SEQ ID NO:6. In one embodiment, the methods disclosed herein use a laboratory based immune assay such as an ELISA-based assay, or a rapid point of care device as disclosed and enabled herein.

In one embodiment, a method for population surveillance or contact tracing is provided, said method comprising assessing a level of circulatory pathogen specific dIgA in a blood sample from the subject, wherein the identification of a level of pathogen specific dIgA in the blood sample indicates a time since infection/Start.

In one embodiment, a method for assessing the functional mucosal immune response to a pathogen is provided, the method comprising determining the pathogen or antigen-specific dIgA antibody level in a blood sample from a subject. As determined herein, circulating pathogen specific dIgA comprises a level of functionally neutralizing dIgA antibodies and the level of circulating dIgA provides a very useful marker for seroconversion or the production of a functional mucosal immune response at a mucosal niche. This method is enhanced by calibrating a determined dIgA level against reference longitudinal response values. As determined herein, in one embodiment, the herein disclosed assay or rapid device is able to clearly distinguish between subjects who have no, medium or high neutralizing pathogen specific dIgA levels.

In another embodiment, a method is enabled for determining/assessing the neutralizing or functional mucosal immune response at a mucosal surface against a pathogen, the method comprising measuring or determining the pathogen or antigen-specific dIgA antibody level in a blood sample from a subject.

In yet another embodiment, a method is provided for assessing the mucosal immune response to vaccination against a pathogen, the method comprising determining the vaccine-specific dIgA antibody level in a blood sample from a subject. As determined herein, the herein disclosed rapid device is able to clearly distinguish, for example, between subjects who have no, medium or high neutralizing pathogen specific dIgA levels.

In one embodiment, the method comprises comparing the antibody level to a reference response value/s to facilitate determining (i) the magnitude of the mucosal immune response to the pathogen or vaccine or (ii) the time since start, wherein the reference response value is obtained from the pre-determined level or change in the level of pathogen specific dIgA antibody over time from the blood in reference subjects.

In one embodiment, the method further comprises measuring or determining the level of pathogen specific total immunoglobulin in the sample.

In one embodiment, the dIgA binding agent is selected from: an anti-dIgA antibody, an anti-J-chain antibody, a pIgR (such as rabbit or human pIgR) or a modified pIgR that binds dIgA and substantially fails to bind IgM (such as CSC), a dIgA receptor or soluble ligand.

In one embodiment, the dIgA binding agent is a non-antibody dIgA-specific molecule.

In one embodiment, the pIgR is a chimeric human pIgR comprising a non-human domain 1, such as domain 1 derived from rabbit, wherein the chimeric human pIgR (CSC) substantially fails to bind IgM.

In one embodiment, the measured level of dIgA is assessed by visual comparison or instrument reader and/or associated software adapted to evaluate antibody levels and compare data.

In one embodiment, the method is suitable for use in a point-of-care (POC) or near POC device.

In another embodiment, the method comprises: ECLIA, IFA, ELISA-type, flow cytometry (eg, fluorescent microbeads, nanoparticles), interferometry methods, bead array, lateral flow, cartridge, microfluidic, förster resonance energy transfer, immunochromatographic based methods or formats, nucleic acid based methods (eg CRISPR) or the like.

In one embodiment, the method is performed using a laboratory based immune-assay or screen. In an illustrative embodiment the method is an ELISA-type immune assay. For example, plasma or serum obtained from a subject is combined with the dIgA binding agent CSC and incubated to form a complex. The CSC together with bound dIgA (the complex) is transferred to suitable surface upon which the pathogen or pathogen specific antigen is immobilised/bound and mixture incubated. The surface is optionally washed with buffer to remove any non-bound components. Any dIgA that specifically binds the antigen is then detected by means of the CSC. A detection reagent such as a species anti-SC antibody is incubated for a time to allow binding of the anti-SC antibody to CSC (to form a label-CSC-dIgA-antigen complex). The surface is optionally washed with buffer to remove any non-bound components. The label-CSC-dIgA-antigen complex is then detected using a labelled antibody that specifically binds to the anti-SC antibody. After washing the label is detected using a suitable procedure. For example, where the label is HRP, TMB chromogen is added to create a detectable colour change. The presence or level of antigen specific dIgA is determined from the intensity of the colour change reaction. Other approaches are illustrated in the Examples and figures.

In one embodiment, the method is performed using a POC lateral flow or microfluidic device.

In one embodiment, at least one step is performed on a lateral flow device comprising a test strip comprising at least one sample loading region, wherein the strip comprises a capture portion comprising an agent which specifically binds dIgA.

In one embodiment, the strip further comprises a further capture portion comprising an agent which specifically binds total Ig or IgG. In one embodiment, the agent is an antibody and antigen binding derivative.

In a further embodiment, the strip further comprises a further capture portion comprising a control binding agent. In one embodiment, the agent is an antibody and antigen binding derivative. In one embodiment, the control is anti-species IgY.

In one embodiment whole blood or plasma/serum is introduced to the test trip where it first meets the dIgA binding agent.

In one embodiment, pathogen specific dIgA is detected using labelled pathogen or antigen. In one embodiment the pathogen specific antigen is a neutralizing antigen, such as one comprising an RBD. In one embodiment the pathogen specific antigen is a non-neutralizing antigen.

In one embodiment, is provided a kit or POC device comprising a test strip comprising at least one sample loading region, wherein
a) the strip comprises a capture portion comprising an agent which specifically binds dIgA (and not mIgA, IgM or IgG) and directly or indirectly indicates+/−a low or medium level of pathogen specific dIgA, and
b) the strip comprises a second capture portion comprising an agent which binds dIgA (and not mIgA, IgM or IgG) and directly or indirectly indicates+/−a high level of pathogen specific dIgA.

In one embodiment is provided a kit or POC device comprising a test strip comprising at least one sample loading region, wherein
a) the strip comprises a capture portion comprising an agent which binds dIgA (and not mIgA, IgM or IgG) and directly or indirectly indicates a level of circulatory pathogen specific dIgA, and
b) the strip comprises a second capture portion comprising an agent which binds total immunoglobulin and directly or indirectly indicates a level of circulatory pathogen specific total Ig.

In one embodiment, is provided a kit or POC device comprising a test strip comprising at least one sample loading region, wherein
a) the strip comprises a capture portion comprising an agent which binds dIgA and directly or indirectly indicates a level of circulatory pathogen specific dIgA, and
b) the strip comprises a second capture portion comprising an agent which binds IgG and directly or indirectly indicates a level of circulatory pathogen specific IgG.

In one embodiment, the agent which binds dIgA, is a non-antibody agent which specifically bind dIgA and does not substantially bind mIgA, IgM or IgG. In one embodiment the binding agent is CSC.

In one embodiment, the pathogen or antigen induces a mucosal immune response or wherein the pathogenic antigen is a venom.

In one embodiment, the pathogen is an infectious organism capable of transmitting as an epidemic or pandemic, or wherein pathogen is a virus or bacteria, or fungus or parasite, or wherein the pathogen is a coronavirus such as SARS-COV-2, SARS-COV, MERS, or a potentially epidemic/pandemic coronavirus as defined herein (CoV).

In one embodiment, the antigen of a pathogen interacts with the host and promotes infectivity such as the Spike (S) protein or portion thereof, or an RBD portion of a pathogenic organism.

In one embodiment, the present application enables a method for identifying or stratifying a CoV convalescent human subject for prioritisation as a blood or plasma donor for CoV convalescent plasma therapy or purification of anti-CoV polyclonal immunoglobulin. In one embodiment, said method comprises assessing or measuring a level or presence of CoV pathogen specific dIgA in a blood sample from a subject (the donor) who has been infected with the CoV pathogen. In one embodiment, the identification of a level of CoV pathogen specific dIgA in the blood sample indicates the production by the subject of CoV pathogen specific functional (e.g. neutralizing) antibodies and the suitability of the subject for prioritisation as a blood or plasma donor for therapeutic or prophylactic convalescent plasma or immunoglobulin administration against the CoV pathogen. As determined herein, the herein disclosed rapid device is able to clearly distinguish between subjects who have no, medium or high neutralizing pathogen specific dIgA levels.

In one embodiment, the level of CoV pathogen specific dIgA is the level of CoV Spike antigen specific dIgA or a level of Spike RBD antigen specific dIgA. These antigens or epitopes therein are the target of neutralizing dIgA antibodies and therefore may be used to assess functional mucosal immune responses.

In another embodiment, the specification provides a method for population surveillance or contact tracing, said method comprising assessing a level of CoV pathogen specific dIgA in a blood sample from the subject, wherein the identification of a level of CoV pathogen specific dIgA in the blood sample indicates a time since CoV infection/Start. In this embodiment, it is not essential for the pathogen antigen to be associated with or the target of a neutralizing immune response or a host interaction such as the Spike protein or the RBD comprising region thereof.

In another embodiment, the specification provides a method for assessing the mucosal immune response to a CoV pathogen, the method comprising determining the CoV pathogen or antigen-specific dIgA antibody level in a blood sample from a subject. In one embodiment the antigen is an RBD comprising antigen.

In another embodiment, the specification provides method for determining/assessing the neutralizing or functional mucosal immune response at a mucosal surface to a CoV pathogen, the method comprising measuring or determining the CoV pathogen or CoV Spike/RBD-specific dIgA antibody level in a blood sample from a subject.

In another embodiment, the specification provides a method for assessing the functional mucosal immune response to vaccination against a CoV pathogen, the method comprising determining the vaccine-specific dIgA antibody level in a blood sample from a subject. In one embodiment, the vaccine specific dIgA antibody binds to Spike/RBD antigen.

In another embodiment, the specification provides a method for assessing the functional mucosal immune response to the CoV pathogen or to vaccination against a CoV pathogen, the method comprising determining the pathogen-specific dIgA antibody level in a blood sample from a subject and comparing the antibody level to a reference response value/s to facilitate determining one or more of (i) the magnitude of the total mucosal immune response to the CoV pathogen or vaccine in that subject (eg, the AUC) or (ii) the time since start, wherein the reference response value is obtained from the pre-determined level or change in the level of pathogen specific dIgA antibody over time from the blood in reference subjects. In the case of vaccination, the time since start is likely known so the magnitude of the total mucosal immune response to the CoV vaccine can be determined. In the case of (ii) the Spike/RBD dIgA level determined is assessed against the reliable trajectory of dIgA levels obtained from the pre-determined level or change in the level of pathogen specific dIgA antibody over time from the blood in reference subjects.

In another embodiment, the specification provides a method for assessing the functional mucosal immune response to the CoV pathogen or to vaccination against a CoV pathogen, the method comprising determining the level or presence of pathogen-specific dIgA antibody in a blood or mucosal (eg, oral and nasal swab) sample from a subject and comparing the antibody level/time since infection/vaccination to a reference response value/s to facilitate determining one or more of (i) the magnitude of the total mucosal immune response to the CoV pathogen or vaccine in that subject (eg, the AUC) or (ii) the time since start, wherein the reference response value is obtained from the pre-determined level or pre-determined time for the change in the level of pathogen specific dIgA antibody over time from blood/mucosal samples in reference subjects. In the case of vaccination, the time since start is likely known so the magnitude of the total mucosal immune response to the CoV vaccine, or vaccine challenge can be determined. In the case of (ii) the Spike/RBD dIgA level or presence determined is assessed against the reliable trajectory of dIgA levels obtained from the pre-determined level or change in the level of pathogen specific dIgA antibody over time from the blood in reference subjects.

In one embodiment, the method further comprises measuring or determining the level or presence of CoV pathogen specific total immunoglobulin or IgG in the sample. As described herein, the addition of this arm of the assay provides further information concerning the state of the subject, such as the time since infection. As the phase of amplified IgG detection in the blood occurs towards the end of the dIgA amplification phase, the presence or level of IgG and dIgA distinguishes between levels of dIgA detectable very early during infection and levels of dIgA detectable towards the end of the amplification phase, when individual levels are declining rather than increasing. This arm may be included in laboratory-based, and point of care assays and kits.

dIgA binding reagents are described herein. However, in one embodiment, the dIgA binding agent is selected or derived from: an anti-dIgA antibody, an anti-J-chain antibody, a pIgR (such as rabbit or human pIgR) or a modified pIgR that binds dIgA and substantially fails to bind IgM (such as CSC), a dIgA receptor or soluble ligand. In one embodiment, the pIgR is a chimeric human pIgR comprising a non-human domain 1 that substantially fails to bind IgM.

In one embodiment, at least part of the method is conducted in a test strip wherein the level of dIgA is assessed by visual comparison or instrument reader. In one embodiment, the associated software is adapted to evaluate anti-CoV antibody levels and compare data to generate results. Accordingly, in one particular embodiment, the method is suitable for use in a point-of-care (POC) or near POC device. Thus, the method can be rapidly deployed within different communities affected by the CoV pathogen. In one embodiment, wherein the method is performed using a lateral flow or microfluidic device.

In another embodiment, the method comprises: ECLIA, IFA, ELISA-type, flow cytometry (eg, fluorescent microbeads, nanoparticles), interferometry methods, bead array, lateral flow, cartridge, microfluidic, förster resonance energy transfer, immunochromatographic based methods or formats, nucleic acid based methods (eg crisper) or the like.

In one embodiment, the method is performed using a laboratory based immune-assay or screen. In an illustrative embodiment, the method is an ELISA-type immune assay. For example, plasma or serum obtained from a subject is combined with the dIgA binding agent, CSC and incubated to form a complex. The CSC together with bound dIgA (the CSC-dIgA complex) is transferred to suitable surface upon which the CoV pathogen or pathogen specific antigen is immobilised/bound and mixture incubated. It is also possible to directly detect CSC for example, if it is biotinylated then an avidin based detection method maybe used. Other binding pairs are known in the art.

In one embodiment, the surface is washed with buffer to remove any non-bound components. Any dIgA that specifically binds the antigen is then detected by means of the CSC. A detection reagent such as a species anti-SC antibody is incubated for a time to allow binding of the anti-SC antibody to CSC (to form a label-CSC-dIgA-CoV antigen complex). The surface is washed with buffer to remove any non-bound components. The label-CSC-dIgA-antigen complex is then detected using a labelled antibody that specifically binds to the anti-SC antibody. After washing the label is detected using a suitable procedure. For example, where the label is HRP, TMB chromogen is added to create a detectable colour change. The presence or level of antigen specific dIgA is determined from the intensity of the colour change reaction.

In one embodiment, at least one step of the method is performed on a lateral flow device comprising a test strip comprising at least one sample loading region, wherein
a) the strip comprises a capture portion comprising an agent which binds dIgA.

In one embodiment, CoV pathogen specific dIgA is detected using labelled pathogen or antigen.

In one embodiment, the application provides a kit or POC device for conducting the methods disclosed herein, comprising a test strip comprising at least one sample loading region, wherein
a) the strip comprises a capture portion comprising an agent which binds dIgA and directly or indirectly indicates+/−a low or medium level of CoV-specific dIgA, and
b) the strip comprises a second capture portion comprising an agent which binds dIgA and directly or indirectly indicates+/−a high level of CoV specific dIgA.

In one embodiment, the kit or POC device comprises a test strip comprising at least one sample loading region, wherein
a) the strip comprises a capture portion comprising an agent which binds dIgA and directly or indirectly indicates a level of circulatory CoV specific dIgA, and
b) the strip comprises a second capture portion comprising an agent which binds total immunoglobulin and directly or indirectly indicates a level of circulatory CoV-specific total Ig.

In one embodiment, the kit or POC device comprising a test strip comprising at least one sample loading region, wherein
a) the strip comprises a capture portion comprising an agent which binds dIgA and directly or indirectly indicates a level of circulatory CoV specific dIgA, and
b) the strip comprises a second capture portion comprising an agent which binds IgG and directly or indirectly indicates a level of circulatory CoV specific IgG.

In one embodiment, the method uses a kit comprising dIgA (CSC) that specifically detects the presence or level of circulating antigen-specific dIgA in a subject to determine:
(i) time since Start or infection of a subject
(ii) the presence of a functional (ie, neutralizing) mucosal immune response to the pathogen in a subject
(iii) the presence of no, low, medium of high levels of neutralizing dIgA antibodies
(iv) the magnitude of the mucosal immune response in a subject to a pathogen or vaccine
(v) the stratification of convalescent subjects for prioritisation as blood/plasma donors to prepare convalescent serum or dIgA preparations for use in therapy to provide neutralizing antibodies and a functional mucosal response to reduce pathogen levels/infection.

In one embodiment, there is provided a method of treating or preventing an infection with a pathogenic organism or the pathogenic condition associated therewith, the method comprising administering convalescent plasma or other blood product selected for high or elevated levels of pathogen specific dIgA.

In one embodiment, there is provided a method of treating or preventing an infection with a pathogenic organism or the pathogenic condition associated therewith, the method comprising administering convalescent plasma or other blood product selected based on the levels of pathogen specific dIgA. In one embodiment the pathogen specific dIgA is specific to a neutralizing antigen of the pathogen.

In one embodiment the method comprises (i) measuring circulating antigen-specific dIgA levels in a blood sample from a convalescent subject, as defined herein and (ii) preparing convalescent plasma from the subject and (iii) administering convalescent plasma to a subject in need thereof.

In one embodiment, the present application enables a method of treating or preventing an infection by a pathogen, comprising administering convalescent plasma to a subject in need thereof, wherein the convalescent plasma donor is selected and based on the presence of circulating pathogen specific dIgA within 1 to 3 months of infection.

In one embodiment the pathogen specific dIgA is specific to a neutralizing antigen of the pathogen.

In one embodiment, convalescent plasma so identified is processed to enrich for pathogen specific dIgA. In one embodiment, pathogen specific dIgA is purified from the plasma using a dIgA-specific binding affinity purification step and stored in a suitable format. In one embodiment the dIgA is formulated as a physiological or pharmaceutically acceptable composition.

In one embodiment, the present disclosure enables a method of assessing in a biological sample from a subject the functional mucosal immune response eg. the antigen specific neutralizing capacity of a subject, the method comprising measuring or determining the presence or level of SIgA in a biological sample such as but not limited to a mucosal sample using there herein described assays and kits. For example the binding between SIgA and antigen is detected using anti-SC. In this assay, the presence or level of antigen specific SIgA is directly correlated with the presence or level of a functional mucosal immune response.

In a further aspect the present application provides an in vitro serology assay or kit for detecting or excluding the presence of a complex or first complex comprising (i) dIgA from within a blood sample from a subject (e.g, whole blood, plasma, serum), and (ii) a pre-prepared respiratory virus or an antigen of the respiratory virus, and/or (iii) a pre-prepared SC (secretory component) or pIgR-based protein that specifically forms a complex with dIgA and not IgA or IgM or IgG from within the blood sample, wherein the blood sample is contacted with (ii) and (iii) in the kit or assay and the complex detected visually or by instrument via a label or other detectable moiety to (ii) and/or (iii).

In view of the observed predictable decline in dIgA levels shortly after the acute phase, in one embodiment detection of a complex comprising dIgA indicates the maximum time since infection.

In view of the observed predictable decline in dIgA levels during the tail end of the acute phase and/or shortly after the acute-phase, in one embodiment detection of a complex comprising dIgA indicates the maximum time since infection.

In one embodiment, the level of dIgA or a complex comprising dIgA indicates the time or maximum time since infection.

In one embodiment repeat dIgA testing may be conducted within a few days to establish that a low positive is a very early acute infection. Repeat testing may be conducted within a short time and provide useful results. This is in contrast to IgM or IgG testing where levels do not typically rise so quickly.

In one example, a patient with symptoms consistent with SARS-COV-2 infection or a known or probable contact with an infectious source, a low- or moderate-level of dIgA might be present in the first one or two days of the increasing dIgA response. In this example a positive dIgA of any level indicates a recent infection with SARS-COV-2. Therefore, a positive dIgA (of any level) in a patient where there is clinical suspicion of acute SARS-CoV-2 is diagnostic. In one embodiment, a positive dIgA test (of any level) in any patient where there is suspicion of acute or recent SARS-COV-2 infection would be indicative of recent infection. This might be demonstrated by the contemporaneous request or requirement for testing for viral RNA(PCR) or viral antigen test.

As the skilled person will appreciate and as discussed herein, the ability to determine time since infection is highly useful in, for example, contact tracing. While the present data provide very reliable trends for CoV specific infections, other pathogens are anticipated to display altered specific time lines within the general trends determined herein. Individual patients with different kinetics of response would be expected and do not disprove the general model.

For the avoidance of doubt, in one broad embodiment the method or kit only detects pIgR bound dIgA and does not detect another antibody or total immunoglobulin. In one embodiment, the method or kit does not additionally detect an antigen. In some embodiment as described further herein, the methods and kits detect further immunoglobulins, total immunoglobulin and/or antigens.

In one embodiment, the assay or kit is sensitive for detecting the respiratory viral infection via detection of the first complex during an early post-acute phase of recent infection when molecular testing for virus or antigen displays reduced sensitivity for detecting recent infection.

In one embodiment, the respiratory virus is a coronavirus (CoV), influenza virus or respiratory syncytial virus.

In one embodiment, the antigen is a CoV Spike antigen.

In one non-limiting embodiment, the coronavirus antigen is Spike protein, a sub-domain or antigenic/antibody binding portion of the Spike protein e.g., comprising all or part of the S1 subunit or the S2 subunit. The S1 subunit comprises, inter alia, the N-terminal domain and the RBD which are important for antibody binding. In one embodiment, the antigen comprises at least the S1 subunit or at least the N-terminal domain or at least the N-terminal domain and the RBD of Spike protein. All such alternatives are referred to herein as Spike antigens.

Combined dIgA/antigen testing is contemplated as described herein.

In a still further embodiment, the present application provides the assay or kit for detecting or excluding (A) the presence of a complex or first complex comprising (i) dIgA from within a subjects blood sample (e.g, whole blood, plasma, serum), and (ii) a pre-prepared respiratory virus or an antigen of the respiratory virus, and/or (iii) a pre-prepared SC (secretory component) or pIgR-based protein that specifically forms a complex with dIgA and not IgA or IgM or IgG from within the blood sample, wherein the blood sample is contacted with (ii) and (iii) in the kit or assay and the complex detected visually or by instrument via a label or other detectable moiety to (ii) and/or (iii) and (B) the presence of a second complex comprising (iv) a respiratory virus antigen from within a blood sample of a subject (e.g, whole blood, plasma, serum), and (v) one or more pre-prepared binding agent, antibody or antibody derivative that specifically recognise/s the antigen in (iv), wherein a blood sample is contacted with (v) and the second complex detected visually or by instrument via a label or other detectable moiety.

In one embodiment of this dIgA/Antigen test, antigen (ii) in the first complex is derived from a different protein of the respiratory virus to antigen (iv) detected in the second complex.

In one embodiment, the first complex comprises a Spike antigen and the second complex comprises a nucleoprotein antigen.

In one embodiment, in the combined test described above or in the examples, the assay is completed using a single blood sample to detect or exclude the presence of the first and second complex in a single assay, single test strip or plural test strips, or wherein the assay is completed using separate blood samples to detect each complex.

Methods are also provided that employ dIgA testing.

In one embodiment, the present application provides an in vitro method for screening a blood sample or a mucosal surface sample, or both, and detecting a recently active viral respiratory infection at a time post-acute infection when molecular and antigen tests display reduced sensitivity for active infection, the method comprising using the assay or kit described herein (test dIgA testing) to detect or exclude the presence of a first complex as defined herein.

In one embodiment, the level of pathogen specific dIgA during an early post-acute stage/phase of a recent infection with the pathogen reliably indicates whether or not the subject has had a recent infection with the pathogen. The method wherein the rising level of pathogen specific dIgA during an early post-acute phase of a recent infection with the pathogen at a time when pathogen levels are declining or not detectable, reliably indicates whether or not the subject has had a recent infection

In one embodiment of the method, results from practising the method are combined with molecular or antigen testing results and the combined results are stored on a computer and used to more accurately determine recency of infection compared to using either test alone.

Methods are contemplated using circulating dIgA testing or circulating dIgA testing combined with blood/plasma antigen testing, or using circulating dIgA testing combined with antigen testing and/or molecular testing.

In one embodiment the present application provides an in vitro method for reducing forward transmission of a contagious agent/respiratory viral infection by directing subjects identified as having recent or active infections to self-isolate, or vaccinate or medicate, or other temporary or long term behavioural change, the method comprising using the assay or kit as described herein above to detect or exclude the presence of a complex as defined herein above.

Methods may further include testing for a second or multiple further antibody classes.

In one embodiment, the circulating dIgA-based assay or kit further comprises reagents for detecting one or more of antigen-specific IgG, IgA, IgM, total Ig from within the blood sample.

In another aspect, the present application provides a dIgA antibody recognising SARS-COV-2 having a sequence as set out in one or more of SEQ ID NO: 21 to 24. A dIgA antibody recognising SARS-COV-2 having a sequence as set out in one or more of SEQ ID NO: 21 to 24 or a sequence having 98% sequence identity thereto, or comprising 1-3 conservative substitutions.

### BRIEF DESCRIPTION OF THE FIGURES

FIG. 1A) shows monomeric and dimeric IgA forms and their % of total IgA in normal human blood. Monomeric IgA shows levels of approximately 1.9 mg/mL serum, dimeric IgA approximately 0.2 mg/ml, and Secretory IgA has a very low level in human serum being found primarily in the mucosa. Dimeric IgA (dIgA) is the major form of IgA produced by IgA-secreting plasma cells, found mainly in the lamina propria of mucosal sites. The inventors have determined herein that these cells are found in the circulation for a period after acute infection, hence the dIgA is present in blood not only as SIgA in mucosa. This important discovery has broad implications not limited to CoV in the area of epidemiology, immunology, monitoring vaccine efficacy, therapeutics and diagnostics. dIgA has a relatively short half-life in plasma due to rapid secretion as Secretory IgA. Monomeric IgA is largely produced in the bone marrow and circulating B-cells, and is unrelated to mucosal immune responses. B) The applicants have recently developed (International Patent Publication no. WO 2014/071456 in the name of Macfarlane Burnet Institute for Medical Research and Public Health) a derivative of human pIgR (referred to as chimeric secretory component or CSC) and shown that it is efficient for discriminating dIgA from the 10-fold more abundant monomeric IgA in blood samples. Also, CSC, which comprises domain 1 from another species (e.g. rabbit whose pIgR does not bind IgM, or rat) does not bind IgM or only negligibly, which therefore strengthens specificity. A diagrammatic representation of specific dIgA detection as described herein. In this illustrative example, CSC is bound to a solid support and binds to dIgA forming a receptor-antibody complex. The complex can be visually detected with anti-human IgA colloidal gold, or antigen-colloidal gold. This detects the dIgA from blood/plasma before it is secreted as SIgA at the mucosa.

FIG. 2 shows a diagrammatic representation of CSC used in the detection of dIgA as described herein. R/HpIgR, or CSC is a chimeric pIgR comprising domain 1 of rabbit pIgR and domains 2 to 5 of human pIgR. The panel on the left shows HpIgR which binds IgM and dIgA. The middle panel shows R/HpIgR with domain 1 indicated in a different shade. The Right panel shows Coomassie staining of an SDS page gel of recombinant expressed CSC before purification (SN) or after purification on nickel affinity column (Pure).

FIG. 3 shows a diagrammatic representation of one example of the ELISA protocol as described herein. In this example, the test kit is coated with recombinant SARS-COV-2 full-length Spike protein. In the first complexing step, the Chimeric Secretory Components (CSCs) bind to dimeric IgA (dIgA) of the diluted patient samples. After addition of this complexed sample to the test kit, specific dIgA-CSC complex (also IgG and IgM) antibodies in positive samples, will bind to the antigen. To detect the bound dimeric IgA-CSC complex, a mouse monoclonal detecting antibody will bind to the CSC of the CSC/dIgA complex. A labelled anti-mouse IgG (enzyme conjugate) is then added to detect the bound antibodies, catalysing a colour reaction. Note that the detecting antibody may alternatively be added to the CSC prior to addition to the patient sample to form the dIgA-CSC-detecting antibody complex in one step, because a preferred detecting antibody will not interfere with the formation of the dIgA-CSC complex.

FIG. 4 shows the results of a dIgA ELISA as described in FIG. 3 performed with the full-length Spike protein antigen. A) Compares the dIgA levels in 30 controls (0/30 Hep C and healthy control samples tested positive) and 60 COVID-19 positive samples (COVID-19 was detected in 44/60 (73%) of positive samples). Mean±3 SD of controls, the cut-off value is OD 0.28. B) Shows ELISA dIgA S full length vs days post PCR diagnosis. A significant but modest correlation of dIgA ELISA reactivity and time after infection/diagnosis is shown in this preliminary data.

FIG. 5 shows the results of a dIgA ELISA as described in FIG. 3 but with Spike RBD antigen instead of full-length spike antigen, and days post PCR diagnosis. The graph shows that dIgA is detectable as early as 2 days after PCR diagnosis, and is consistently elevated in most patients between that time and 60 days after PCR diagnosis, but then becomes negative in most patients by more than 80 days after PCR diagnosis. A significant correlation of dIgA ELISA reactivity and time after diagnosis is shown.

FIG. 6A) is a diagrammatic representation of the dIgA lateral flow assay (LFA) Point-of-care protocol and illustrative reagents. dIgA from the patient sample is captured with the CSC reagent (0.4 μl/cm of 1 mg/ml recombinant CSC with His Tag expressed in Freestyle 293F cells, and purified by nickel affinity and size exclusion chromatography to >95% purity (dimer)). Patient IgG is captured with anti-human IgG Fc. Goat anti-chicken IgY serves as a procedural control. Antigen-specific complex dIgA and/or IgG is detected with Streptavidin-40 nm gold (Abcam/Expedeon) and Burnet RBD-biotin (comprising the ancestral strain receptor binding domain with C-terminal poly-histidine tag and avitag allowing site-specific biotinylation) expressed in Freestyle 293F cells stably transfected with BirA ligase and purified by nickel affinity and size exclusion chromatography to >95% purity. The procedural control is detected using 40 nm gold conjugated with Chicken IgY (BBI, UK). Antigen-specific complex dIgA and IgG can also be detected using full-length N protein (Genscript) and 40 nm gold conjugated with anti-N monoclonal antibody (EastCoast Bio). B) Shows representative lateral flow strips with 8 different negative samples or 2 different positive samples (P1 and P2) detected with N protein gold conjugate (left panel), showing detection of the procedural control line in all samples, strong detection of IgG and strong (P1) or weak (P2) reactivity of dIgA lines in the positive samples, but no reactivity for IgG or dIgA for the negative samples. Right panel shows reactivity of P1 sample with 2 different batches of RBD antigen gold, again showing strong reactivity of both IgG and dIgA.

FIG. 7 shows the results of a dIgA lateral flow assay using the RBD-biotin antigen for early and late convalescent plasma samples of patients confirmed to be infected with SARS-COV-2 obtained from a biobank. Once applied to the strip, the plasma sample will flow laterally across the strip contacting the dIgA binding reagent (CSC), followed by the IgG binding reagent, followed by the control binding reagent. In this assay 0/30 Hep C and healthy control samples tested positive and 22/58 (38%) COVID-19 samples tested positive, but 9/12 samples at less than 55 days after PCR diagnosis tested positive consistent with acute-phase reactivity of dIgA. Visual cutoff (=400 Axxin units) is represented by the dashed line.

FIG. 8 shows the results of an alternative format of dIgA lateral flow assay with dIgA detected the same way as FIG. 7, but total antibody detected with double-antigen sandwich using RBD to capture total RBD-specific antibody, and RBD-biotin to detect the bound RBD-specific antibody. A) shows the reagents used that are mixed with the 5 μl plasma sample (Chicken IgY gold and RBD-biotin/streptavidin gold), and the reagents on the test strip (CSC, RBD and anti-Chicken IgY). B) Shows a representation of the reactions that occur when RBD-specific dIgA and total antibody (such as IgG) are present in the sample. Once applied to the strip the sample contacts the dIgA binding reagent (CSC), followed by the immobilised RBD, followed by the detection reagent (Burnet RBD-biotin, as for the dIgA test line). The control binding reagent is anti-Chicken IgY/Chicken IgY gold as before. C) Examples of test strips from known positive and negative COVID-19 cases.

FIG. 9A) shows the results of a dIgA lateral flow assay with both dIgA and total RBD-specific antibody detected with RBD-biotin. B) Shows the results of a dIgA lateral flow assay detected with RBD-biotin correlated with days post PCR diagnosis. The results show a strong correlation (R²=0.4) of dIgA with days after COVID-19 diagnosis. 16/22 (73%) tested positive for COVID-19≤55 days and there was a trend towards testing negative after ≈80 days. Visual cutoff (=400 Axxin units).

FIG. 10 shows the correlation of dIgA lateral flow assay results and dIgA ELISA assay results when antibody is detected with SARS COV-2 RBD-biotin (lateral flow) or SARS COV-2 RBD (ELISA). For the samples that were shown in FIG. 9, very close correlation is observed for dIgA ELISA reactivity and dIgA lateral flow activity for samples with an ELISA OD of less than 3, but for samples with an ELISA OD of more than 3 there is reduced relative level of dIgA lateral flow activity (while still strongly positive), consistent with a prozone effect in the lateral flow assay in the presence of excess RBD-specific antibody.

FIG. 11 A) shows the correlation of dIgA lateral flow assay detected with RBD-biotin versus days post PCR diagnosis after correction for the prozone effect. For the samples shown in FIG. 9 and FIG. 10, samples that were showing evidence of a prozone effect (dIgA ELISA OD>3) were assigned a nominal value of 15,000 Axxin units equivalent to the maximum reading of samples without prozone effect, The results show an increasingly strong correlation (R²=0.51 vs R²=0.4 before prozone correction) between the level of dIgA and days after COVID-19 PCR diagnosis. 16/22 (73%) tested positive for COVID-19≤55 days and there was a trend towards testing negative after ≈75 days. Visual cutoff (=400 Axxin units). B) shows the dIgA ELISA (bars) and lateral flow assay (line) results for one patient with samples collected at 57, 61, 64 and 67 days after PCR diagnosis, showing the very rapid decline of dIgA levels detected by either method, consistent with the loss of RBD-specific dIgA-producing B cells from the peripheral circulation by this time, and subsequent rapid export of dIgA to the mucosa as SIgA, and thus clearance of the preformed dIgA from plasma.

FIG. 12 illustrates the strong correlation of the level of dIgA ELISA reactivity versus time after symptom onset in a separate cohort of patients with PCR-confirmed SARS-CoV-2 infection. A similar strong correlation is seen between the RBD-specific dIgA level and time after symptom onset (R²=0.56), with a peak at around 25 days after onset. The earliest detection of dIgA in this cohort was at 7 days after symptom onset, with 100% sensitivity (21/21) from day 12 to day 29 after symptom onset (95% CI: 86-100%) again consistent with the acute phase reactivity of antigen-specific dIgA.

FIG. 13 shows the data values for FIG. 12, and comparison with the ELISA reactivity with the EuroImmun SARS-COV-2 IgG ELISA. As shown in FIG. 12, the dIgA ELISA detects reactivity as early as 7 days after onset and is positive in all patients (21/21) from 12-29 days post symptom onset, whereas the EuroImmun IgG ELISA is first positive at 10 days after symptom onset, and is negative in 2/21 patients from 12-29 days post symptom onset.

FIG. 14A) shows dIgA lateral flow assay reactivity (Axxin units) over time after PCR diagnosis of SAR-COV-2 with strong correlation (R²=0.39) and a trend towards patients becoming negative by around 75 days. B) Shows the same data for correlation of dIgA versus the lack of correlation seen for IgM in a lateral flow assay over time after PCR diagnosis of SARS-COV-2 (R²=0.01 for IgM detection v 0.39 for dIgA detection). In this example, RBD-specific IgM is detected by using anti-IgM as the capture reagent instead of CSC, but similar results are seen if using HSC (which captures both IgM and dIgA) instead of CSC (which captures only dIgA). Consistent with emerging literature, IgM reactivity does not show a strong association with acute-phase SARS-COV-2 infection.

FIG. 15 shows the measurement of neutralizing antibodies in longitudinal plasma samples from a convalescent COVID-19 subject. It shows that neutralizing antibodies can be detected in convalescent plasma obtained from a person with confirmed SARS-COV-2 infection, but not in a sample of plasma obtained from a different individual prior to December 2019 who did not have SARS-COV-2 infection (“negative” pre-COVID-19). The strength of neutralization gradually wanes over time in this individual, with the highest titre of neutralizing antibody detected in sample 1, earliest time point after infection, and the lowest titre detected in sample 5, the longest time point after confirmed SARS-COV-2 infection.

FIG. 16 shows the neutralizing activity (reciprocal titre of antibody required for 50% reduction in viral entry=ID50) of Biobank plasma samples from COVID-19 subjects. The data show the ID50 titres of 58 convalescent plasma samples obtained from a Biobank of COVID-19 subjects.

FIG. 17 shows the half-life of neutralizing antibodies in one subject over time (upper panel) and the longitudinal analysis of neutralizing antibody titres in an additional five patients in the days post PCR diagnosis of SARS-COV-2. This shows that neutralizing antibody responses change rapidly over time in many patients.

FIG. 18 shows the stratification of patients based on levels of neutralizing antibodies (NAbs) and A) days post diagnosis (no difference), B) patient age (no difference) C) presence of total Ig to RBD (strong difference) and D) presence of IgG to S1, measured with EuroImmun S1 ELISA (strong difference).

FIG. 19 shows the stratification of patients based on levels of neutralizing antibodies and IgA levels measured by Euroimmun IgA to S1 using ELISA, dimeric IgA to RBD using ELISA and dimeric IgA to RBD-biotin in the rapid (lateral flow) test. It shows a comparison of NAb titres with levels of IgA A) and dimeric IgA B) and C). The neutralizing antibody (NAb) levels of 58 subjects from a biobank of COVID-19 convalescent plasma were measured and then stratified into three groups. The “no NAbs” group had no detectable ability to prevent 50% inhibition of virus entry at the lowest dilution of plasma tested (1/20). The second group had ID50 titres of between 20 and less than 300 (moderate titre). The third group had high NAb titres of >300 (high titre). Both the Euroimmun IgA test and the dIgA ELISA were able to discriminate between patients with no NAbs versus those with moderate or high titre, but could not discriminate between those with moderate and those with high titre in this analysis. The dIgA rapid (lateral flow) test was able to discriminate all three groups of patients from each other, including between those with moderate and those with high titre antibodies.

FIG. 20 shows A) that CSC beads were able to preferentially bind dIgA in an immunoprecipitation pull-down experiment, with most of the dIgA found in the “bound” rather than the supernatant fluid (“SNF”), with a much lower binding efficiency towards IgG and IgM isotypes (most of the IgG and IgM in the “SNF”) under these experimental conditions. Note that dIgA and pentameric IgM appear as their monomeric forms in the reducing agent SDS-PAGE gels. B) Shows the quantitation of the SDS-PAGE and demonstrates that approximately 80-90% of the dIgA could be bound by CSC-beads whilst only approximately 20% of IgM and IgG was associated with CSC-beads. C) and D) show that CSC-beads depleted the neutralization activity in the plasma after either 2 h or overnight incubation with convalescent plasma and reduced the ID50 titre approximately 70-80% compared to the BSA control depletion. Protein G Sepharose beads which do not bind IgA, instead binding IgG isotypes strongly, depleted approximately 25-50% of the neutralization activity in the plasma. This indicates that the majority of the neutralization activity in the plasma samples is of the dIgA isotype.

FIG. 21 shows the correlation of dIgA (RBD) by ELISA and neutralizing antibody titres for longitudinal samples (n=6 patients, 8 or 9 samples per patient). Strong correlation of dIgA levels versus neutralizing antibody titres was observed (R²=0.6793).

FIG. 22 shows strong correlation of dIgA levels versus neutralizing antibody titres was observed with a dIgA ELISA detecting RBD. Using a dIgA cutoff of ELISA OD 1.5 (sample/cutoff ratio value of 2.6) (samples highlighted in green) gives 94% sensitivity (17/18), and 97% specificity (31/32) for samples with either high (>300), or extremely high levels of NAb (>900 titre).

FIG. 23A) shows addition of plasma and B) shows addition of running buffer to the lateral flow immunochromatographic assay (rapid test) for the SARS-COV-2 total Ig and dIgA test in FIGS. 9, 10, 11, 14 and 19. C) Shows the visual interpretation of the test results for the assay described in FIGS. 9, 10, 11, 14 and 19. a) Total Ig positive, dIgA positive b) Total Ig positive, dIgA negative, c) Total Ig negative, dIgA positive d) Total Ig negative, dIgA negative (e-h). Invalid test results because of the absence of the procedural control (C) line.

FIG. 24 shows the advantage of sampling whole blood for dIgA as a marker of mucosal immune response in the early convalescent phase or early after immunization, rather than sampling mucosal surfaces such as saliva for SIgA. A) Early after infection or immunisation, B-cells producing pathogen-specific dIgA are mobilized and expanded in the peripheral circulation, excreting pathogen-specific dIgA into the blood where it can readily be sampled and detected using the methods described herein. The dIgA produced in the circulation is exported as SIgA when it comes into contact with mucosal epithelia, and will become undetectable once all the relevant B-cells migrate to the mucosal lamina propria. The amount of pathogen-specific dIgA in the peripheral circulation at this time, including the AUC, will reflect the number and functional capacity of such B-cells that are destined to migrate to the mucosal lamina propria. Exported SIgA can be detected in saliva and other mucosal samples at this time, but is subject to the inconsistent sampling errors and dilution in these samples. B) Later after infection or immunization, the dIgA-producing B cells have homed to the mucosal lamina propria, where they continue to produce pathogen-specific dIgA but because of their close location to the epithelia, the dIgA is efficiently exported as SIgA and does not enter the peripheral circulation in detectable amounts. B-cells can be detected in the tissue, and exported SIgA can be detected in saliva and other mucosal samples at this time, but both of these measures are subject to inconsistent sampling errors, sample dilution and inconvenience of tissue sampling.

FIG. 25 diagrammatically shows the assay principle of the Anti-SARS-COV-2 ELISA (dIgA). Here, mouse anti-SC is used to detect SC; HRP labelled anti-mouse IgG is used to detect the mouse anti-SC and TMB chromogen colour change is used to assess binding of antigen/pathogen to dIgA. Other suitable detection arrangements are known in the art, some of which are described herein.

FIG. 26 shows the assessment of antibody levels in three COVID-19 affected individuals. Total antibody levels and dimeric IgA were measured using a lateral flow test and neutralizing antibodies towards SARS-COV-2 (ancestral Hu-1 spike) was performed using retroviral pseudotyped virus. The results show that neutralizing antibody levels parallel dIgA levels closely and to a lesser extent total immunoglobulins towards RBD antigen.

FIG. 27 illustrates construction of chimeric IgA using known variable domain sequences of any antibody. Vectors for the expression of the IgA heavy chain were constructed to allow the insertion of the variable domain of any antibody. The heavy chain vector is co-expressed with the kappa light chain vector for the expression of monomeric IgA. The inclusion of the J chain in the heavy and light chain vector mixes allows for the expression of dimeric IgA. Both IgA1 and IgA2 versions were constructed. A test case using human monoclonal antibody CB6 (Shi et al Nature 2020) was constructed. CB6 is a potent neutralizing MAb isolated from a convalescent patient with a reported IC50 of 40 ng/ml and blocks SARS-COV-2 entry via direct blockade of the ACE2 receptor. Macaques infused with 50 mg/kg CB6 were reported to have a >3.5 Log reduction in viral loads in macaques.

FIG. 28 shows expression of different CB6 IgA forms in 293 Expi cells. 293 Expi cells were transfected with CB6IgA1 heavy and CB6 kappa light chain (IgA1), CB6IgA2 heavy and CB6 kappa light chain (IgA2), CB6IgA1 heavy and CB6 kappa light chain and J chain (IgA1+J) or CB6IgA2 heavy and CB6 kappa light chain and J chain (IgA2+J). Lanes marked supernatant fluid (SNF) show direct analysis of 10 ul of transfected cell culture supernatant on reducing SDS-PAGE gel and stained with Coomassie blue. The position of the heavy and light chains is indicated on the right. Confirmation that dimeric IgA was expressed from transfected cells was performed by incubation of 500 ul SNF with 50 ul CSC coupled to Sepharose (CSC-beads). After a 2 hour incubation at room temperature, CSC-beads were washed 5 times with PBS and then reducing sample buffer added and run on SDS-PAGE. The results show that when the J chain is present in the transfection mix, both the heavy and light chain are captured by the CSC-beads and detected with Coomassie blue staining.

FIG. 29 shows the assessment of monoclonal CB6IgA species in COVID-19 dIgA lateral flow assay. The format of the lateral flow assay is shown on the left hand side. 10 ul of SNF was applied to COVID-19 LFA cartridges. Results show that supernatant fluid (SNF) obtained from CB6IgA1 heavy and CB6 kappa light chain (IgA1) and CB6IgA2 heavy and CB6 kappa light chain (IgA2) transfections binds to RBD and is detected in the Total immunoglobulin line. No dimeric IgA is present. SNF obtained from transfections with CB6IgA1 heavy and CB6 kappa light chain and J chain (IgA1+J) or CB6IgA2 heavy and CB6 kappa light chain and J chain (IgA2+J) bind strongly to the dIgA line detecting RBD specific dIgA antibody, and weakly to total RBD specific immunoglobulin, consistent with these transfections producing a purely or predominantly dimeric CB6IgA species.

FIG. 30 shows neutralization of SARS-COV-2 pseudotyped viruses with CB6IgA species. SNF obtained from 293 Expi cells transfected with CB6IgA1 heavy and CB6 kappa light chain (CB6 IgA1), CB6IgA2 heavy and CB6 kappa light chain (CB6 IgA2), CB6IgA1 heavy and CB6 kappa light chain and J chain (CB6 dIgA1) or CB6IgA2 heavy and CB6 kappa light chain and J chain (CB6 dIgA2) were used in a neutralization assay with SARS-COV-2 pseudotyped viruses. Serial dilutions of SNF were mixed with an equal volume of pseudotyped viruses and used to infected 293-ACE2 cells. Luciferase activity was measured after 3 days. The results show that supernatant fluid containing monoclonal CB6IgA1, CB6IgA2, CB6dIgA1 and CB6dIgA2 have neutralizing activity. For comparison, purified CBG IgG1 neutralization curve is shown, starting at 2 ug/ml.

FIG. 31 shows size exclusion chromatography of CB6IgA forms expressed in 293 Expi cells. 293 Expi cells were transfected with CB6IgA1 heavy and kappa light chain (IgA1), CB6IgA2 heavy and kappa light chain (IgA2), CB6 IgA1 heavy and kappa light chain and J chain (IgA1+J) or CB6IgA2 heavy and kappa light chain and J chain (IgA2+J). To 30 ml supernatant fluid, ammonium sulfate was added to a final concentration of 45%. After end over end rotation at 4° C. overnight, the precipitate was pelleted at 20,000 g and resuspended in PBS. The redissolved proteins were buffer exchanged and then loaded onto a Superose 6 increase 10/30GL column. Profiles show that the presence of the J chain causes an increase in size, and disappearance of the ˜160 kDa species, confirming that in transfections of (A) IgA1 heavy and CB6 kappa light chain and J chain (IgA1+J) or (B) IgA2 heavy and CB6 kappa light chain and J chain (IgA2+J) dimeric IgA is formed. The peak fractions were collected, pooled and concentrated. 5 ul of concentrated protein was analysed on 12% SDS-PAGE and stained with Coomassie blue (C). SDS-PAGE reveals the presence of heavy and light chain for CB6 IgA1, CB6 dIgA1, CB6 IgA2 and CB6 dIgA2.

FIG. 32 shows neutralization of SARS-COV-2 pseudotyped viruses with purified CB6 IgA species. Purified CB6 IgA species after gel filtration were used in a neutralization assays with SARS-COV-2 pseudotyped viruses. Serial dilutions of purified CB6 IgA1, CB6 dIgA1, CB6 IgA2 and CB6 dIgA2 were mixed with an equal volume of pseudotyped viruses and used to infected 293-ACE2 cells. Luciferase activity was measured after 3 days. Plots of percentage entry against serial dilution of antibody are shown (A) and the resultant IC50 for each species (B). The results show that dimeric CB6 IgA1 and dimeric CB6 IgA2 have approximately 10-fold higher activity than monomeric CB6 IgA counterparts. For comparison, purified CB6 IgG1 neutralization curve is shown, starting at 2 ug/ml.

FIG. 33 shows development of a rapid lateral flow point of care assay for the detection of dIgA antibodies in plasma to SARS-COV-2. A. Schematic of proteins striped onto nitrocellulose for use in LFA. B. Schematic of LFA test showing complexes formed at each stripe visible using 40 nM gold conjugated detection reagents. C. Photograph of LFA test showing detection of dIgA (CB6 J+A1 and CB6 J+A2). D. Serial dilution of monoclonal IgA and IgG species showing dose dependent detection of immunoglobulins in the Total Ig stripe, and highly specific and sensitive detection of detection of dIgA1 and dIgA2 on the CSC stripe. E. Specificity of dIgA LFA. 171 pre-COVID plasmas and 22 PCR confirmed SARS-COV-2 negative samples but with known history of non SARS-COV-2 coronavirus infection were analysed using the LFA and measured as above. F. Specificity of total Ig LFA. 171 pre-COVID plasmas and 22 PCR confirmed SARS-COV-2 negative samples but with known history of non SARS-COV-2 coronavirus infection were analysed using the LFA and measured as above. G. Analysis of a 112 cross-sectional convalescent symptomatic COVID-19 subjects for the presence of dIgA. H. Analysis of dIgA over time in a cross sectional cohort. Linear regression analysis was performed in Prism v 9 and plotted with 95% confidence intervals. I. Schematic of proteins striped onto nitrocellulose for use in LFA adapted for whole blood. A anti glycophorin A sample pad was placed over the sample pad containing RBD-biotin, streptavidin-AU, and chicken IgY-AU. J. Examination of performance of dIgA assay for whole blood and plasma. Reconstituted whole blood or plasma was spiked with IgG and various amounts of dIgA and applied to the sample port with buffer. K. Examination of performance of dIgA assay in clinical samples. Early convalescent SARS-COV-2 (Pos) or pre-COVID-19 (Neg) plasma was tested directly (plasma) or after reconstitution as whole blood (WB).

FIG. 34 shows detection of SARS-COV-2 specific immunoglobulins in 47 subjects collected intensively post symptom onset. A. Number of dIgA positive samples and mean LFA read over time. Data for each time point were averaged and plotted against days post-symptom onset for days up to day 25. Day 0 includes samples from between days-2-0. A beta decay curve fitted to the data using Prism v9. Arrow indicates when 100% of samples tested positive for each assay type. B. Number of IgG positive samples using the EDI IgG test and mean OD over time. A beta decay curve fitted to the data using Prism v9. Arrow indicates when 100% of samples tested positive for each assay type. C. Correlation of IgG and dIgA results. A non-parametric Pearson correlation analysis was performed in prism v9.02. D. Number of IgM positive samples using the EDI IgM test and mean OD over time. Arrow indicates when 100% of samples tested positive for each assay type. E. Correlation of IgM and dIgA results. A non-parametric Pearson correlation analysis was performed in prism v9.02.

FIG. 35 shows A. Schematic of vectors encoding chimerized DNA sequences for the expression of CB6-IgA1, CB6-IgA2, CB6-dIgA1 and CB6-dIgA2 immunoglobulins. B. Transfection conditions for expression in 293 Expi cells at 30 ml scale. C. Schematic of IgA species used to validate lateral flow test.

FIG. 36 shows purification of IgA and dIgA species. Ammonium sulfate precipitated CB6-dIgA1 and monomeric CB6-IgA1 (A) and CB6-dIgA2 and monomeric CB6-IgA2 (B) were buffer exchanged by dialysis into PBS and proteins purified by size exclusion chromatography on a Superose 6 10/30GL column. Peaks corresponding to the predicted size of dIgA1/2 and monomeric IgA1/2 were collected, concentrated and examined in SDS-PAGE (C). Protein L purified IgA species were affinity purified using Protein L and examined by 8-15% SDS/PAGE either non-reduced or reduced.

FIG. 37 shows quantification of dIgA and total Ig levels in whole blood or plasma using the dimeric IgA LFA. Samples showin in FIG. 33J were quantitated in an Axxin reader.

FIG. 38 shows analysis of dIgA in laterial flow assay (A), IgM using EDI ELISA (B), and IgG using EDI ELISA (C). Plasma samples obtained at various times since symptom onset from 45 subjects were examined for the presence of each antibody type.

FIG. 39 illustrates the advantage of combining dIgA with RT-PCR or antigen detection

FIG. 40 from Kucirka et al, Ann Intern Med. doi: 10.7326/M20-1495, shows the proportion NEGATIVE by RT-PCR at different times post onset in confirmed infections. The antigen positivity is inferred as being 80% of that for RT-PCR at any given time.

FIG. 41 graphically illustrates dIgA testing as described herein as a valuable addition to antigen and nucleic acid testing. While antigen testing detects active infection, dIgA levels grow as viral antigen levels decline. dIgA positive result indicates a recent infection however, combined with antigen or molecular testing provides a method for detecting active and recent (including non-active) infection. COVID-19 patients with detectable viral loads by polymerase chain reaction also tested positive for COVID-19 antigen on Day 0 or 3, then tested negative at day 14 when viral loads subsided. An antibody sample collected at day 28 shows the presence of dimeric IgA allowing backward contact tracing of SARS-COV-2 infections in cases where antigen tests and nucleic acid amplification will be negative. The first and last of these graphs shows examples of how dIgA disappears by 100 days post infection. Therefore, detection of dIgA in plasma is likely to be a result of an infection within the past 100 days.

FIG. 42 is a graphical representation of results showing the benefit of a combined test by illustrating examples where antigen tests returned a negative result but dIgA was detectable. Combination assays are provided as further described herein.

FIG. 43 is a graphical representation of experimental results showing examples of the generation of dIgA following vaccination with either an mRNA (Pfizer) or viral vectored adenoviral (AstraZeneca) SARS-COV-2 vaccine. Each graph represents an individual's immune response after vaccination. The presence of SARS-COV-2 receptor binding domain specific dimeric IgA in plasma was monitored either using a lateral flow assay (LFA) for dimeric IgA (circles), or an ELISA assay for the detection of dimeric IgA (solid squares), or an ELISA assay for the presence of SARS-COV-2 receptor binding domain specific IgG (open squares) as a measure of seroconversion to vaccine overall. The results show that a majority of individuals (61.3%) do not produce dimeric IgA following vaccination. This contrasts to data shown in FIG. 34A where 100% of samples tested positive for dIgA at day 11 post infection. The cut-off for dimeric IgA in LFA is 400 axxin units meaning any value of 400 or above is considered positive for dimeric IgA to SARS-COV-2. The cut-off for dimeric IgA in ELISA is 0.25 meaning that any value above 0.25 is positive for dimeric IgA to SARS-COV-2. The cut off for IgG is 0.5 meaning that any value above 0.5 is considered positive for the presence of IgG to SARS-COV-2.

FIG. 44 illustrates results showing the maximal dIgA individual responses to vaccines. The graph shows the maximal levels of dIgA detected in those vaccine recipients who demonstrated a detectable dIgA response in either an LFA (n=7) or ELISA assay (n=11) among the 31 people that received SARS-COV-2 vaccines. The range of responses as measured in LFA was between 400 and 8710 units with a median of 1990 units. The range of dIgA levels as measured in ELISA was between 0.25 and 3.6 with a median of 1.3.

FIG. 45 is a graphical representation of results showing anti-measles antigen dIgA detection (a) shows anti-measles virus lysate (VL) dIgA is detectable in samples with confirmed acute measles infection and is comparable to the current gold standard IgM (b) with no detectable cross-reactivity in acute rubella, parvo or dengue samples and very low background reactivity from negative controls (samples with provisional diagnosis of measles/rubella but confirmed IgM negative and blood donors). Anti-measles VL dIgA has low correlation to IgM response (c), suggesting it is an independent biomarker with likely a higher signal-to-cut off than anti-measles VL IgM, as shown in pairwise comparison of dIgA and IgM and mean of difference plot for the majority of acute measles samples (d). ****, p value of <0.0001.

FIG. 46 is a graphical representation of results illustrating utility of present invention in non-human species (a) shows that RBD-specific dIgA is elevated in serum samples of two out of four ferrets experimentally infected with SARS-COV-2 (VIC01, 4.64×104 TCID50) by intranasal inoculation (Final bleed=T). Serum was collected from each ferret prior to challenge (Prebleed=P). FIG. 46 (b) shows dIgA in ferret serum captured using various concentrations of CSC reagent (4, 8, and 12 μg/mL) from various dilutions of serum (1 in 25, 1 in 50, 1 in 100 and 1 in 200). Ferret 22 further confirms the elevated presence of RBD-specific dIgA in the terminal bleed (22-T) sample compared to the pre-bleed (22-P) sample. Mean±standard error plotted using Microsoft excel.

FIG. 47 illustrates results of the dIgA assay adapted to a bead-based Luminex® assay employing a fluorescent bead platform for the detection of dimeric IgA. Serial dilutions of monoclonal dimeric IgA specific to an epitope within the RBD of SARS-COV-2, or the same antibody expressed as a monoclonal moneric IgA1 or IgA species. Dilutions of antibodies were added to RBD or whole Spike protein coupled to beads, bound by CSC and detected with anti-SC.

FIG. 48 shows an example of the use a bead-based dIgA Luminex® assay that specifically detects dIgA against SARS-COV-2 antigens in plasma allows measurement of the production of dimeric IgA following infection with SARS-COV-2 or vaccination with SARS-COV-2 antigens.

FIG. 49 shows the utilisation of the dIgA Luminex assay in measuring dIgA responses towards SARS-COV-2 antigens Spike and RBD in both healthy control (HC) individuals (n=252) and convalescent plasma from individuals with confirmed SARS-COV-2 infection (recovered) (n=84).

FIG. 50 shows graphically how IgG antibodies have been converted to IgA or dimeric IgA and retain or show improved neutralizing activity against both ancestral and variants of SARS-COV-2. Examples are given where monoclonal antibodies originally isolated and characterised as IgG isotypes including CB6, B38, and MAb 222, have been converted into IgA1 or IgA2 isotypes and expressed as either monomeric IgA1 or IgA2, or expressed as dimeric IgA1 and IgA2. In the case of MAb222, which is a broadly neutralizing antibody, reactivity was also retained against the highly neutralization resistant Beta variant. In the case of CB6, conversion into dIgA2 resulted in a 2.5 fold improvement compared with IgG version in IC50 against the Hu-1 isolate (A) and a similar level of reactivity of IgG against the Delta variant (B). In the case of B38, conversion into IgA or dimeric IgA forms improved the activity of this MAb against the ancestral Hu-1 isolate 2-10 fold relative to IgG form (C). In the case of broadly reactive MAb 222, dIgA1 retained a similar level of activity against the Hu-1 isolate (E) the delta isolate (F) and the Beta isolate (G) compared to IgG. The IC50 data is summarised in a heat map in (H). The lower the value the more potent the antibody activity and is shown in μg/ml.

### DISCUSSION OF EMBODIMENTS

Unless defined otherwise, all technical and scientific terms used herein have the same meanings as commonly understood by one of ordinary skill in the art to which this disclosure belongs. Any materials and methods similar or equivalent to those described herein can be used to practice or test the present disclosure. Practitioners are particularly directed to William E Paul ed Fundamental Immunology Fifth-Seventh Edition 2003-present, and Ausubel et al., Current Protocols in Molecular Biology, Supplement 47, John Wiley & Sons, New York, 1999; Colowick and Kaplan, eds., Methods In Enzymology, Academic Press, Inc.; Weir and Blackwell, eds., Handbook of Experimental Immunology, Vols. I-IV, Blackwell Scientific Publications, 1986; for definitions and terms of the art and other methods known to the person skilled in the art. The structure and function of IgA and dIgA are described in Woof and Russel, 2011. See also Sousa-Pereira Antibodies 2019.

IgG antibodies commonly persist for life and blood levels may indicate either current or past infection with a pathogenic organism. For chronic infections such as the human immunodeficiency virus (HIV) where patients do not spontaneously clear the virus, detection of IgG-class antibodies is diagnostic for infection, whereas for others such as hepatitis C virus (HCV) where a proportion of patients do clear the virus either spontaneously or following treatment, the detection of antigen-specific IgG is not diagnostic of current or ongoing infection. IgG-class antibodies are primarily responsible for antibody-mediated immunity within the plasma compartment of the body.

IgA-class antibodies have also been used to aid diagnosis of infections however little attempt has been made to tease out the differential roles of dIgA and IgA in this context, mainly because the common detection reagents for IgA detect both dIgA and monomeric IgA. The term “IgA” or “total IgA” as used herein refers collectively to both subclasses (IgA1 or IgA2) and subtypes “m” “d” or “s” of IgA (overall there are six subtypes dIgA, mIgA, SIgA, dIgA2, mIgA2 and SIgA2 which fall within the two subclasses). “m” refers to monomeric, “d” refers to dimeric, “s” refers to secretory.

The term “dIgA” as used herein refers to dimeric or higher polymeric forms of IgA which are bound together including by a J-chain. In humans and higher primates, the dIgA is only a minor fraction of the total IgA, with monomeric IgA (mIgA) representing around 90% of the total IgA, and dimeric or higher polymeric forms of IgA representing around 10% of the total IgA. SIgA comprises secretory component and is found mainly at the mucosa. In one embodiment, reference to dIgA does not include polymeric forms of dIgA which contain the secretory component such as SIgA. Reference to polymeric IgA includes dIgA and SIgA and alternative multimeric forms thereof that may be circulating, at mucosal sites or produced synthetically or recombinantly. In one embodiment a dIgA or derivative comprises one or more dIgA constant regions. A “proportion” of pathogen specific dIgA that is neutralizing includes at least 10%, 20%30%, 40%, 50% or more of pathogen specific dIgA antibodies being neutralizing or recognising antigens/epitope that are important for infectivity/pathogenicity at the mucosa.

The term “SIgA” as used herein refers to secretory IgA typically found at the apical mucosal surface or within the lumen of the mucosal surface such as the liquid volume of the upper- or lower-respiratory tracts or the gastrointestinal tract. dIgA binds to polymeric Ig receptor (pIgR) at the basolateral surface and is transcytosed and released at the apical surface bound to secretory component which has cleaved off from pIgR. The interaction between dIgA and pIgR is dependent on the presence of the J-chain.

Reference to “subject” or “subjects” includes a subject or individual to be treated or tested and a donor for plasma or immunoglobulin collection, or a human or non-human animal used to generate immune sera or antibodies. The term therefore includes humans and a wide range of mammalian, non-mammalian, avian or other animals including wild and domesticated animals, horses, camelids, rabbits, rodents, guinea pigs, dogs, pets, pests and potential vehicles for emerging infectious diseases. In one embodiment, the subject is a mammalian or avian animal species. In an embodiment, the mammal is a human. In relation to subjects, the subjects may be at risk of, suspected of or diagnosed with an infectious disease or pathogen, condition, infection or exposure to a venom or toxin.

In one embodiment, the pathogen (infectious organism) is a coronavirus such as SARS-COV-2, SARS-COV, MERS, or a potentially pandemic coronavirus. Coronaviruses are known to cause disease in humans and animals. Among these, four human coronaviruses 229E, NL63, OC43 and HKU1 typically infect the upper respiratory tract and cause minor symptoms. However, three coronaviruses, (severe acute respiratory syndrome coronavirus (SARS-COV), Middle East respiratory syndrome coronavirus (MERS-COV) and SARS-CoV-2 appear to replicate more broadly and can frequently cause more serious symptoms. In one embodiment, the CoV antigens are selected from Spike (S) protein, nucleocapsid (N) protein, membrane (M) protein, and envelope (E) protein. It must be born in mind that pathogenic organisms are by definition capable of causing severe symptoms, and this is particularly so during an epidemic or pandemic, not all the population may respond to infection with severe symptoms and indeed many may be asymptomatic, at least initially. In order to characterise a population and the spread of the pathogen within the community, it is important to be able to assess infection in asymptomatic but infected and therefore infective subjects at an early stage post infection.

Suitable antigens of SARS-COV-2 include the Spike protein and the receptor binding domain (RBD) thereof within the S1. In one non-limiting embodiment, the coronavirus antigen is Spike protein, a sub-domain or antigenic/antibody binding portion of the Spike protein e.g., comprising all or part of the S1 subunit or the S2 subunit. The S1 subunit comprises, inter alia, the N-terminal domain and the RBD which are important for antibody binding. In one embodiment, the antigen is selected from one or two or three or four or more of Spike (S) protein or its RBD, nucleocapsid (N) protein, membrane (M) protein, and envelope (E) protein. The methods of the present application related to measuring levels of pathogen specific dIgA may use non-neutralizing antigens (antigens that do not engender a neutralizing immune response). Where it is important to identify functional or neutralizing pathogen specific dIgA then antigens are selected that are neutralizing antigens. Typically, neutralizing antibodies are directed against components of the pathogen that interact with the host, such as the cellular receptor that mediates cell invasion (in intracellular pathogens). Thus, in one embodiment, neutralizing antigens will comprise the receptor binding domain (RBD) of the pathogen. Pathogenic antigens and antigens that are the target of neutralizing antibodies are known in the art. The RBD in the S1 domain of SARS-COV, the RBD in the HAI subunit of influenza A virus hemagglutinin, and the RBD within the Hendra virus G glycoprotein are illustrative examples.

The term “Coronaviridae”, refers to viruses known by the common name of “Coronavirus” or “CoV” which are enveloped, positive sense, single-stranded RNA viruses. There are two subfamilies of Coronaviridae, Letovirinae and Orthocoronavirinae. In one embodiment, the CoV is selected from the genera Alphacoronavirus (alphaCoV). Betacoronavirus (betaCoV), Gammacoronavirus (gammaCoV) and Deltacoronavirus (deltaCoV). In one embodiment, the alphaCoV is selected from coronavirus 229E (HCoV-229E), human coronavirus NL63 (HCoV-NL63), transmissible gastroenteritis virus (TGEV), porcine epidemic diarrhea virus (PEDV), feline infectious peritonitis virus (FIPV) and canine coronavirus (CCoV). In one embodiment, the betaCoV is selected from human coronavirus HKU1 (HCoV-HKU1), Human coronavirus OC43 (HCoV-OC43), Severe acute respiratory syndrome-related coronavirus (SARS-COV), Severe acute respiratory syndrome-related coronavirus-2 (SARS-COV-2), Middle-East respiratory syndrome-related coronavirus (MERS-COV), murine hepatitis virus (MHV) and/or bovine coronavirus (BCoV). In one embodiment, the CoV is capable of infecting a human. In one embodiment, the CoV capable of infecting a human is selected from: SARS-COV-2, HCoV-OC43, HCOV—HKU1, HCOV-229E, HCoV-NL63, SARS-COV, and MERS-COV or a subtype of variant thereof. In one embodiment, the CoV is SARS-COV-2 or a subtype or variant thereof. In one embodiment, the SARS-COV-2 is SARS-COV-2 subtype L as described in Tang et al., 2020. In one embodiment, the SARS-COV-2 is SARS-COV-2 subtype S as described in Tang et al., 2020. In an embodiment, SARS-COV-2 is SARS-COV-2 hCoV-19/Australia/VIC01/2020. In one embodiment, SARS-COV-2 comprises the sequences as described in NCBI Reference Sequence: NC_045512.2 (ancestral Hu-1). In one embodiment, SARS-COV-2 comprises the sequence as described in GenBank: MN908947.3 or a variant thereof. Examples of SARS-CoV-2 variants are described, for example, in Shen et al., 2020, Tang et al., 2020, Phan et al., 2020 and Khan et al 2020. Foster et al (2020) have found 3 variants, A, B and C, based on genomic analysis. In some embodiments, the SARS-COV-2 is SARS-COV-2 variant A. In some embodiments, the SARS-COV-2 is SARS-COV-2 variant B. In some embodiments, the SARS-COV-2 is SARS-COV-2 variant C.

In one embodiment, the variant is at least 90% identical to the parental sequence. In one embodiment, the variant is at least 92% identical to the parental sequence. In one embodiment, the variant is at least 93% identical to the parental sequence. In one embodiment, the variant is at least 94% identical to the parental sequence. In one embodiment, the variant is at least 95% identical to the parental sequence. In one embodiment, the variant is at least 96% identical to the parental sequence. In one embodiment, the variant is at least 97% identical to the parental sequence. In one embodiment, the variant is at least 98% identical to the parental sequence. In one embodiment, the variant is at least 99% identical to the parental sequence. In some embodiments, the parental strain is SARS-COV-2 hCoV-19/Australia/VIC01/2020. In some embodiment, the parental strain is BetaCoV/ancestral Hu-1//WIV04/2019 (NC_045512.2).

CoV infections cause can cause respiratory, enteric, hepatic, and neurological diseases in different animal species, including camels, cattle, cats, and bats. CoV can be transmitted from one individual to another through contact of viral droplets with mucosa. Typically, viral droplets are airborne and inhaled via the respiratory tract including the nasal airway. Typically, the individual is a human individual. In some embodiments, the individual is a live stock or domestic animal. Typically, during an infection, CoV can be found in the upper respiratory tract, for example the nasal passages. In some examples, CoV can be found in the lower respiratory tract, for example the bronchi and/or alveoli.

In an embodiment, a CoV infection causes one or more symptoms selected from one or more of: fever, cough, sore throat, shortness of breath, viral shedding respiratory insufficiency, runny nose, nasal congestion, malaise, bronchitis, headache, muscle pain, dyspnea, moderate pneumonia, severe pneumonia, acute respiratory distress syndrome (ARDS). In an embodiment, the ARDS is selected from mild ARDS (defined as 200 mmHg <PaO2/FiO2≤300 mmHg), moderate ARDS (defined as 100 mmHg <PaO2/FiO2≤200 mmHg) and severe ARDS (defined as PaO2/FiO2≤100 mmHg). In an embodiment, a SARS-COV-2 infection can cause one or more symptoms selected from one or more of: fever, cough, sore throat, shortness of breath, viral shedding, respiratory insufficiency, runny nose, nasal congestion, malaise, bronchitis, headache, muscle pain, dyspnea, moderate pneumonia, severe pneumonia, acute respiratory distress syndrome (ARDS).

In an embodiment, the infection by a pathogen causes no symptoms in some members of the population (an individual is asymptomatic). In an embodiment, the CoV infection cause no symptoms in some members of the population (an individual is asymptomatic).

The coronavirus spike (S) glycoprotein is a type I transmembrane glycoprotein that produces recognizable crown-like spike structures on the virus surface. The receptor-binding domain (RBD) of the S protein facilitates viral entry into human cells through human angiotensin-converting enzyme 2 (ACE2) receptor binding. Due to similar sequences between SARS-COV and SARS-COV2 spike proteins antibodies may bind both antigens. Thus, the terms antigen-specific or pathogen-specific include highly related pathogen/antigens.

Reference to an infectious organism or agent or pathogenic organism or agent and an antigen derived there from is to be broadly construed. In some embodiments, the infection is caused by an infectious agent or microbe selected from a bacterium, fungus, virus, algae, parasite, prion, oomycetes, slime, moulds, nematodes, mycoplasma and the like. In one embodiment, the infectious or pathogenic organism is selected from one or more of the following orders, genera or species: Acinetobacter, Actinobacillus, Actinomycetes, Actinomyces, Aeromonas, Bacillus, Bacteroides, Bordetella, Borrelia, Brucella, Burkholderia, Campylobacter, Citrobacter, Clostridium, Corynebacterium, Enterobacter, Enterococcus, Erysipelothrix, Escherichia, Francisella, Haemophilus, Helicobacter, Klebsiella, Legionella, Leptospira, Listeria, Micrococcus, Moraxella, Morganella, Mycobacterium (tuberculosis), Nocardia, Neisseria, Pasteurella, Plesiomonas, Propionibacterium, Proteus, Providencia, Pseudomonas, Rhodococcus, Salmonella, Serratia, Shigella, Staphylococcus, Stenotrophomonas, Streptococcus, Treponema, Vibrio (cholera) and Yersinia (plague), Adenoviridae, African swine fever-like viruses, Arenaviridae (such as viral hemorrhagic fevers, Lassa fever), Astroviridae (astroviruses) Bunyaviridae (La Crosse), Calicivirid (Norovirus), Coronaviridae (Coronavirus), Filoviridae (such as Ebola virus, Marburg virus), Parvoviridae (B 19 virus), Flaviviridae (such as hepatitis C virus, Dengue viruses), Hepadnaviridae (such as hepatitis B virus, Deltavirus), Herpesviridae (herpes simplex virus, varicella zoster virus), Orthomyxoviridae (influenza virus) Papovaviridae (papilloma virus) Paramyxoviridae (such as human parainfluenza viruses, mumps virus, measles virus, human respiratory syncytial virus), Nipah virus, Hendra virus, Picornaviridae (common cold virus), Poxviridae (small pox virus, orf virus, monkey poxvirus) Reoviridae (rotavirus) Retroviridae (human immunodeficiency virus) Paroviridae (paroviruses) Papillomaviridae, (papillomaviruses) alphaviruses and Rhabdoviridae (rabies virus), Trypanosoma, Leishmania, Giardia, Trichomonas, Entamoeba, Naegleria, Acanthamoeba, Plasmodium, Toxoplasma, Cryptosporidium, Isospora, Balantidium, Schistosoma, Echinostoma, Fasciolopsis, Clonorchis, Fasciola, Opisthorchis and Paragonimus, Pseudophyllidea (e.g., Diphyllobothriuni) and Cyclophyllidea (e.g., Taenia). Pathogenic nematodes include species from the orders; Rhabditida (e.g., Strongyloides), Strongyliaa (e.g., Ancylostoma), Ascarida (e.g., Ascaris, Toxocara), Spirurida (e.g., Dracunculus, Brugia, Onchocerca, Wucheria), and Adenophorea (e.g., Trichuris and Trichinella), further groups include Prototheca and Ptiesteria, Absidia, Aspergillus, Blastomyces, Candida (yeast), Cladophialophera, Coccidioides, Cryptococcus, Cunninghamella, Fusarium, Histoplasma, Madurella, Malassezia, Microsporum, Mucor, Paecilomyces, Paracoccidioides, Penicillium, Pneumocystis, Pseudallescheria, Rhyzopterus, Rhodotorula, Scedosporium, Sporothrix, Trichophyton and Trichosporon. For the avoidance of doubt the infectious agent may be a known pathogen, an emerging or re-emerging pathogen or an organism which has never previously been identified as a pathogen in a particular subject. In some embodiments, the infectious agent is resistant to usual medicaments, e.g., resistant to at least one anti-infective agent. In some embodiments, the infectious agent is resistant to multiple anti-infective agents (e.g., multi-drug resistant). The term pathogenic antigen refers to antigens of interest derived from the above organism but also encompasses toxins or venoms against which a functional antibody immune response is highly sought for anti-toxin and anti-venom therapeutics. In one embodiment, the pathogen is a pathogen that induces a muscosal immune response. In one embodiment, the pathogen is an organism capable of transmitting as an epidemic or pandemic.

In one embodiment the pathogenic antigen is a venom. Antivenom, for example, is currently the only treatment for bites by venomous Australian snakes. Prior to the availability of antivenom, death ensued in approximately 45% of tiger snake envenomations and more than 90% of taipan envenomations.

In one embodiment, the infectious organism is a virus and includes influenza viruses, respiratory syncytial virus, varicella zoster virus, Epstein Barr virus. In one embodiment, infectious organism is a bacterial pathogen.

The present application discloses and enables methods and uses that are broadly applicable to pathogens and pathogenic antigens as described herein above. Preference is given in some embodiments to antigens that mediate pathogen life cycle specific activities, such as invasion (e.g. envelope or spike protein), or down regulate host responses to invading organisms. Thus, antigens frequently used in vaccines are those against which functional antibody responses are generated such as host surface or host molecule binding antigens, receptor binding antigens, receptor antigens etc.

A suitable “biological sample” for the methods and kits as described herein includes any sample containing or suspected of containing antibodies for detection including, but not limited to, biological fluids e.g. whole blood or a fraction thereof, a blood product, plasma, a mucosal surface sample such as serum, saliva, nasal swab, throat swab, respiratory swab, nasal scrapings, nasal washings, respiratory tract washing, lung washings, gut samples, faeces or gingivo creviscular fluid. In one particularly advantageous embodiment, the sample is whole blood or a part or derivative thereof comprising circulating antibodies. In another particularly convenient embodiment, the sample is a mucosal surface sample such as serum, saliva, nasal swab, throat swab, respiratory swab, nasal scrapings, nasal washings, respiratory tract washing, lung washings, gut samples, faeces or gingivo creviscular fluid comprising circulating antibodies or combinations thereof. In one embodiment, the sample is any sample containing dIgA antibodies. In one embodiment, the sample is purified or partially purified. In an embodiment the sample is plasma. In an embodiment, the sample is serum. In an embodiment, biological samples are collected from a subject at two more time points. In an embodiment, the sample is stored for a period of time at about 4° C., at about 15° C. or about 24° C. before use. In an embodiment, the sample is dried, freeze dried or snap frozen. Non-limiting examples of a blood product include whole blood, serum, plasma, the like, and a combination thereof. A blood product may be devoid of cells, or may include cells (e.g., red blood cells, platelets and/or lymphocytes). In one embodiment, one sample is whole blood of a fraction thereof such as plasma and another sample to be tested in the same assay is a mucosal sample such as saliva or nasal samples.

Reference to a “blood sample” includes a plasma sample or a serum sample or other blood part comprising antibodies derived from a blood sample.

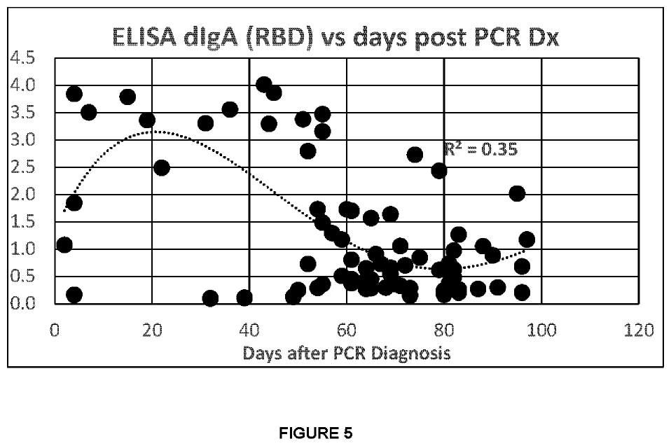

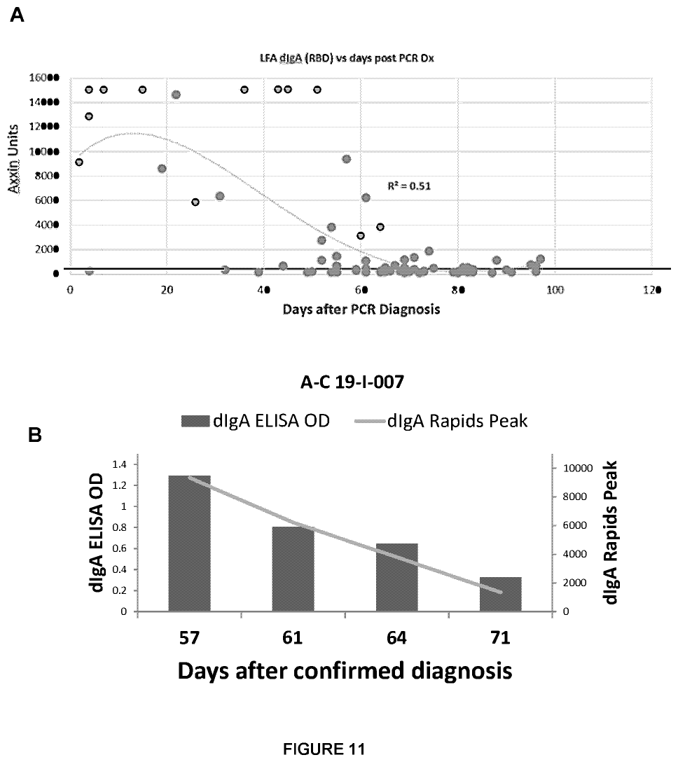

“Start” is an estimation of the date or time period of infection or vaccination, it may be known, or it may be estimated from the time of nucleic acid or antigen based diagnosis of infection, time of symptom onset reported by the subject or other estimate. Knowing the Start, and the reliable trajectory of dIgA levels in the circulation as described herein including its typical precipitous decline at 2-3 months (see FIGS. 5 and 11), the skilled person can estimate the magnitude of the mucosal immune response by the subject. Similarly, where the Start is unknown (often the issue in contract tracing and work place screening), the level of dIgA, and the reliable trajectory of dIgA levels in the circulation as described herein, can be used by the skilled person to estimate the Start. The method allows for the inclusion or exclusion of subjects during contact tracing processes. At its most basic level, a subject with a level of dIgA, such as pathogen specific dIgA, is not likely to have been infected more than about three months previously because dIgA disappears precipitously from the circulation at 2-3 months. In one embodiment, the Start is a known time of infection or vaccination, or is estimated from a known time of symptom onset/positive test result for the presence of pathogen. It should be understood that inclusion of a Start in the subject methods provides a more quantitative assessment, specifically a more precise estimate of the time since infection. However, the detection of a level of pathogen specific circulatory dIgA without reference to a Start still indicates that the subject has seroconverted to the production of dIgA, which coupled with the finding herein that levels of circulatory pathogen specific dIgA are strongly correlated with the levels of neutralizing dIgA, indicates the subject can produce a functional mucosal immune response to the pathogen. In one embodiment, the detection of a level of Spike specific circulatory dIgA without reference to a Start still indicates that the subject infected with SARS-COV-2 has seroconverted to the production of dIgA, which coupled with the finding herein that levels of circulatory Spike specific dIgA are strongly correlated with the levels of neutralizing dIgA, indicates the subject can produce a functional mucosal immune response to the pathogen.

Reference to “1 to 3” months provides the time period from infection during which a subject may seroconvert to producing pathogen specific dIgA that can be detected in the blood i.e., circulatory dIgA. As determined herein from longitudinal studies, dIgA levels are rapidly lost from the blood circulatory system at about 1 to 3 months from infection.

A “level” of dIgA in the circulation indicates that the subject has circulating dIgA above a threshold. In terms of, for illustration, contact tracing or work place screening, this means that the subject was infected approximately less than 1-3 months.

A “high level”, “medium level” or ‘low level” of dIgA or antigen-specific dIgA may be relative to a subject, population or control level. It is also referenced to a one-off measurment or a series of measuments over time (say over 11 to100 days). For example, when assessing plasma or serum for convalescent therapy it is important to identify the highest available sources of dIgA within the available population of donors. Thus, what is high in one population or at one time, may be categorized as medium in another population or at another time. A high level for plasma therapy may be in the top 5%, 10%, 15%, 20%, 25%, or 30% of samples. A low level may be in the bottom 50% of dIgA positive samples. A medium level may be between the low and the high. High level plasma is also useful for purifying polyspecific or monospecific dIgA to produce a composition comprising dIgA as the active ingredient, however, medium level plasma may also be employed for this purpose. In one embodiment, the active ingredient is able to engender a functional mucosal immune response. In one embodiment, the polyspecific and the monospecific dIgA binds to the Spike protein or a spike antigen.

A “magnitude of the immune response” is determined from an individual test result or from a cohort of individuals, or from several test results from different blood samples from the same person or cohort. In one embodiment, the immune response is a mucosal immune response to a pathogen. In one embodiment, the immune response is a mucosal neutralizing immune response to a pathogen. dIgA is trancytosed at the mucosa and therefore pathogen neutralization may take place extracellularly or intracellularly. In one embodiment, a positive circulatory pathogen specific dIgA test result indicates the ability of the subject to mount a mucosal neutralizing immune response to a pathogen and that the subject is mounting a mucosal neutralizing immune response to a pathogen. In another embodiment, the level of circulatory pathogen specific dIgA in a test result when the start time period is known allows the skilled person to quantify the magnitude of the mucosal immune response that the subject has or can produced. In particular, the dIgA level is compared, for example, to the area under curve or equivalent value, when time since infection/start is compared to the dIgA level at different times following a predictable trajectory, and indicates the total dIgA response to the pathogen. This may be quantified by the methods disclosed herein.

“Functional” antibody or mucosal immune response refers herein to the ability of dIgA antibodies that are produced by circulating B-cells and present in circulating blood during the early phase of pathogen infection, and/or those that are produced by B-cells which have migrated rapidly to the mucosa approximately 1 to 3 or 2 to 3 months after start and are not present in circulating blood, to reach the mucosa (either via the circulation or directly from resident mucosal B-cell production) where they are processed to SIgA and effect a mucosal immune response involving antigen/pathogen binding and a reduced infection. Furthermore, as determined herein, a proportion of circulating dIgA is neutralizing and it is proposed herein that this ability is maintained at the mucosa. Similarly, it is proposed herein that a high level of circulatory dIgA overtime is a marker of a high level of circulating dIgA-producing B-cells which will later migrate to the mucosa to provide a functional and durable mucosal immune response at the mucosa. While, IgG which is produced later in infection includes neutralizing antibodies, these antibodies are not likely in accordance with the present invention to form a substantial part of the mucosal immune response, in part because IgG antibodies are not transported by active receptor-mediated transport into the relevant mucosal tissues (respiratory and gastrointestinal) and instead rely on passive transfer across the mucosal epithelium, resulting in much higher relative concentrations of dIgA (as SIgA) compared to IgG in the mucosal compartment. Measurement of neutralization ability therefore is not necessarily a marker for mucosal effects unless the antibody subclass is dIgA. Confirmatory assays for functional antibodies includes neutralization assays. Other assays include protein binding or mucin binding assay or the like.

### Screening Convalescent Plasma

Convalescent plasma therapy is known in the art and suitable procedures are established. The present application describes a method for screening convalescent subjects to determine their suitability as plasma donors. In particular, pursuant to the longitudinal serology studies and neutralization assays described herein, a screening method directed to identifying dIgA levels is provided.

Not all convalescent plasma is suitable for convalescent plasma therapy as the individual immune response may vary. In particular, not all SARS COV-2 or pathogen infected people will produce detectable dIgA or more than very low levels of dIgA. As determined herein, a convalescent subject is not likely to have levels of circulating dIgA after 2 to 3 months from infection. It is proposed to identify individuals with the highest percentile levels of dIgA, say the top 30%, 25%, 20%, 15%, 10%, or 5% for convalescent plasma therapy. In one embodiment, the highest levels are prioritised for therapeutic use in patients with the worst symptoms, such as lung, gut, or other mucosal surface involvement.

The prior art measures blood IgG which is not strongly functional at mucosal surfaces, because very little of the administered IgG will be transported to the mucosa. In accordance with the present invention monitoring blood dIgA levels provides a window into the magnitude of the antigen specific mucosal immune response. It is proposed that high titre dIgA as plasma or in more concentrated form when administered to a subject is delivered to the mucosal surface, lungs, GI tract etc where it can functionally reduce the infection at the sites in the body where the pathogen is replicating. In addition, dIgA may interact with and neutralize virus within the cells of these mucosal surfaces during the process of transcytosis.

In one embodiment the present application provides a method for identifying or stratifying a convalescent human subject for prioritisation as a blood or plasma donor for convalescent plasma/immunoglobulin therapy, said method comprising assessing, measuring or determining a level or presence of pathogen specific dIgA in a blood sample from the subject who has been infected with the pathogen and is no longer considered infectious. The identification of a level of dIgA or pathogen specific dIgA in the blood sample indicates the production by the subject of pathogen specific functional antibodies and the suitability of the subject for prioritisation as a blood or plasma donor for therapeutic or prophylactic convalescent plasma administration against the pathogen or for the isolation of B-cells or antibody-producing genes for the recombinant production of such antibodies. In one embodiment, administration is systemic, via for example, by intravenous, subcutaneous or intramuscular delivery.

Illustrative ELISA and rapid point of care methods are described in the examples and Figures.

In one embodiment, the method comprises measuring or determining the level of pathogen specific total immunoglobulin in the sample.

As determined herein, a proportion of the pathogen specific circulatory dIgA are functional that is they contribute to a functional mucosal immune response, as may be determined by assays such as neutralization assays. Thus “functional” includes the ability of dIgA and/or its processed (transcytosed) forms (SIgA) to directly or indirectly bind to an infectious agent thereby preventing entry of an infectious agent or binding toxins or venoms to ameliorate their effect on host tissues and the host. In one embodiment the proportion is at least 1%, 5%, 10%, 20%, 30%, 40%, or at least 50%. As discussed, it is proposed that dIgA moves to the mucosa where it is expressed as SIgA which substantially effects the functional mucosal immune response.

As determined herein, a proportion of the CoV-2 Spike or Spike RBD specific dIgA are functionally neutralizing. In one embodiment the proportion is at least 2%, 5%, 10%, 20%, 30%, 40%, or at least 50% or more.

In one embodiment, a proportion of Spike RBD specific dIgA are functionally neutralizing. In one embodiment the proportion is at least 5%, 10%, 20%, 30%, 40%, or at least 50% or at least 60% or at least 70% or more. In one embodiment, the presence of pathogen specific dIgA in the sample indicates the presence of pathogen specific dIgA that are functionally neutralizing.

In one embodiment, the presence of Spike protein or Spike RBD specific dIgA in the sample indicates the presence of pathogen specific SIgA in the subject that are functionally neutralizing.

In one embodiment, the presence of pathogen specific dIgA in the sample indicates the ability of the subject to produce pathogen specific SIgA in response to infection by the pathogen or effective vaccination that are functionally neutralizing.

In one embodiment, the presence of Spike specific dIgA in the sample indicates the presence of pathogen specific SIgA that are functionally neutralizing.

In one embodiment, the presence of Spike RBD specific dIgA in a sample from a convalescent subject indicates the presence of pathogen specific SIgA that are functionally neutralizing.

In one embodiment, the presence of Spike RBD specific dIgA in a sample from a convalescent subject indicates the presence of pathogen specific dIgA that are functionally neutralizing.

In one embodiment, the sample is assessed for the presence of a level of dIgA or pathogen specific dIgA. In one embodiment, the sample is assessed for the presence of a high level of dIgA or a high level of pathogen specific dIgA. In one embodiment, the sample is assessed for the presence of a medium level of dIgA or a medium level of pathogen specific dIgA. In one embodiment, the sample is assessed for the presence of a low level of dIgA or a low level of pathogen specific dIgA. In the simplest form of this embodiment, there is no determination of a start time or the magnitude of the total dIgA response. That is the method comprises screening a potential donor for whether they can be sampled immediately because they have a high or even medium level of antigen specific dIgA at the time of screening.

In one embodiment, the attributed or determined level of pathogen specific dIgA (such as present, absent, low, medium or high) is determined by reference and is relative to one or more pre-determined values, optionally from one or more populations or cohorts. Thus, a level high could reference the level of dIgA in the top 20-30% percentile of subjects tested in a day/week/month optionally above a threshold level. Medium could be the next 50 to 80%, low could be below 50%. In one embodiment, the attributed level is determined in part by the exigencies of the situation. In other embodiment, threshold levels of for example, low, medium and high dIgA levels are established which provide useful information on an ongoing basis.

In one embodiment, when dIgA is detected in convalescent serum, levels may be compared with a reference time line to determine a time since start (eg, time since infection) or to provide an even more quantitative estimate of the capacity of the subject to mount a functional mucosal immune response to the pathogen. In one embodiment, this is useful in determining when the subject is likely to go dIgA negative and therefore, for example, how soon plasma should be sampled from the subject and whether multiple plasma collections may be sampled within an appropriate time frame while dIgA positive.

In one embodiment, this further aspect of the method comprises comparing the dIgA antibody level to a pre-determined reference stratum value to indicate a time since a start time (for example, time from IST-infection/symptoms/testing for presence of pathogen). In one embodiment, the time is a start range or window based on information provided/contact tracing etc.

In one embodiment, the reference strata value is obtained from the pre-determined level or change in the level of pathogen specific dIgA antibody over time since a start time, from the blood from reference subjects. One useful value in this context is the area-under-the-curve (AUC) from a plot of dIgA levels against time since the start of infection or a surrogate marker of same.

In some embodiments, the start time is determined from the sample so as to prioritise subject as blood/plasma donors. Thus, subjects may be identified as having a start time of 10 to 40 days, indicating blood/plasma may be taken within the next two-three weeks and a second or further blood/plasma sample may also be taken thereafter, whereas a start time of 40 to 80 days indicates that blood/plasma should be taken as soon as possible. Alternatively, where the start time is known, the magnitude of the mucosal immune response may be assessed. Screening is repeated at each relevant time point.

In one embodiment, a method is practised for assessing the time since infection by a pathogen, the method comprising (i) measuring or determining a pathogen-specific dIgA antibody level in a biological sample from a subject and (ii) comparing the antibody level from (i) to a pre-determined reference stratum value/s to indicate a time since infection/start time.

In one embodiment, the level of total Ig or IgG in the sample is determined to facilitate identification of time since Start, as described further herein.

In one embodiment, the reference strata value is obtained from the pre-determined level or change in the level of pathogen specific dIgA antibody over time since a start time from the blood in reference subjects.

In one embodiment, the time since infection determined from the sample is one or more of: a recent-infection stratum i.e., about 40 to 80 days since infection; or a past-infection stratum, i.e., more than 80 days since infection.

In another embodiment, a method is practised for assessing the magnitude of the immune response to a pathogen where the start time is known, the method comprising (i) measuring or determining a pathogen-specific dIgA antibody level in a biological sample from a subject and (ii) comparing the antibody level from (i) to a pre-determined reference stratum value to indicate the magnitude of the immune response.

The method comprises contacting a biological sample comprising antibodies such as blood or a fraction thereof from a subject with a dIgA binding agent to allow formation of a complex between pathogen specific dIgA and the dIgA binding agent and determining the level of pathogen specific dIgA therefrom.

In one embodiment, the dIgA binding agent is specific to dIgA. In one embodiment, the dIgA binding agent is pIgR or a chimeric pIgR or modified form of either wherein the pIgR binds dIgA and substantially fails to bind IgM. In one embodiment, the pIgR is CSC, being human pIgR with domain 1 from rabbit pIgR.

In one embodiment, the method is an ELISA-based method or a rapid point of care-based method. Many suitable general assay formats are known in the art.

### Screening for Epidemiological Information-Contact Tracing, Work Place Screening Etc

In one embodiment the present application provides a method for immune response surveillance or contact tracing, said method comprising measuring or determining a level of pathogen specific dIgA in a blood sample from the subject, wherein the identification of a level of pathogen specific dIgA in the blood sample indicates a time since infection.

In one non-limiting embodiment, the method comprises (i) measuring or determining a pathogen-specific dIgA antibody level in a blood sample from a subject and (ii) comparing the antibody level from (i) to a pre-determined reference stratum value to indicate a time since infection/start time.

As described herein, the reference strata value/s may be obtained from the pre-determined level or change in the level of pathogen specific dIgA antibody over time since start from the blood of reference subjects. Thus, as surprisingly determined herein, a plot of dIgA levels over time provides a reliable trajectory and the mathematical formula describing the trajectory/curve allows the skilled person to predict other data points on the line including values such as the AUC indicative of the subjects total dIgA based immune response over the period, the time since start or the magnitude of the immune response. Other useful outputs may become apparent that these examples are illustrative.

By way of example, in one embodiment, the time since infection determined from the sample is one or more of:
a) an incident (or contemporaneous) infection stratum i.e., 2 to 10 days since infection;
b) a current infection stratum i.e., 10 to 40 days since infection;
c) a recent-infection stratum i.e., 40 to 80 days since infection;
d) a past-infection stratum, i.e., more than 80 days since infection.

By way of another example, in one embodiment, and using an example of SARS-CoV-2, the time since infection determined from the sample via dIgA and or antigen/molecular screening is one or more of:
a) an incident (current or contemporaneous) infection stratum i.e., 2 to 10 days since infection;
b) a recent (recent past and present infection) infection stratum i.e., 2 to 80 days since infection;
c) or a recent (past)-infection stratum i.e., 11 to 100 days since infection;
d) a past-infection stratum, i.e., more than 100 days since infection.

In one embodiment, the method comprises contacting a biological sample comprising antibodies such as blood or a fraction thereof from a subject with a dIgA binding agent to allow formation of a complex between pathogen specific dIgA and the dIgA binding agent and determining the level of pathogen specific dIgA therefrom.

In one embodiment, the dIgA binding agent is pIgR or a chimeric pIgR or modified form of either wherein the pIgR binds dIgA and substantially fails to bind IgM. In one embodiment, the pIgR is CSC, being human pIgR with domain 1 from rabbit pIgR.

Illustrative ELISA and rapid point of care methods are described in the examples and Figures.

In one embodiment, the method comprising measuring or determining the level of pathogen specific total immunoglobulin in the sample.

### Screening for Vaccine Responses, Immune Surveillance

In another aspect, the present invention provides methods for measuring or determining the presence or magnitude of a dIgA driven neutralizing mucosal immune response in a subject who has been infected with a pathogen or determining the presence or magnitude of a dIgA driven neutralizing mucosal immune response in a subject after vaccine administration.

In one embodiment, an immune response surveillance method is provided for assessing the neutralizing mucosal immune response to a pathogen or vaccine against a pathogen, the method comprising (i) determining the pathogen or vaccine-specific dIgA antibody level in a biological sample from a subject. In one embodiment, the pathogen antigen is a neutralizing antigen. In one embodiment the pathogen specific antigen is a neutralizing antigen. In one embodiment, the antigen is Spike or a part thereof comprising the RBD.

In one embodiment, a method is provided for assessing the neutralizing mucosal immune response to a pathogen or vaccination against a pathogen, the method comprising (i) determining the pathogen or vaccine-specific anti-RBD comprising antigen dIgA antibody level in a blood/plasma/serum sample from a subject.

In one embodiment, the subject has been previously administered a vaccine at a known time point within 1 to 12 weeks and the method comprises (ii) comparing the antibody level to a reference response value/s to indicate the magnitude of the mucosal immune response to the vaccine.

In one embodiment, the reference response value/s is/are obtained from the pre-determined level or change in the level of pathogen specific dIgA antibody over time from the blood in reference subjects. In one embodiment, the reference value is an Area Under the Curve (AUC). In one embodiment, the reference response value/s is/are obtained from the pre-determined level or change in the level of pathogen specific dIgA antibody over time from the blood or mucosal sample in reference subjects. In one embodiment, the reference value is an Area Under the Curve (AUC).

In one embodiment, the assay comprises comparing the pathogen specific dIgA antibody level determined from the assay to a reference response value/s to facilitate determining one or more of (i) the magnitude of the mucosal immune response to the pathogen or vaccine or (ii) the time since Start or start of infection, wherein the reference response value is obtained from the pre-determined level or change in the level of pathogen specific dIgA antibody over time from the blood in reference subjects.

In one embodiment, the assay comprises comparing the pathogen specific dIgA antibody level determined from the assay to a reference response value/s to facilitate determining one or more of (i) the magnitude of the mucosal immune response to the pathogen or vaccine or (ii) the time since Start or start of infection, wherein the reference response value is obtained from the pre-determined level or change in the level of pathogen specific dIgA antibody over time from the biological sample in reference subjects. In one embodiment, the reference response level is the area under the curve (AUC) provided by plotting time over dIgA level or an indication thereof (eg axin units, weight/volume, label intensity or concentration).

In one embodiment, the method further comprises measuring or determining the level of pathogen specific total immunoglobulin or pathogen specific IgG in the sample.

In one embodiment, the antigen is the same for measuring dIgA and total Ig or IgG.

In one embodiment, the level or change in the level of dIgA antibody in the circulation of a subject provides an estimate of the level or change in the level of pathogen specific SIgA antibodies at a mucosal surface in the subject. Similarly, where the total or total pathogen specific dIgA response is estimated from the AUC or similar measure, this provides a further indication of the magnitude of the mucosal immune response to the pathogen. This also provides an indication of the level of neutralizing antibodies, which could be confirmed with a separate neutralization assay, if required.

In one embodiment, the method comprises measuring the pathogen neutralizing ability of immunoglobulin or dIgA in the sample.

In one embodiment, the method comprises contacting a biological sample comprising antibodies such as blood, serum or plasma, or a fraction thereof, from a subject with a dIgA binding agent to allow formation of a complex between pathogen specific dIgA and the dIgA binding agent and determining the level of pathogen specific dIgA therefrom.

In one embodiment, the dIgA binding agent is binding agent is selected from: an anti-dIgA antibody, an anti-J-chain antibody, pIgR or a modified pIgR that binds dIgA and substantially fails to bind IgM, a dIgA receptor or soluble ligand. In one embodiment, the pIgR is a chimeric human pIgR comprising a non-human domain 1 that substantially fails to bind IgM.

In one embodiment, the measured level of dIgA is assessed by visual comparison or instrument reader and/or associated software adapted to evaluate antibody levels and compare data. In one embodiment, the method is suitable for use in a point-of-care (POC) or near POC device.

In one embodiment, the method comprises: ECLIA, IFA, ELISA-type, flow cytometry (e.g., fluorescent microbeads, nanoparticles), bead array, lateral flow, interferometry, cartridge, microfluidic, förster resonance energy transfer, immunochromatographic based methods or formats or the like.

In one embodiment, the method is performed using a lateral flow or microfluidic device.

In one embodiment, at least step (i) is performed on a lateral flow device comprising a test strip comprising at least one sample loading region, wherein
a) the strip comprises a capture portion comprising an agent which specifically binds dIgA.

In one embodiment, pathogen specific dIgA bound to a dIgA binding reagent is detected using a labelled or tagged pathogen or antigen, such as colloidal gold labelled pathogen or antigen.

dIgA Binding Agents

As used herein, the term “binds” or “binding” includes the interaction of a binding agent e.g. a protein or antibody or an antigen binding domain thereof with an antigen and that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labelled “A” and the antibody, will reduce the amount of labelled “A” bound to the antibody. Antibodies also bind other components at the mucosal epithelial, such as mucins that form a major part of epithelial mechanisms of defence against invading pathogens.

As used herein, the term “specifically binds” or “specific for X” shall be taken to mean a binding agent of the disclosure reacts or associates more frequently, more rapidly, with greater duration and/or with greater affinity with a particular antigen or antigens or cell expressing same than it does with alternative antigens or cells. For example, a protein that specifically binds to an antigen binds that antigen with greater affinity (e.g., 20 fold or 40 fold or 60 fold or 80 fold to 100 fold or 150 fold or 200 fold greater affinity), avidity, more readily, and/or with greater duration than it binds to other antigens, e.g., to other subclasses of IgA or to antigens commonly recognized by polyreactive natural antibodies (i.e., by naturally occurring antibodies known to bind a variety of antigens naturally found in humans). It is also understood by reading this definition that, for example, a protein that specifically binds to a first antigen may or may not specifically bind to a second antigen.

In one embodiment, the method comprises contacting the sample with a dIgA binding agent and forming a detectable dIgA complex. In one embodiment, the method comprises contacting the sample with a pIgR or anti-IgA J chain antibody and forming a detectable dIgA complex. In one embodiment, the detectable complexes referred to herein are directly detectable or indirectly detectable. Reference to indirect detections, will as known in the art includes the use of a further interacting molecule to effect detection. Detection itself may be visually detected or by instrument.

In one embodiment, the detectable complexes described herein are directly detectable. In one embodiment, the binding agent is conjugated to a detectable label or marker or microparticles comprising a detectable marker that provide a detectable signal.

The agent specifically binding dIgA in the sample can be any binding agent that binds to dIgA under the required conditions and optionally forms a detectable complex. The binding agent may conveniently be an antibody or an antigen-binding fragment thereof. Other suitable binding agents are known in the art and include antigen binding constructs such as binding proteins, binding peptides, affimers, affibodies, aptamers, nanobodies and mimetics.

Methods of making antigen-specific binding agents, including antibodies and their derivatives and analogs and mimetics thereof, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, New Jersey and the pSKAN Phagemid Display System, commercially available from MoBiTec, LLC of Marco Island, Florida. Aptamer technology is described for example and without limitation in U.S. Pat. Nos. 5,270,163; 5,475,096; 5,840,867 and 6,544,776. The skilled person will be able to select binding agents for use in the methods as described herein.

In one embodiment, the dIgA binding agent is an antibody or an antigen-binding fragment or derivative thereof, an antigen-binding construct such as an affimer, affibody, aptamer, nanobody and mimetics or a ligand or binding part thereof. In one embodiment, the binding agent is immobilised on a support, such as, lateral flow test strip. In some embodiment the agent is immobilised on a solid support, affinity matrix, bead etc for identification and/or purification.

In an embodiment, the binding agent is a pIgR or pIgR based binding agent. The polymeric immunoglobulin receptor (pIgR) is encoded by the PIGR gene and is expressed in mucosal epithelial cells where it facilitates uptake of dIgA and secretion of SIgA. pIgR has five immunoglobulin-like domains which bind to dIgA including to the J-chain thereof. Human pIgR also binds to pentameric IgM while rabbit pIgR does not bind IgM or only very weakly.

The term “pIgR” as used herein refers to the polymeric Ig receptor including recombinant and modified forms thereof that specifically bind dIgA. In some embodiments, the pIgR is a pIgR as described in the Applicants previous application WO2014/071456. In some embodiments, the pIgR is produced in glycan deficient cells such as glycan deficient CHO cells to enhance preferential binding to dIgA over IgM. In one embodiment, pIgR is modified to bind dIgA but not substantially bind IgM by removal of human domain 1. In an embodiment, human domain 1 is replaced by rabbit domain 1. In some embodiments, the recombinant pIgR is derived from a primate such as human pIgR and comprises at least one immunoglobulin-like domain derived from a non-primate such as rabbit, mouse, or rat. In some embodiments, the recombinant pIgR comprises an amino acid sequence set out in SEQ ID NO: 2, or SEQ ID NO: 4, or SEQ ID NO: 6, or SEQ ID NO: 12, or SEQ ID NO: 14, or SEQ ID NO: 16 set out in WO/2014/071456 or an dIgA-binding part thereof or and a dIgA binding variant thereof. In an embodiment, the pIgR is the chimeric secretory component. The chimeric secretory component comprises rabbit domain 1 and human domains 2-5 as described in SEQ ID NO: 5 or SEQ ID NO: 6 of WO/2014/071456. In an embodiment, pIgR is conjugated to a detectable tag or moiety. In some embodiments, the recombinant pIgR lacks a transmembrane domain (ATM). In other embodiments, the recombinant pIgR lacks a cytoplasmic domain. In some embodiments, the recombinant pIgR lacks a TM domain and a cytoplasmic domain (ACYT). In some embodiments, recombinant pIgR comprises a substitution in the cytoplasmic domain and provides a heterologous cytoplasmic domain. Various forms of recombinant pIgR are contemplated. In other embodiments, the recombinant pIgR is bound to a solid support. Solid supports include plates, wells, beads, agarose particles, nitrocellulose strips, etc. In some embodiments, recombinant pIgR is produced in glycan deficient cells such as glycan deficient CHO cells. In some embodiments, the recombinant pIgR is derived from a primate such as human pIgR and comprises at least one immunoglobulin-like domain derived from a non-primate such as rabbit. Methods of making CSC/dIgA binding pIgRs are provided in Roe M, Norderhaug I N, Brandtzaeg P, Johansen F E. Fine specificity of ligand-binding domain 1 in the polymeric Ig receptor: importance of the CDR2-containing region for IgM interaction. J Immunol. 1999; 162:6046-52. See also Braathen R, Sorensen V, Brandtzaeg P, Sandlie I, Johansen F E. The carboxyl-terminal domains of IgA and IgM direct isotype-specific polym-erization and interaction with the polymeric immunoglobulin receptor. J Biol Chem. 2002; 277:42755-62.

In one embodiment, the level or presence of dIgA is measured using a polymeric Ig Receptor (pIgR) that binds dIgA and substantially fails to bind other antibody isotypes (monomeric IgA, IgM, IgG, IgE, IgD). In one embodiment, the pIgR is a chimeric form of human pIgR comprising domain 1 of another species whose pIgR does not bind IgM. In one embodiment the species is rabbit. In another embodiment, the pIgR is derived from a non-human animal whose pIgR does not bind IgM (eg, rabbit).

Variants are functional and include deletion, substitution and insertional variants. Illustrated herein are human derived pIgR varied by one or more immunoglobulin domains (D). Variants include “parts” which includes fragments comprising from about 50%, 60%, 70%, 80%, 85%, 90%, 95% of the reference sequence. Substitution for an equivalent domain from a lower mammal such as a rat, mouse or rabbit domain. “Variants” of the recited amino acid sequences are also contemplated. Variant molecules are designed to retain the dIgA binding functional activity of the pre-modified recombinant pIgR or to exhibit enhanced activity. Polypeptide variants according to the invention can be identified either rationally, or via established methods of mutagenesis (see, for example, Watson, J. D. et al., “Molecular Biology of the Gene”, Fourth Edition, Benjamin/Cummings, Menlo Park, California, 1987). Random or site-directed mutagenesis can be employed. Illustrative amino acids affect glycosylation of the recombinant pIgR. Polypeptides, resulting from rational or established methods of mutagenesis or from combinatorial chemistries, may comprise conservative amino acid substitutions. It is well understood in the art that some amino acids may be changed to others with broadly similar properties without changing the nature of the activity of the polypeptide. Such conservative substitutions are understood and contemplated.

Variant pIgR polypeptides comprises at least 50% sequence identity to a herein provided amino acid sequence at least over the immunoglobulin-like domain region. The use of recombinant pIgR is also highly advantageous not least because the reagent displays low background (at least 50% less background compared to antibody based reagents) in binding assays unlike most antibody based binding agents. In addition, recombinant pIgR displays high thermal stability. For example, lyophilised recombinant pIgR retained 50% activity at 60° C. and 100% activity at 45° C. after three weeks prior to reconstitution, which compares favourably to the rapid loss of activity for dried anti-IgM antibody under the same conditions.

Suitable binding agents for determining the dIgA level include any binding agent that specifically or non-specifically binds dIgA and forms a detectable dIgA complex. In an embodiment, the binding agent is non-specific binding other forms or IgA which may be removed from the sample by binding with another binding agent before use with the dIgA binding agent. One example is human pIgR which binds dIgA and IgM. In one embodiment, the binding agent is specific for dIgA. In an embodiment, the binding agent is a dIgA antibody. In one embodiment, the binding agent is an anti-dIgA1 antibody, an anti-dIgA2 antibody or a combination thereof. In one embodiment, the binding agent is an antibody which binds the J-chain of dIgA (e.g. anti-IgA J chain LifeSpan Biosciences Cat no. LS-B12942 or OriGene Technologies Cat no. AM20272PU-M).

In one embodiment, pIgR such as human pIgR or the J-chain is used in nucleic acid or protein form to combine with dIgA to produce SIgA for use in pharmaceutical compositions. Illustrative compositions include formulations for mucosal surface delivery such as spray, aerosol, nebuliser, topical, intra nasal, sub lingual etc.

In one embodiment, pIgR, such as CSC, is bound to a solid or semi solid support such as an affinity chromatography support/matrix, agarose beads, to facilitate dIgA purification for use as a therapeutic or prophylactic composition for use in therapy, for use in treating or preventing infection by an infections pathogen, such as CoV or a pathogenic antigen such as venom where an effective and early mucosal neutralizing immune response is important.

In one embodiment, a pIgR such as CSC is used to remove, purify, deplete, or capture at least 20% to 50%, at least 55%, at least 60%, at least 65%, at least 70%, at least 75%, at least 80%, at least 85%, at least 90%, at least 95%, at least 98%, at least 99%, or 100% of the dIgA, or pathogen specific dIgA antibodies from a blood product obtained from a subject. In certain embodiments, the method removes, depletes, or captures at least 80%, at least 85%, at least 90%, at least 95%, at least 98%, at least 99%, or 100% of the dIgA or specific pathogen binding dIgA antibodies from a blood product obtained from a subject. Non-limiting examples of a blood product include whole blood, serum, plasma, the like, and a combination thereof. A blood product may be devoid of cells, or may include cells (e.g., red blood cells, platelets and/or lymphocytes). In one embodiment, the pathogen is CoV and antibodies are selected based on their binding specificity and/or affinity to CoV antigens such as Spike protei or a part thereof.

In one embodiment, a pIgR such as CSC is used to identifying and/or capture B-cell clones that express dIgA (rather than mIgA) (i.e. they express IgA as well as J-chain and therefore produce dIgA rather than mIgA). In one embodiment, native dIgA molecules expressed therefrom are cloned and expressed. Such dIgA molecules are formulated into pharmaceutical compositions and administered so as to provide therapeutic or prophylactic anti-pathogen or anti-pathogenic antigen effects.

It is proposed, without limitation, that such native IgA molecules that have undergone somatic maturation and expansion in the context of co-expression with J-chain (i.e., dIgA obtained from dIgA-positive B cells) will be more biologically active/relevant than native or recombinant mIgAs, such as obtained from dIgA-negative B cells (including IgG- or IgM-positive B cells cloned and expressed as IgA isotype), which are cloned and expressed as dIgA together with J chain.

In another embodiment, human B-cells from a pathogen infected person are captured or selected based on their pathogen/antigen binding profile, B-cells are immortalized, and cloned B-cells are used to isolate dIgA and IgG and IgM monoclonals. Monoclonal antibodies displaying neutralizing activity against the pathogen are selected. Antibody genes from IgG and IgM monoclonals are converted to IgA and expressed with J-chain to give dIgA, antibody genes from mIgA monoclonals are expressed with J-chain to give dIgA, while dIgA monoclonals may not need further modification (i.e. the monoclonal-producing hybridoma already expresses the desired dIgA without the need for recombinant cloning and expression). dIgA is administered and shows neutralizing activity in the mucosal niche. As shown in the Examples, dIgA forms of IgG monoclonal antibodies display as good

In one embodiment, the present application provides an anti-infectious pathogen immunoglobulin (AIdIgA) composition comprising: (a) a plurality of human antibodies comprising one or more antibodies that specifically bind to the infectious pathogen; and (b) a pharmaceutically acceptable diluent or carrier; wherein the one or more antibodies were isolated from a donor that has (ii) cleared the pathogen and exhibits an immune response comprising a level of dIgA antibodies against the pathogen or (ii) has generated an immune response to a vaccine against the pathogen, and exhibits a level of dIgA antibodies against the pathogen, as determined by blood sample testing.

In one embodiment, the plurality of human antibodies or composition comprising same, comprises a high level of dIgA relative to IgM or non-dIgA subtypes, at least about 70% to 90% dIgA subtype antibodies and not more than about 10% to 30% IgM or non-dIgA subtype antibodies.

In one embodiment, the composition comprises: (a) plasma proteins with at least about 80% dIgA, or at least 90% or at least about 95% dIgA, with not more than about 5% non-dIgA-contaminating proteins; and/or (b) at least about 96%, about 97%, about 98%, about 99%, or about 100% dIgA. In one embodiment, the dIgA is pathogen or pathogenic antigen specific. In one embodiment the dIgA is CoV specific. In one embodiment, the dIgA is Spike specific. In one embodiment, the dIgA is venom specific.

In one embodiment, (a) the plurality of antibodies is prepared from donor blood, serum, or plasma; and/or (b) the plurality of antibodies comprises homologous immunoglobulins.

In one embodiment, the composition comprises (a) monoclonal antibodies produced recombinantly based on the sequences of polyclonal antibodies isolated from blood, serum or plasma from a convalescent donor or a donor that was immunized with a vaccine; and/or (b) polyclonal antibodies isolated from blood, serum or plasma from a convalescent donor or a donor that was immunized with a vaccine against the pathogen.

In one embodiment, the composition comprises (a) monoclonal antibodies produced recombinantly based on the sequences of polyclonal antibodies isolated from blood, serum or plasma from a hyperimmunised non-human animal donor; and/or (b) polyclonal antibodies isolated from blood, serum or plasma from a hyperimmunised non-human animal donor.

In one embodiment antibodies are selected that are neutralizing and dIgA.

In one embodiment, the present specification described and enables a method of preparing a pharmaceutical composition comprising anti-pathogen dIgA immunoglobulin as the active agent for treatment or prophylaxis of an infection by the pathogen, the method comprising: (a) identifying a donor as described herein (b) isolating from the donor a plurality of antibodies that bind a dIgA binding agent; (c) formulating the plurality of antibodies into a pharmaceutical composition.

In one embodiment, the dIgA binding agent is pIgR or a derivative thereof that binds dIgA and substantially does not bind IgM.

In one embodiment, the pathogen is SARS-COV-2.

In one embodiment, the AIdIgA is administered intravenously. In other embodiment, the agent is delivered orally or parenterally. In an embodiment, AIdIgA is administered sub-cutaneously. For the subcutaneous administration larger volumes may be needed and formulations used that are suitable for passage of the extracellular matrix of the subcutaneous tissue. In one embodiment, antibodies are co-formulated with agents such as recombinant human hyaluronidase to facilitate administration of larger volumes without pain. Other formulations provide for slow release into the circulation and subsequent clearance/excretion over a longer time period.

In one embodiment, the dIgA antibody or dIgA antigen binding derivative thereof binds to a pathogenic antigen at an EC50 of between about 1 ng/ml and about 100 ng/ml, 1 ng/ml and about 50 ng/ml, or between about 1 ng/ml and about 25 ng/ml, or less than about 50 ng/ml or 25 ng/ml; or between about 1 ng/ml and about 500 ng/ml, or between about 1 ng/ml and about 250 ng/ml, or between about 1 ng/ml and about 50 ng/ml, or less than about 500 ng/ml, 250 ng/ml or 100 ng/ml.

In one embodiment, a method of making an antibody is provided by identifying a circulating antibody with activity from a subject comprising subjecting a blood product to one or more rounds of affinity chromatography using a dIgA binding agent to purify the circulating dIgA antibody; ii) optionally further subjecting the circulating antibody to isoelectric focusing to purify the circulating antibody based on charge or size exclusion chromatography. In one embodiment the purified circulating dIgA antibody is tested for neutralizing or other functional activity. In one embodiment, the method comprises digesting the purified circulating antibody to create an antibody fragment and subjecting the antibody fragment to mass spectrometry to generate a mass assignment and a deduced amino acid sequence of the antibody fragment. The deduced amino acid sequence is compared with an amino acid sequence of an antibody generated from the subject's B-cells to identify an antibody sequence that matches the deduced amino acid sequence. Then the skilled person generates a dIgA antibody comprising light chain and heavy chain CDR sequences of the B-cell antibody that matches the deduced amino acid sequence, and tests again for activity.

In another embodiment, the native (HSC) form of pIgR could be used for selecting dIgA and remain linked to the dIgA, making a synthetic version of SIgA which would have a longer plasma half-life (since no active transport mechanism to remove it) but would lack targeting to the mucosal surfaces. As human pIgR also binds IgM this would also need to be removed to make a predominantly SIgA product if the starting material was human serum or plasma, but this would be preferable to the use of CSC which could provoke an immune response to the non-human amino acid sequences in D1.

A “solid substrate” refers to, for example, a material having a rigid or semi-rigid surface or surfaces, which may be regular or irregular, and may take the form of beads, resins, gels, spheres, microspheres, particles, fibres or other geometric configurations or physical forms. A solid substrate typically comprises a material that is applicable in medical, biochemical or biological assays, for example, substrate used in apheresis, column chromatography for purification or separation of biological molecules or organic molecules and ELISA assays. Solid substrates may be porous or non-porous.

Solid substrates or surfaces for immobilizing the affinity matrix that binds to dIgA antibodies are known in the art. Non-limiting examples of solid substrates include for example, polymers such as polysaccharides, in particular polysaccharides having a molecular weight of 100 kDa or more, such as agarose. Agarose may be in particulate form, which optionally can be cross-linked. A particular example of agarose is Sepharose, or cellulose, which can be cross-linked. Other polymers appropriate as a substrate include, for example, carboxylated polystyrene. Solid substrates may be provided in the form of magnetic beads. Glass is also an appropriate substrate material. Any suitable blood or plasma filtration column or system can be adapted for the present process. Non-limiting examples include columns described in U.S. Pat. No. 4,619,639, membrane filtration systems (e.g., MDF) used with suitable particles, surfaces, or substrates, and PlasmaFlo® OP-05(W)L and RheoFilter® AR2000 blood filters manufactured by Asahi Medical Company, Ltd. of Japan.

Other binding reagents may be employed in the present assays to detect other immunoglobulins or as controls. In one embodiment, the binding agent is Protein L. The term “Protein L” refers to an immunoglobulin (Ig) binding protein original derived from the bacteria Peptostreptococcus magnus. Protein L can bind the kappa light chain of antibodies without interfering with the antibodies antigen binding site. It can bind all classes of Ig (IgG, IgM, IgA, IgE and IgD). The term is intended to cover modified and recombinant versions of Protein L. Protein L can be obtained, for example, from ThermoFisher Scientific (Protein L Cat no. 21189).

### Assay Formats

The method wherein the measured level of dIgA is assessed by visual comparison or instrument reader and/or associated software adapted to evaluate antibody levels and compare data.

In one embodiment, the method comprises: ECLIA, IFA, ELISA-type, flow cytometry (e.g., fluorescent microbeads, nanoparticles), bead array, lateral flow, interferometry methods, cartridge, microfluidic, förster resonance energy transfer, immunochromatographic based methods or formats or the like, nucleic acid based e.g., crisper based methods.

The method is suitable for in vitro use and may be performed in an assay format known to a person skilled in the art, including as an immunoassay, chromatographic assay or a homogenous assay.

As used herein, “immunoassay” refers to assays using specific binding agents such as immunoglobulins or parts thereof that are capable of detecting and quantifying a desired biomarker such as dIgA. The immunoassay may be one of a range of immune assay formats known to the skilled addressee. A wide range of immunoassay techniques are available, such as those described in Wild D. “The Immunoassay Handbook” Nature Publishing Group, 4th Edition, 2013 and subsequent innovations.

Electrochemiluminescence (ELICA), enzyme-linked immunosorbet assay (ELISA), fluorescent immunosorbent assay (FIA) and Luminex LabMAP immunoassays are examples of suitable assays to detect levels of the biomarkers. In one example, a binding agent e.g. an antibody or Protein L is attached to a support surface and a further binding reagent/antibody comprising a detectable group binds to the antibody or a substrate bound by the antibody. Examples of detectable-groups include, for example and without limitation: fluorochromes, enzymes, epitopes for binding a second binding reagent (for example, when the second binding reagent/antibody is a mouse antibody, which is detected by a fluorescently-labelled anti-mouse antibody), for example an antigen or a member of a binding pair, such as biotin. The surface may be a planar surface, such as in the case of a typical grid-type array (for example, but without limitation, 96-well plates and planar microarrays) or a non-planar surface, as with coated bead array technologies, where each “species” of bead is labelled with, for example, a fluorochrome (such as the Luminex technology described in U.S. Pat. Nos. 6,599,331, 6,592,822 and 6,268,222), or quantum dot technology (for example, as described in U.S. Pat. No. 6,306,610). Such assays may also be regarded as laboratory information management systems (LIMS):

Lateral flow assays and more recently non-lateral flow and microfluidics provide a useful set up for biological assays. Such assays can be qualitative, quantitative or semi quantitative. In microfluidic devices, small volumes of liquid are moved through microchannels generated in, for example, a chip or cartridge. A wide range of detection reagents are available including metal nanoparticles, coloured or luminescent materials. Resonance enhanced adsorption (REA) of bioconjugated metal nanoparticles offers rapid processing times and other advantages. These devices have been combined with barcode technologies to identify the patient and the analyte being tested. Computer software and hardware for assessing input data are encompassed by the present disclosure. Point-of-care devices and arrays and high throughput screening methods are also contemplated.

Qualitative assays providing an intermediate or definitive diagnosis require integrated thresholds, gates or windows that permit scoring of samples as likely or not to have a condition. Instrument readers and software are often employed to collate data and process it through a diagnostic algorithm or decision tree

In the bead-type immunoassays, the Luminex LabMAP system can be utilized. The LabMAP system incorporates polystyrene microspheres that are dyed internally with two spectrally distinct fluorochromes. Using precise ratios of these fluorochromes, an array is created consisting of different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

In one embodiment, the assay is a homogenous assay, meaning an assay format allowing the make an assay-measurement by a simple mix and read procedure without the necessity to process samples by separating or washing. Such assays do not include an immunosorbent solid phase step. In one embodiment, the homogenous assay is time-resolved Förster resonance energy transfer (FRET).

In one embodiment, the assay is a flow cytometry, bead array, lateral flow, cartridge, microfluidic or immunochromatographic based method or the like. In one embodiment, the assay is a point-of-care assay. In one embodiment, the point-of-care assay reader is an Axxin AX-2X-type reader, or equivalent or modified device. For example, the device may be modified to include LEDs and filters of the appropriate wavelength for the subject assays.

As used herein, the term “binds specifically,” and the like when referring to an antigen-binding molecule refers to a binding reaction which is determinative of the presence of an antigen in the presence of a heterogeneous population of proteins and other biologics. Thus, under designated immunoassay conditions, the specified antigen-binding molecules bind to a particular antigen and do not bind in a significant amount to other proteins or antigens present in the sample. Specific binding to an antigen under such conditions may require an antigen-binding molecule that is selected for its specificity for a particular antigen. For example, antigen-binding molecules can be raised to a selected protein antigen, which bind to that antigen but not to other proteins present in a sample. A variety of immunoassay formats may be used to select antigen-binding molecules specifically immuno-interactive with a particular protein. For example, solid-phase ELISA immunoassays are routinely used to select monoclonal antibodies specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity.

For example, specific recognition is provided by a primary antibody (polyclonal or monoclonal) and a secondary detection system is used to detect presence (or binding) of the primary antibody. Detectable labels can be conjugated to the secondary antibody, such as a fluorescent label, a radiolabel, or an enzyme (e.g., alkaline phosphatase, horseradish peroxidase) which produces a quantifiable, e.g., colored, product. In another suitable method, the primary antibody itself can be detectably labeled. For example, a protein-specific monoclonal antibody, can be used both as an immunoadsorbent and as an enzyme-labelled probe or other labelled probe, to detect and quantify complexes formed in the present process or kit.

The amount of such protein present in a sample can be calculated by reference to the amount present in a standard or reference preparation using a linear regression computer algorithm (see Iacobilli et al., (1988) Breast Cancer Research and Treatment 11:19-30). In other embodiments, two different monoclonal antibodies to the protein of interest can be employed, one as the immunoadsorbent and the other as an enzyme-labelled probe.

Additionally, recent developments in the field of protein capture arrays permit the simultaneous detection and/or quantification of a large number of proteins. For example, low-density protein arrays on filter membranes, such as the universal protein array system (Ge (2000) Nucleic Acids Res. 28(2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-vaccine administration. Protein capture arrays typically comprise a plurality of protein-capture agents each of which defines a spatially distinct feature of the array. The protein-capture agent can be any molecule or complex of molecules which has the ability to bind a protein and immobilize it to the site of the protein-capture agent on the array. The protein-capture agent may be a protein whose natural function in a cell is to specifically bind another protein, such as an antibody or a receptor. Alternatively, the protein-capture agent may instead be a partially or wholly synthetic or recombinant protein which specifically binds a protein. Alternatively, the protein-capture agent may be a protein which has been selected in vitro from a mutagenized, randomized, or completely random and synthetic library by its binding affinity to a specific protein or peptide target. The selection method used may optionally have been a display method such as ribosome display or phage display, as known in the art. Alternatively, the protein-capture agent obtained via in vitro selection may be a DNA or RNA aptamer which specifically binds a protein target (see, e.g., Potyrailo et al., (1998) Anal. Chem. 70:3419-3425; Cohen et al. (1998) Proc. Natl. Acad. Sci. USA 95:14272-14277; Fukuda, et al. (1997) Nucleic Acids Symp. Ser. 37:237-238; available from SomaLogic). For example, aptamers are selected from libraries of oligonucleotides by the Selex™ process and their interaction with protein can be enhanced by covalent attachment, through incorporation of brominated deoxyuridine and UV-activated crosslinking (photoaptamers). Aptamers have the advantages of ease of production by automated oligonucleotide synthesis and the stability and robustness of DNA; universal fluorescent protein stains can be used to detect binding. Alternatively, the in vitro selected protein-capture agent may be a polypeptide (e.g., an antigen) (see, e.g., Roberts and Szostak (1997) Proc. Natl. Acad. Sci. USA 94:12297-12302).

An alternative to an array of capture molecules is one made through ‘molecular imprinting’ technology, in which peptides (e.g., from the C-terminal regions of proteins) are used as templates to generate structurally complementary, sequence-specific cavities in a polymerisable matrix; the cavities can then specifically capture (denatured) proteins which have the appropriate primary amino acid sequence (e.g., available from ProteinPrint™ and Aspira Biosystems).

Exemplary protein capture arrays include arrays comprising spatially addressed pathogen antigens or antibody binding agents, which can facilitate extensive parallel analysis of numerous antigens and antibodies. Such arrays have been shown to have the required properties of specificity and acceptable background, and some are available commercially (e.g., BD Biosciences, Clontech, BioRad and Sigma). Various methods for the preparation of arrays have been reported (see, e.g., Lopez et al. (2003) J. Chromatogr. B 787: 19-27; Cahill (2000) Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210).

Immunoglobulin antigen-binding molecules are made either by conventional immunization (e.g., polyclonal sera and hybridomas), or as recombinant derivatives or fragments, usually expressed in E. coli, after selection from phage display or ribosome display libraries (e.g., available from Cambridge Antibody Technology, BioInvent, Affitech and Biosite). Many different antibody expression systems are now available. For example, plant cells are able to reproduce the complexity of human proteins. The homologies shared by the protein biosynthesis and maturation machinery in animal and plant cells are well illustrated through the ability of plants to produce various types of recombinant antibodies, such as IgGs or secretory IgAs (SIgAs).

Alternatively, ‘combibodies’ comprising non-covalent associations of VH and VL domains, can be produced in a matrix format created from combinations of diabody-producing bacterial clones (e.g., available from Domantis). Exemplary antigen-binding molecules for use as protein-capture agents include monoclonal antibodies, polyclonal antibodies, Fv, Fab, Fab′ and F(ab′)₂ immunoglobulin fragments, synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv, single domains from camelids or engineered human equivalents.

Individual spatially distinct protein-capture agents are typically attached to a support surface, which is generally planar or contoured. Common physical supports include glass slides, silicon, microwells, nitrocellulose or PVDF membranes, and magnetic and other microbeads.

While microdrops of protein delivered onto planar surfaces are widely used, related alternative architectures include CD centrifugation devices based on developments in microfluidics (e.g., available from Gyros) and specialized chip designs, such as engineered microchannels in a plate (e.g., The Living Chip™, available from Biotrove) and tiny 3D posts on a silicon surface (e.g., available from Zyomyx).

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second or subsequent protein in the sample is already known or known to be invariant.

In an illustrative example, fluorescence labeling can be used for detecting protein bound to the array. The same instrumentation as used for reading DNA microarrays is applicable to protein-capture arrays. For differential display, capture arrays (e.g. antibody arrays) can be probed with fluorescently labeled proteins from or are labeled with different fluorophores (e.g., Cy-3 and Cy-5) and mixed, such that the color acts as a readout for changes in target abundance. Fluorescent readout sensitivity can be amplified 10-100 fold by tyramide signal amplification (TSA) (e.g., available from PerkinElmer Lifesciences). Planar waveguide technology (e.g., available from Zeptosens) enables ultrasensitive fluorescence detection, with the additional advantage of no washing procedures. High sensitivity can also be achieved with suspension beads and particles, using phycoerythrin as label (e.g., available from Luminex) or the properties of semiconductor nanocrystals (e.g., available from Quantum Dot). Fluorescence resonance energy transfer has been adapted to detect binding of unlabelled ligands, which may be useful on arrays (e.g., available from Affibody). Several alternative readouts have been developed, including adaptations of surface plasmon resonance (e.g., available from HTS Biosystems and Intrinsic Bioprobes), rolling circle DNA amplification (e.g., available from Molecular Staging), mass spectrometry (e.g., available from Sense Proteomic, Ciphergen, Intrinsic and Bioprobes), resonance light scattering (e.g., available from Genicon Sciences) and atomic force microscopy (e.g., available from BioForce Laboratories). A microfluidics system for automated sample incubation with arrays on glass slides and washing has been co-developed by NextGen and Perkin Elmer Life Sciences.

In certain embodiments, the techniques used for detection of dIgA or other preselected products will include internal or external standards to permit quantitative or semi-quantitative determination of those products, to thereby enable a valid comparison of the level or functional activity of these expression products in a biological sample with the corresponding expression products in a reference sample or samples. Such standards can be determined by the skilled practitioner using standard protocols. In specific examples, absolute values for the level or functional activity of individual expression products are determined.

In one embodiment, methods for detecting antigen specific dIgA in a sample such as human serum or plasma are provided. For example, CSC is immobilised on the ELISA plate, and incubated with serum or other samples. Dimeric IgA is captured on the solid phase, and after optionally washing to remove other sample components (such as IgA and IgG that are not captured), the presence of antigen-specific dIgA is detected by sequential addition of antigen that is, for example, either biotinylated, or reacted with a biotinylated monoclonal antibody against the antigen, and streptavidin-HRP. In this way, any antigen that is immobilised by reaction with antigen-specific dIgA will give a signal through the biotin-streptavidin interaction or an equivalent reagent.

Alternatively, for example, antigen is coated directly onto the ELISA surface. Biological samples comprising antibodies are applied to the plate and antigen-specific antibodies, including IgM and dIgA, bind to the antigens and are then dIgA detected with CSC, or other dIgA specific binding agent. There are manifold methods of detecting or measuring bound moieties. In one embodiment, a signal may be generated with TMB substrate or equivalent reagent.

In various related aspects, the present invention also relates to devices and kits for performing the methods described herein. Suitable kits comprise at least some, preferably all of, the reagents sufficient for performing at least one of the methods described herein.

In an aspect, the present invention provides a kit for assessing the immune response to a pathogen or vaccine in a subject. In one embodiment, the level of pathogen specific dIgA is calibrated against a time since infection/vaccination in order to establish the magnitude of the mucosal immune response.

In an aspect, the present application enables kits for assessing the time since infection by a mucosal pathogen. This is possible, again, because of the reliable trajectory of dIgA levels in the blood after infection and before they fall below a threshold or become undetectable at about 2 to 3 month later.

In one embodiment, the kit comprises CSC or other pIgR-based dIgA binding molecule.

In one embodiment, the kit comprises a strip, chip or cartridge for use in a lateral flow assay. In one embodiment, the kit comprises a strip, chip or cartridge for use on point-of care device.

In one embodiment, the kit comprises a test strip for a lateral flow device comprising at least one sample loading region, wherein
a) the strip comprises a capture portion comprising an agent which binds dIgA.

A sample can flow through the device, simultaneously or subsequently a detection reagent is flowed through the device which binds dIgA. Detecting the detection reagent bound to dIgA can be performed in a subsequent step e.g. by measuring absorbance. In an embodiment, the detection reagent comprises colloidal gold.

In one embodiment, the kit can be stored at about 4° C. In one embodiment, the kit can be stored at about 15° C. In one embodiment, the kit can be stored at about 23° C. In one embodiment, the kit can withstand exposure to temperatures of 37° C. or 45° C. for at least one week for example during shipping.

In one embodiment, the kit comprises an immunoassay test strip. In one embodiment, the immunoassay test strip comprises a sample loading portions comprising binding agents and two or more capture portions. In one embodiment, the biological sample is contacted with the binding agents by applying the sample to a sample portion of an immunoassay test strip wherein the test strip sample portion is operably connected to spaced capture portions of the test strip and whereby the components of the sample flow from the test strip sample portion to and through the test strip capture portions, and wherein one capture portion comprises a binding agent for detecting dIgA, and wherein a second capture portion comprises a binding agent for detecting dIgA, and wherein at least one of the binding agents specifically detects their target. In one embodiment, the binding agent for detecting dIgA binds specifically with dIgA. In one embodiment, the sample contacts the capture portion comprising a binding agent for detecting low dIgA before contacting the capture portion comprising a binding agent for detecting high dIgA.

In one embodiment, one binding region (a) is closer to the sample loading region than another (b) such that the sample contacts a) before b). Results may be determined by reference to a visual standard which may be part of test strip or part of the kit. Or, by reference to an instrument reader. In one embodiment, the strip further comprises a capture portion comprising an agent specific for binding a control. In one embodiment, the sample is flowed through the device and simultaneously or subsequently with the flow of a detection reagent through the device, and then detecting the detection reagent bound to antibody or a complex comprising same. In one embodiment, the detection reagent comprises a labelled antigen or pathogen.

In one embodiment, the kit is for use in performing all of part of an assay herein disclosed. In one embodiment, the disclosure enables and provides a point-of-care device capable of performing the methods disclosed and claimed herein.

In one aspect, the method is performed using a lateral flow or microfluidic device wherein at least one measuring step is performed on a lateral flow device comprising a test strip comprising at least one sample loading region, wherein the strip or equivalent surface or medium comprises a capture portion comprising a binding (non-antibody) agent which binds dIgA.

In one aspect, the invention provides a kit or POC device comprising a test strip comprising at least one sample loading region, wherein
a) the strip comprises a capture portion comprising an agent which binds dIgA and directly or indirectly indicates+/−a first level of dIgA, and
b) the strip comprises a second capture portion comprising an agent which binds dIgA and directly or indirectly indicates+/−a second level of dIgA.

In one aspect, the invention provides a kit or POC device comprising a test strip comprising at least one sample loading region, wherein
a) the strip comprises a capture portion comprising an agent which binds dIgA and directly or indirectly indicates+/−a level of dIgA, and
b) the strip comprises a second capture portion comprising an agent which binds IgG and directly or indirectly indicates+/−a level of IgG in the sample applied to the device.

In one aspect, the invention provides a kit or POC device comprising a test strip comprising at least one sample loading region, wherein
a) the strip comprises a capture portion comprising an agent which binds dIgA and directly or indirectly indicates+/−a level of dIgA, and
b) the strip comprises a second capture portion comprising an agent which binds IgG and directly or indirectly indicates+/−a level of total Ig in the biological sample applied to the device.

In one embodiment, the dIgA is pathogen or antigen specific dIgA and the kit or device comprises at least in use, an antigen for discriminating between dIgA and antigen specific dIgA.

In one embodiment, non-antibody binding agent, such as an dIgA specific pIgR (eg. CSC) and anti-SC are co-combined with the patient sample (ie. simultaneously, concurrently, sequentially but before loading the test device with the patient sample. Here, for example the anti-SC binds to the CSC-dIgA to detect or measure the levels of circulating dIgA. As anti-SC will also detect SIgA in mucosal samples, it could be convenient to measure SIgA in mucosal samples at the same time or as an alternative to measuring dIgA in blood. Thus the device can be used to detect dIgA in plasma/serum or SIgA in other fluids. In one embodiment, the only difference would be the sample dilution with mucosal samples being uses in a more concentrated from such as a 1:10 dilution rather than a 1:100 (say) dilution for plasma.

In one embodiment, a procedural control is included in the kit or test strip/device. A high level of IgG measured contemporaneously or separately, may lead to re-classifying the plasma as not suitable for convalescent plasma therapy.

In some embodiments, the kit comprises:
a) an immunographic device comprising a porous membrane operably connected to a sample portion, a test portion, and optionally a control portion; and further comprising a sucker portion, portion comprising a dIgA binding agent, optionally a portion comprising an antigen and optionally a conjugate portion; and
b) instructions for using the immunographic device to detect the presence of antigen specific dIgA antibody in the sample.

### Thresholds—Generally

The person skilled in the art will appreciate that many devices can be programed and automated to detect differences from the thresholds as described herein.

Based on the disclosure herein a person skilled in the art will appreciate that the established level or levels of dIgA can be compared relative to a threshold (which may be population specific) to determine if a subject has a specific level such as a high level, a medium level or a low level of dIgA. In an embodiment, comparison to a threshold can be used to determine if a subject has mounted a functional mucosal immune response to a pathogen. In this embodiment, the time since infection or vaccination can be used as determined herein to quantify the response. Alternatively, whether a subject should be prioritised for convalescent plasma therapy or as a plasma donor for the preparation of a composition comprising dIgA as described herein.

The term “threshold” refers to a value or cut-off that must be met, exceeded or not exceeded to determine the next steps. The threshold can be set relative to a control or standard processed at the same time as the test. Alternatively, the threshold may be predetermined based on a data set produced using the specific reagents and platform for a given embodiment of the test.

In one embodiment, the threshold is a colour intensity that can be assessed visually or by instrument reader or simply by instrument. In one embodiment, the colour or fluorescence (for example) intensity can be represented as a numerical value or numerical range. As the skilled person will appreciate there are many different methods encompassed for detecting and quantifying an analyte which may be directly or indirectly labelled, tagged, bound or modified to provide a detectable signal.

In an embodiment, the threshold is set relative to a control. The term “control” includes any sample or group of samples that can be used to establish a knowledge base of data from a subject or subjects with a known disease status.

In one embodiment, the threshold is set as the mean value of the control group, the mean value plus one standard deviation of the control group, the mean value plus two standard deviations of the control group, the mean value plus three standard deviations of the control group, or a preselected level in the control group. In one embodiment, the threshold is set as the mean plus one standard deviation of a control group. In one embodiment, the threshold is set as the mean plus two standard deviations of a control group. In one embodiment, the threshold is set as the mean plus three standard deviations of a control group.

In some embodiments, different thresholds may be required for populations with different diseases/conditions and/or different stages of a disease/conditions. An internal standard reflective of the thresholds described herein may include as an internal control in the methods and kits as described herein.

In one embodiment, of the method described herein a difference from the threshold indicates useful levels of dIgA or useful levels of functional mucosal immune response to a vaccine or infection. In one embodiment, the difference is an elevation relative to a threshold. In an embodiment, the level is elevated, by at least 2%, or by at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100%, or at least 200%, or at least 300% compared the threshold. In one embodiment, the difference is a decrease relative to a threshold. In an embodiment, the level is decreased is by at least 2%, or at least 5%, or at least 10%, or at least 15%, or at least 20%, or at least 25%, or at least 30%, or at least 35%, or at least 40%, or at least 45%, or at least 50%, or at least 60%, or at least 70%, or at least 80%, or at least 90%, or at least 100% compared to the threshold.

In one embodiment, an absorbance unit provided by a device that measures absorbance such as an Axxin AX-2X reader is employed. Based on the information provided herein, a person skilled in the art could readily determine the equivalent threshold on equivalent and similar devices. In an alternate embodiment, the results of the assay could be converted to mg/ml, mass/volume, mass per mass etc.

### A Web-Based System

In some embodiments, the method comprises: (a) generating data using a method as described herein; (b) transforming the data into computer-readable form; and (c) operating a computer to execute an algorithm, wherein the algorithm determines closeness-of-fit between the computer-readable data and pre-determined data describing the observed mathematical relationship between the level of dIgA and the time since start (e.g., infection, symptoms, current infection) and/or the area under the curve indicating, the level of dIgA at a time since infection, the presence or magnitude of a mucosal immune response and, optionally if time since start is known, the potential magnitude of the mucosal immune response. In some embodiments, the algorithm comprises an artificial intelligence program, such as a fuzzy logic, cluster analysis or neural network. The subject methods may also be used in a personalized or a population medicine approach in the management of pathology platforms.

The present disclosure provides a computer program and hardware for assessment in a subject once off, over time or in response to vaccination, treatment or other affectors. Values are assigned to complex levels which are stored in a machine readable storage medium. A computer program product is one able to convert such values to code and store the code in a computer readable medium and optionally capable of assessing the relationship between the stored data and incoming data and optionally a knowledge database.

The present specification therefore provides a web-based system where data on levels of dIgA are provided by a client server to a central processor which analyses and compares to one or more controls control and optionally considers other information such as time since infection, symptom onset, treatment or vaccination and provides a report useful for various application as described herein, including contact tracing, immune surveillance, work place screening, convalescent plasma assessments, vaccine efficacy tests, and other applications which stem from the present invention.

The assay may, therefore, be in the form of a kit or computer-based system which comprises the reagents necessary to form and detect the herein described antibody complexes and the computer hardware and/or software including an algorithm to facilitate determination and transmission of reports to a clinician.

### Neutralizing Activity

“Neutralization assays” include pseudo or surrogate neutralization assays assays or neutralizing assays employing replication competent virus that provide an indication of the ability of an agent to prevent infection or pathogenesis by a pathogen in a subject. For viral infectious agents this typically involves determining the ability of the agent to prevent infection of host cells.

Neutralization assays are useful for determining the ability of antibodies to act therapeutically or prophylactically in a subject against a given infectious agent. Conventionally, neutralizing antibodies for virus infections are measured by plaque reduction neutralization test (PRNT).

One high throughput method is described in Muruato et al Nature Communications 11: Article number 4059(2020) published 13 Aug. 2020. Here a reporter SARS COV-2 expressing a fluorescent signal is exposed to sera and the ability of the sera to prevent intracellular fluorescence is measured.

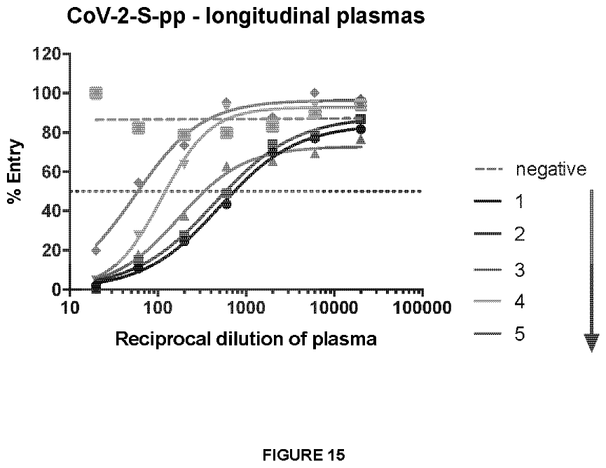

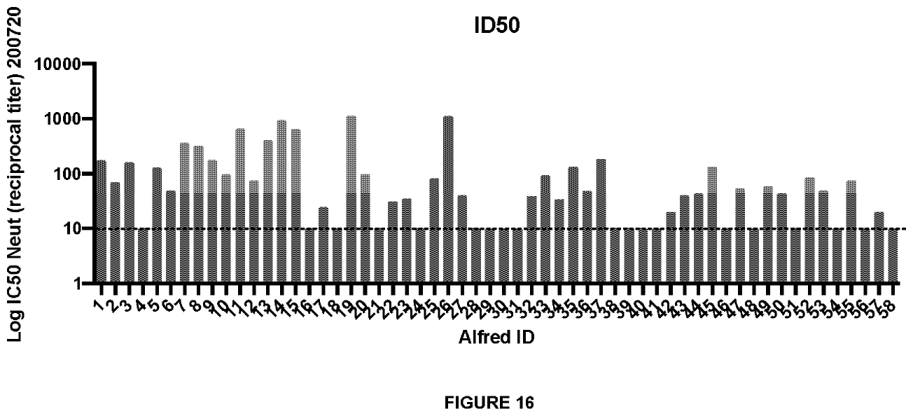

Jackson et al. NEJM (published 14 Jul. 2020 DOI: 10.1056/NEJMoa2022483) describe a pseudotyped lentivirus reporter single-round-of-infection neutralization assay (PsVNA) from the NIH Vaccine Research Centre which was shown to report as well as live wild-type SARS-COV-2 plaque-reduction neutralization testing (PRNT) assay. For example, the ability of lentiviral particles expressing Spike to infect cells expressing high levels of the ACE2 receptor (egg, 293-ACE2, huh7 cells) is measured in the presence or absence of sera and single cycle infection is measured via a luciferase reporter. Results are presented as % entry versus reciprocal dilution of plasma/serum as shown in FIG. 15 and FIG. 16.

Neutralizing assays for non-viral infections and for toxins or venoms are known in the art. For example, neutralizing antibodies against Clostridium difficile toxins is described in Xie et al. Clin. Vaccine Immunology 20(4):517-25, 2013.

In one embodiment, subject samples that test positive for a particular level, such as a high level of pathogen specific dIgA may be further tested by neutralizing assay. Tests may be performed on the same of different samples. However, the methods of the present invention provide a strong indication that pathogen specific dIgA is strongly neutralizing and therefore additional neutralizing tests are optional.

### Convalescent Blood Products

As used herein “convalescent” refers to a person who is recovering after an illness and does not pose a transmission risk. A person skilled in the art will appreciate that in the context of the present application that a convalescent person is recovering from an illness that affects the mucosa of the subject and/or causes a mucosal immune response in a subject or from exposure to a toxin.

Also that when selecting convalescent subjects as blood donors for convalescent plasma therapy or for other elevated dIgA blood products, early convalescence is required because levels of dIgA drop off steeply after about 2-3 months from infection. Ideally potential donors are screened and then, if the subject displays a medium or high level of antigen specific dIgA, such as spike or RBD specific dIgA then that subject is prioritised for apheresis.

Ideally, the subject would present as negative for a diagnostic test for the presence of the pathogen. In the case of a virus, the convalescent person would test negative via e.g. a RNA or DNA PCR or other nucleic acid based test for the virus or antigen test for the virus or infectious virus test for the virus. In an embodiment, when the illness is caused by SARS-CoV-2 the convalescent person would ideally be negative for SARS-COV-2 when assessed via a pathogen nucleic acid based diagnostic test or antigen diagnostic test or infectious virus diagnostic test. Blood products can be treated to remove infectious pathogens for example by filtration.

In light of the present findings that dIgA levels rise early in infection and fall rapidly at about 2-3 months from infection, the ability to identify and take dIgA containing plasma from convalescent subjects quickly is clearly imperative. The present application facilitates this process.

Plasma or a blood fraction from a convalescent person with dIgA levels, or dIgA immunoglobulins purified therefrom, can be used to treat or prevent the same or similar illness in a non-convalescent person (e.g. bodily fluids from a SARS-COV-2/COVID19 convalescent person can be used to treat or prevent SARS-COV-2/COVID19 in a SARS-CoV-2/COVID19 non-convalescent person). This is referred to as “convalescent therapy”. In an embodiment, the blood or plasma will comprise a plurality of antibodies comprising one or more pathogen specific dIgA antibodies, including one or more dIgA neutralizing antibodies to a specific mucosal pathogen or toxin. In an embodiment, the bodily fluid is blood or a fraction thereof. In an embodiment, the blood fraction is plasma. In an embodiment, the blood fragment is serum. In an embodiment, the blood fraction is a purified immunoglobulin (e.g. purified dIgA) that may be administered as a pharmaceutical composition

In an embodiment, the convalescent therapy is convalescent plasma therapy. In an embodiment, the convalescent therapy is convalescent serum therapy.

In an embodiment, as determined herein circulating fluid from subjects recovering from a mucosal viral infection may comprise highly neutralising dIgA antibodies and therefore convalescent therapy derived from such subjects comprises neutralizing dIgA antibodies. In an embodiment, the convalescent therapy comprises administration of plasma comprising a high level of neutralizing dIgA antibodies. In one embodiment, a high level is determined through comparison with other convalescent patients having had the same pathogen or strain/variant to determine a reference high level. In another embodiment, the level of Spike specific vs nucleocapsid specific dIgA is determined and a reference threshold determined therefrom. In an embodiment, the neutralizing antibody is an antibody specific to a mucosal pathogen or toxin, or is an antigen binding derivative thereof.

In an embodiment, the convalescent therapy is formulated as a pharmaceutical composition.

Convalescent therapy can be used to prevent illness in a subject that is a member of a high risk population e.g. an emergency worker or subject with an underlying condition that may make them particularly susceptible to severe symptoms associated with the disease (e.g. subjects with high risk of subsequent deterioration, such as, those aged above 70 or dependence on oxygen with a baseline oxygen saturation of less than 94%).

Convalescent therapy can be used to treat illness in a subject e.g. by reducing one or more of: the severity of one or more symptoms in a subject; the duration of one or more symptoms in a subject; the severity or duration of the disease course in a subject; the risk of requiring ventilator assistance; the time of hospitalisation; the time spent in an intensive care unit; the incidence of organ failure and mortality.

In an aspect, the present invention provides a method for identifying convalescent human subjects with high levels of pathogen-specific neutralizing antibody for prioritisation as plasma donors for convalescent plasma therapy, said method comprising measuring the level of pathogen specific immunoglobulin or pathogen specific dIgA in the plasma.

In an aspect, the present invention provides a method for identifying convalescent human subjects with high levels of toxin neutralizing antibody for prioritisation as plasma donors for convalescent plasma therapy, said method comprising measuring the level of toxin specific immunoglobulin or toxin specific dIgA in the plasma.

In an aspect, the present invention provides a method for identifying convalescent human subjects with high levels of venom neutralizing antibody for prioritisation as plasma donors for convalescent plasma therapy, said method comprising measuring the level of venom specific immunoglobulin or venom specific dIgA in the plasma.

In an aspect, the present invention provides a method for identifying in a non-human animal, levels of venom neutralizing antibody, said method comprising measuring the level of venom specific immunoglobulin or venom specific dIgA in a blood sample therefrom. Animals producing high levels of dIgA can be selected. dIgA can be purified from selected animals using a dIgA specific binding reagent as described herein, such as CSC, taking advantage of the cross-species binding affinity of pIgR as described in WO 2014/071456 (incorporated in full by reference herein)

In an embodiment, the bodily fluid or fraction thereof is collected from subjects identified using the methods as described herein weekly, every week and a half, every 2 weeks or every three weeks. In an embodiment the bodily fluid or fraction thereof is collected from subjects identified using the methods as described herein for about a month, or for about two months, or for about three months.

In an aspect, the present invention provides a method of producing a pharmaceutical composition comprising an antibody specific to a mucosal pathogen or toxin or an antigen binding derivative thereof, wherein at least 80% of the antibody or derivative is dIgA isotype (comprises one or more dIgA constant regions) comprising:
i) selecting one or more convalescent human subjects with high levels of the pathogen-specific or toxin-specific neutralizing dIgA antibody using the method as described herein; and
ii) obtaining a bodily fluid from one or more human subjects with high levels of the pathogen-specific or toxin-specific neutralizing dIgA antibody.

A person skilled in the art will appreciate that the bodily fluid for the convalescent therapy can be collected by an apheresis procedure. In an embodiment, the bodily fluid is collected from a single donor. In an embodiment, the bodily fluid is collected from one or more donors.

The bodily fluid may undergo one or more processing steps to separate a specific fraction of the bodily fluid for use as a convalescent therapy e.g. plasma or serum.

This may include centrifugation to collect the plasma and/or serum.

In an embodiment, an exogenous anti-coagulation and/or preservative is added to the bodily fluid or fraction thereof.

In an embodiment, the bodily fluid or fraction thereof is freeze dried, lyophilized, concentrated, affinity purified and/or separated.

In an embodiment, the bodily fluid or fraction thereof is concentrated to increase the neutralizing dIgA antibody concentration. Suitable methods employ affinity purification using a dIgA binding agent as described herein.

The bodily fluid or fraction thereof may be tested to confirm the subject is convalescent (i.e. to confirm the absence of a specific pathogen or toxin). This may include for example a PCR test for a pathogen DNA or RNA, an antibody assay, a toxin assay, a viral plaque forming assay. In an embodiment, the subject is pre-screened before collection of the body fluid or fraction thereof to confirm the subject is in a convalescent state and does not pose an infection risk.

In an embodiment, the subject is pre-screened before collection of the bodily fluid or the bodily fluid or fraction thereof is tested from the presence of one or more other pathogens or disease states that could cause illness in a recipient. For example, the bodily fluid or fraction thereof may be tested for one or more of: Human Immunodeficiency Virus (HIV) 1, HIV2, Hepatitis B virus, Hepatitis C virus, blood cancer (leukemia, lymphoma, Hodgkin's disease), Syphilis, Human T-cell lymphotropic virus (HTLV), Cytomegalovirus, Malaria, red blood cell antibodies and anti-HLA antibodies.

In an embodiment, the bodily fluid or fraction thereof is tested for ABO and RhD blood groups.

In an embodiment, the bodily fluid or fraction thereof is formulated as a pharmaceutical composition.

In an embodiment, the pharmaceutical composition comprises a high level of neutralizing antibody. In an embodiment, the neutralizing antibody is an antibody specific to a mucosal pathogen or toxin, or an antigen binding derivative thereof. In an embodiment, the neutralizing antibody is dIgA.

In an embodiment, neutralizing antibody titre of the pharmaceutical composition as described herein is confirmed. In an embodiment, the dIgA neutralizing antibody titre of the pharmaceutical composition as described herein is confirmed.

In one embodiment, the composition has a high level of RBD specific dIgA as determined by the present methods.

In an embodiment, the pharmaceutical composition has a neutralizing antibody titre ID50 of about 200 to about 1000. In an embodiment, the pharmaceutical composition has a neutralizing antibody titre ID50 of about 300 to about 900. In an embodiment, the pharmaceutical composition has a neutralizing antibody titre ID50 of about 300 to about 800. In an embodiment, the pharmaceutical composition has a neutralizing antibody titre ID50 of about 300 to about 700. In an embodiment, the pharmaceutical composition has a neutralizing antibody titre ID50 of about 400 to about 500. In an embodiment, the pharmaceutical composition has a neutralizing antibody titre ID50 of at least about 200. In an embodiment, the pharmaceutical composition has a neutralizing antibody titre ID50 of at least about 300. In an embodiment, the pharmaceutical composition has a neutralizing antibody titre ID50 of at least about 400. In an embodiment, the pharmaceutical composition has a neutralizing antibody titre ID50 of at least about 500.

In an embodiment, the pharmaceutical composition has a dIgA neutralizing antibody titre ID50 of about 200 to about 1000. In an embodiment, the pharmaceutical composition has a dIgA neutralizing antibody titre ID50 of about 300 to about 900. In an embodiment, the pharmaceutical composition has a dIgA neutralizing antibody titre ID50 of about 300 to about 800. In an embodiment, the pharmaceutical composition has a dIgA neutralizing antibody titre ID50 of about 300 to about 700. In an embodiment, the pharmaceutical composition has a dIgA neutralizing antibody titre ID50 of about 400 to about 500. In an embodiment, the pharmaceutical composition has a dIgA neutralizing antibody titre ID50 of at least about 200. In an embodiment, the pharmaceutical composition has a dIgA neutralizing antibody titre ID50 of at least about 300. In an embodiment, the pharmaceutical composition has a dIgA neutralizing antibody titre ID50 of at least about 400. In an embodiment, the pharmaceutical composition has a dIgA neutralizing antibody titre ID50 of at least about 500.

In an embodiment, the pharmaceutical composition comprises about 40 to 400 mg/ml dIgA or more than 100 units of neutralizing dIgA antibody per volume or mass.

In an embodiment, the convalescent therapy and or pharmaceutical composition is administered in combination with one or more other treatment regimens. Administration may be concurrent and or sequential in either order. In an embodiment, the convalescent therapy and or pharmaceutical composition is administered in combination with an antiviral and/or steroid. In an embodiment, the antiviral is selected from an antiviral agent described in Gordon et al., 2020. In an embodiment plasma is taken from the subject within one month of infection or first symptoms.

In an embodiment, the convalescent therapy or pharmaceutical formulation is active for at least at least two weeks, or at least 1 month, or at least 2 months or at least 3 months after administration.

### Toxins

In one embodiment, the pathogen is a toxin. A person skilled in the art will appreciate that the toxin as described herein can be any toxin that elicits the production of neutralizing antibodies in a host. In an embodiment, the toxin is from a biological source. In an embodiment, the toxin is an endotoxin, exotoxin, enterotoxin, mycotoxin, phycotoxin, phytotoxin or a zootoxin. In an embodiment, the toxin is a venom. In an embodiment, the toxin is a venom from a land or a marine organism. In an embodiment, the organism is a snake. In an embodiment, the organism is a spider. In an embodiment the organism is a scorpion.

In an embodiment, the toxin is a protein. In an embodiment, the toxin is a lipopolysaccharide. In an embodiment, the toxin is produced by a bacteria, fungi, algae, plant or animal. In an embodiment, the toxin is produced by a bacteria. In an embodiment, the toxin is produced by a gram positive bacteria. In an embodiment, the toxin is produced by a gram positive bacteria.

In an embodiment, the toxin is from Clostridium. Clostridium perfingens. Bacillus anthracis. Francisella tularensis. Yersinia pestis. Escherichia coli, and Staphylococcus aureus.

In an embodiment, the toxin is selected from diphtheria toxin, Shiga toxin, pertussis toxin, anthrax toxin (lethal factor (LF) and edema factor (EF)), ricin toxin, arbin toxin, T-2 toxin, Staphylococcal enterotoxin B, Staphylococcal enterotoxin F, anthrax toxin, tetanus tocin, saxitoxin, Streptolysis S, taipoxin, tetrodotoxin, viscumin, volkensin, and botulinum toxin.

In an embodiment, the botulinin toxin is one or more of botulinin toxin A, botulinin toxin B, botulinin toxin C1, botulinin toxin C2, botulinin toxin D, botulinin toxin E, and botulinin toxin F.

Although heterologous antivenoms are the main effective treatment for snakebite envenomings, they can cause unwanted side effects such as anaphylactic reactions. Antivenoms are composed of total immunoglobulins (mainly IgG and IgM) or antigen-binding fragments (F(ab′)₂s or Fabs) raised against whole venom(s) via immunization of a host animal. In accordance with the present invention it is proposed in one embodiment to purify dIgA that is venom or antigen/peptide specific from immunised animals to provide antivenom compositions suitable for administration as therapy. In another embodiment, recombinant dIgA forms of antibodies or their derivatives may be engineered and expressed in suitable cells. Here the advantage is physical removal of the toxin via excretion bound to dIgA. In particular, the antibody does not need to completely neutralise the activity of the toxin as it may be excreted in a still active form. For some toxins like botulinum toxins, they have such a long half-life that it is better to remove than to neutralise. Biological samples may be tested using any of the herein disclosed and claimed assays, kits and methods to measure the level or determine the presence of dIgA in the biological samples. Such assays are useful inter alia for screening vaccines for their ability to engender a mucosal immune response as described herein.

Making and Using dIgA Antibodies

In one embodiment, the presence or amount of dIgA is determined by testing for one or more characteristics of dIgA or derivative, such as molecular mass, sequence, MALTI-TOF-MS, isoelectric point, immunoglobulin structure or function, nucleic acid sequence/RNA levels, amino acid sequence, glycosylation, antigen specificity, antibody or ligand specificity.

In one embodiment, nucleic acid molecules, expression vectors, plasmids and heterologous nucleic acids may be prepared by using recombinant DNA technology methods. The availability of nucleotide sequence information enables preparation of isolated nucleic acid molecules as proposed herein by a variety of means. For example, nucleic acid sequences encoding a therapeutic protein can be made using various standard cloning, recombinant DNA technology, via cell expression or in vitro translation and chemical or biological synthesis/interaction techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library. Nucleic acids may be maintained as DNA in any convenient cloning vector. For example, clones can be maintained in a plasmid cloning/expression vector, such as pBluescript (Stratagene, La Jolla, Calif.), which is propagated in a suitable E. coli host cell. Alternatively, nucleic acids may be maintained in vector suitable for expression in mammalian cells. The structure function relationship in IgA is gradually being established (see IgA: Structure, function, Developability Sousa-Pereira et al Antibodies (Basel) 8(4):57, 2019.

A process for preparing a physiological composition enriched for dIgA (multimeric IgA) from blood or an antibody comprising part thereof obtained from a person recently infected with a pathogen, the method comprising affinity purifying dIgA using pIgR that binds to dIgA and substantially fails to bind to IgM, and eluting dIgA to form a pharmaceutically acceptable or physiological composition.

The process wherein the antibody comprising part is serum or plasma.

In one embodiment, the present specification provides a physiological composition obtained or obtainable by the process.

A physiological composition wherein at least 10% to 90% including any figure in between of antigen or pathogen specific immunoglobulin is dimeric or multimeric IgA.

A method of producing a pharmaceutical composition comprising an antibody or a plurality of antibodies specific to a mucosal pathogen or toxin or an antigen binding derivative thereof, wherein the antibody or derivative is dIgA isotype (comprises dIgA constant regions) comprising:
i) selecting convalescent human subjects with high levels of the pathogen-specific neutralizing antibody using the methods as described herein detecting dIgA and
ii) obtaining plasma from the human subjects with high levels of the pathogen-specific neutralizing/dIgA antibody.

A “conservative amino acid substitution” is one in which the naturally or non-naturally occurring amino acid residue is replaced with a naturally or non-naturally occurring amino acid residue having a similar side chain. Families of amino acid residues having similar side chains have been defined in the art. These families include amino acids with basic side chains (e.g., Lys, Arg, His), acidic side chains (e.g., Asp, Glu), uncharged polar side chains (e.g., Gly, Asn, Gln, Ser, Thr, Tyr, Cys), nonpolar side chains (e.g., Ala, Val, Leu, Ile, Pro, Phe, Met, Trp), beta-branched side chains (e.g., Thr, Val, Ile) and aromatic side chains (e.g., Phe, Trp, His). Thus, a predicted nonessential amino acid is replaced with another amino acid residue from the same side chain family. Other examples of acceptable substitutions are substitutions based on isosteric considerations (e.g. norleucine for methionine) or other properties (e.g. 2-thienylalanine for phenylalanine). This term is known in the art and well recognised. The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base {e.g., A, T, C, G, U) or the identical amino acid residue (e.g., Ala, Pro, Ser, Thr, Gly, Val, Leu, lie, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, California, USA) using standard defaults as used in the reference manual accompanying the software. Amino acid sequence identity may also be determined using the EMBOSS Pairwise Alignment Algorithms tool available from The European Bioinformatics Institute (EMBL-EBI), which is part of the European Molecular Biology Laboratory. This tool is accessible at the website located at www.ebi.ac.uk/Tools/emboss/align/. This tool utilizes the Needleman-Wunsch global alignment algorithm (Needleman and Wunsch, 1970). Default settings are utilized which include Gap Open: 10.0 and Gap Extend 0.5. The default matrix “Blosum62” is utilized for amino acid sequences and the default matrix.

The term sequence “similarity” refer to the percentage number of amino acids that are identical or constitute conservative amino acid substitutions as defined in Table 2 below. Similarity may be determined using sequence comparison programs such as GAP (Deveraux et al, 1984 Nucleic Acids Research 12: 387-395). In this way, sequences of a similar or substantially different length to those cited herein might be compared by insertion of gaps into the alignment, such gaps being determined, for example, by the comparison algorithm used by GAP. Derived from includes directly derived from and indirectly derived from. For example dIgA from plasma cells may be cloned and subsequently administered.

### Pharmacological Compositions

In accordance with these embodiments, the antibody composition is generally administered for a time and under conditions sufficient to effect a functional mucosal immune response.

The compositions of the present invention may be administered as a single dose or application. Alternatively, the compositions may involve repeat doses or applications, for example the compositions may be administered 2, 3, 4, 5, 6, 7, 8, 9, 10 or more times.

A “pharmaceutically acceptable carrier and/or a diluent” is a pharmaceutical vehicle comprised of a material that is not otherwise undesirable i.e., it is unlikely to cause a substantial adverse reaction by itself or with the active composition. Carriers may include all solvents, dispersion media, coatings, antibacterial and antifungal agents, agents for adjusting tonicity, increasing or decreasing absorption or clearance rates, buffers for maintaining pH, chelating agents, membrane or barrier crossing agents. A pharmaceutically acceptable salt is a salt that is not otherwise undesirable. The agent or composition comprising the agent may be administered in the form of pharmaceutically acceptable non-toxic salts, such as acid addition salts or metal complexes.

For oral administration, the compositions can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. Tablets may contain a binder such as tragacanth, corn starch or gelatin; a disintegrating agent, such as alginic acid; and a lubricant, such as magnesium stearate. If desired, tablets may be sugar-coated or enteric-coated by standard techniques. The active composition can be encapsulated to make it stable to passage through the gastrointestinal tract. See for example, International Patent Publication No. WO 96/11698.

For parenteral administration, the composition may be dissolved in a carrier and administered as a solution or a suspension. For transmucosal or transdermal (including patch) delivery, appropriate penetrants known in the art are used for delivering the composition. For inhalation, delivery uses any convenient system such as dry powder aerosol, liquid delivery systems, air jet nebulizers, propellant systems. For example, the formulation can be administered in the form of an aerosol or mist. The compositions may also be delivered in a sustained delivery or sustained release format. For example, biodegradable microspheres or capsules or other polymer configurations capable of sustained delivery can be included in the formulation. Formulations can be modified to alter pharmacokinetics and biodistribution. For a general discussion of pharmacokinetics, see, e.g., Remington's Pharmaceutical Sciences, 1990 (supra). In some embodiments the formulations may be incorporated in lipid monolayers or bilayers such as liposomes or micelles. The actual amount of active agent administered and the rate and time-course of administration will depend on the nature and severity of the disease. Prescription of treatment, e.g. decisions on dosage, timing, etc. is within the responsibility of general practitioners or specialists and typically takes into account the condition of the individual patient, the site of delivery, the method of administration and other factors known to practitioners. Examples of techniques and protocols can be found in Remington's Pharmaceutical Sciences, 1990 (supra). Prime-boost immunization strategies as disclosed in the art are contemplated. See for example International Publication No. WO/2003/047617. Thus, compositions may be in the form inter alia of plasma, blood products, serum, purified antigen specific dIgA, recombinant antigen specific dIgA, recombinant antigen specific SIgA.

In another embodiment, the present application provides a method of treating or preventing an infection in a subject, comprising administering to the subject an effective amount of a pathogen or pathogenic antigen specific dIgA antibody to down regulate the level or activity of a pathogen or pathogenic antigen bound by the dIgA at the mucosa. In some embodiments, the subject has a severe infection with a pathogen or is suspected of being at risk from a pathogen. In some embodiments, a combination of antibodies with activity are administered. In some embodiments, the antibodies are neutralizing antibodies.

As used herein, an “effective amount” is an amount of a blood product, agent or composition that alleviates, totally or partially, the pathophysiological effects of infection or other pathological indication of the invention. Unless otherwise indicated, the agent or composition is administered at a concentration that is a therapeutically effective amount. A therapeutically effective amount can also be an amount that is given prophylactically thereby inhibiting any pathophysiological effects of infection, or other pathological indication of the invention. A therapeutically effective amount may depend upon, for example, subject size, gender, magnitude of the associated disease, condition, or injury, and genetic or non-genetic factors associated with individual pharmacokinetic or pharmacodynamic properties of the administered agent or composition. For a given subject in need thereof a therapeutically effective amount can be determined by the presiding medical practitioner.

As used herein, “treat” and all its forms and tenses (including, for example, treat, treating, treated, and treatment) refer to both therapeutic treatment and prophylactic or preventative treatment. A subject in need of treatment includes those already with an infection by the pathogenic organism who are a risk to other individuals, or those at risk from infection and desirous of inhibiting, reducing, preventing etc the infection, disease or transmission of the disease, infection. Reference to “alleviating,” “inhibiting,” “reducing,” or “prevention,” or any variation of these terms, includes any measurable decrease or complete inhibition to achieve a desired result. Conversely, reference may be made to decreasing mortality or morbidity caused by an epidemic or pandemic pathogen. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of disease activity, severity of disease, time to recovery, or transmission compared to normal/no treatment. Alternatively, treatment provides in the recipient an increase in the level of antigen specific dIgA or plasma cells producing same capable of facilitating an effective mucosal immune response against the pathogen. In one embodiment, treatment provides in the recipient an increase in the level of circulatory antigen specific dIgA or plasma cells producing same capable of facilitating an effective mucosal immune response against the pathogen.

The decision to use antivenom is based on the patient's history, examination and pathologic findings, and the type of antivenom used will depend on geographic, clinical, pathologic factors and the exigencies of the situation.

### FURTHER DESCRIPTIONS OF EMBODIMENTS

Respiratory infections, including SARS coronaviruses, trigger secretion of IgA, the most abundant antibody isotype produced in humans, and a major component of the mucosal immune response. IgA exists as two different isoforms, IgA1 and IgA2, with IgA2 having at least two different allotypes in the human population. In plasma, around 90% of IgA is monomeric and primarily of the IgA1 isotype, while around 10% is dimeric (dIgA), comprising two IgA monomers connected via the J chain. Although dIgA is a relatively minor component of plasma, it is the direct precursor of secretory IgA (SIgA) which is exported in large amounts on mucosal surfaces. Transport of dIgA across mucosal surfaces is performed by binding to the polymeric Ig receptor (pIgR) on the basolateral surface of epithelial cells, where it is transcytosed and the pIgR is then cleaved at the apical surface to release SIgA, representing dIgA covalently bound to secretory component (SC), the extracellular domain of pIgR. dIgA in plasma and SIgA at mucosal surfaces can exert multiple functions to protect against pathogens or toxins. These include direct neutralization that blocks pathogen entry or toxin activity, or binding and export of pathogen or toxin, or binding to effector cells to activate cellular processes to enhance pathogen or toxin clearance.

The ability to effectively detect early infections or seroconversion is critical to their control as shown recently with SARS-COV-2. During the SARS COV-2 pandemic it has been determined that RT-PCR viral RNA detection is most sensitive during the acute phase of viral infection and replication but wanes rapidly due to falling amounts of detectable viral RNA. Antigen detection of SARS-COV-2 is considered to be about 80% as sensitive as RT-PCT over the same time scale. Detection of viral nucleocapsid antigen follows similar kinetics to RT-PCR but with reduced sensitivity. Antigen specific IgG and IgM antibody testing is considered to be unreliable for discriminating recent infections (e.g. less than 3 months) from more distant infections (e.g. more than 3 months). IgM levels are very variable and IgG levels persist for longer periods generally precluding their use in directly determining a recent infection. Levels of IgM have not been found to be reliable or sensitive markers of recent infection. In accordance with the present application, dIgA detection of recent infection fills the current gap in methods for population or contact screening and is particularly useful after or during the tail of the acute phase of infection when viral RNA is not readily detectable by standard RT-PCR assay and viral antigen is not detectable by standard ELISA or other assays, leading to false negative screening results, yet before IgG is reliably detectable.

For example, longitudinal population based studies described herein show approximately half of PCR positive symptomatic subjects were positive for dIgA within 0-5 days of symptom onset, while none were IgG or IgM positive using commercially available tests. Sensitivity for dIgA increased to 65% by days 6 to 10 and was 100% sensitive on days 11 to 15 post symptom onset (Table 3). Further, dIgA levels typically remain high for up to 2 to 3 months after virus levels drop before dIgA rapidly disappears from the circulation, providing further information concerning the recency of infection.

Accordingly, as described and enabled herein antigen specific dIgA screening provides advantages over other antibody isotypes for detecting recent infections.

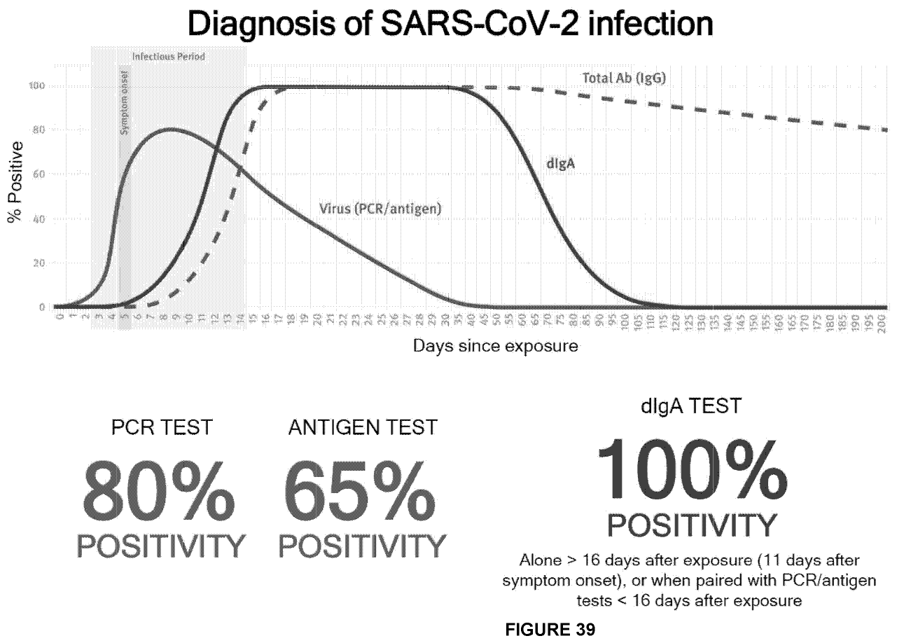

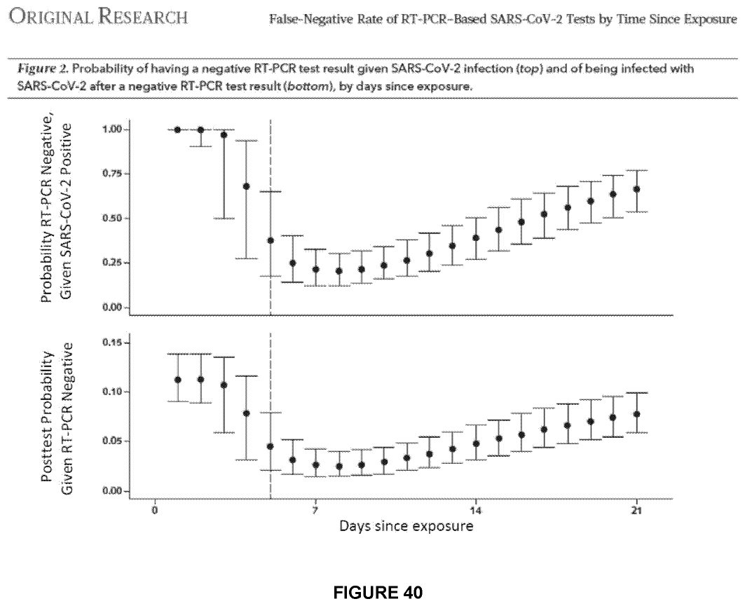

Others have shown the increasing probability of false negatives by RT-PCR or antigen testing as days from symptom onset (taken with COVID-19 to be 5 days from exposure) increase (See FIG. 40 Kucirka et al. Ann Intern Med. doi:10.7326/M20-1495). As described herein, and illustrated in FIG. 39, dIgA screening delivers an ability to accurately detect a recent infection at a time post infection when molecular or antigen testing is showing diminished accuracy. As noted, although IgG levels only lag behind dIgA by a few days, its long half life precludes detection of recent infection, unlike the case for dIgA which displays a rapid decline after virus levels drop, between 40 and 100 days post exposure for SARS-COV-2. In addition, the presence of IgG in vaccinated individuals would preclude its use for detecting “breakthrough” infections that may occur with waning levels of immunity and/or exposure to viral variants. This Figure also illustrates the complementary times scales for efficient infection detection and the advantage of combining dIgA testing with molecular or antigen tests (PCR/antigen) to detect more recent infections than either test alone.

Accordingly, as described and enabled herein antigen specific dIgA screening provides advantages over other antibody isotypes for detecting recent infections in the post-acute phase of viral replication for a reduced risk for failing to detect and infected.

Accordingly, the present application provides an in vitro assay or kit for detecting or excluding the presence of a complex comprising (i) dIgA from within a subject's blood sample (e.g, whole blood, plasma, serum), and (ii) a pre-prepared infectious agent or toxin or an antigen of an infectious agent or toxin, and/or (iii) a SC (secretory component)-based protein that specifically forms a complex with dIgA and not with IgA or IgM or IgG from within the blood sample.

In another aspect, the present application provides an in vitro assay or kit for detecting or excluding the presence of a complex comprising (i) dIgA from within a subject's blood sample (e.g, whole blood, plasma, serum), and (ii) a pre-prepared respiratory virus or an antigen of a respiratory virus, and/or (iii) a SC (secretory component)-based protein that specifically forms a complex with dIgA and not with IgA or IgM or IgG from within the blood sample.

Accordingly, the present application provides an in vitro assay or kit for detecting or excluding the presence of a complex comprising (i) dIgA from within a subject's blood sample (e.g, whole blood, plasma, serum), and (ii) a pre-prepared coronavirus or an antigen of a coronavirus, and/or (iii) a SC (secretory component)-based protein that specifically forms a complex with dIgA and not with IgA or IgM or IgG from within the blood sample.

In one embodiment, the secretory component based protein is chimeric secretory component (CSC) as described herein.

In one embodiment, the antigen is a vaccine antigen.

In one embodiment, the antigen is not a vaccine antigen. Specifically, in one embodiment, testing undertaken in vaccinated subjects may use a different antigen to that contained within the vaccine.

In one non-limiting embodiment, the coronavirus antigen is a Spike protein antigen.

In one embodiment, the assay or kit is highly sensitive for antigen specific dIgA within 2-3 or 0-4 weeks of symptom onset or exposure.

In one embodiment, the assay or kit is 95% to 100% sensitive by days 11 to 15 after symptom onset (16 to 20 days after exposure).

In one embodiment, the assay or kit is moderately to above-moderately sensitive by 1 and 2 weeks post symptom onset.

In one embodiment, the assay or kit is approximately 65% sensitive by days 6 to 10 after symptom onset (11 to 15 days after exposure).

In one embodiment, the assay or kit is approximately 49% sensitive (low-moderate sensitivity) within one week of symptom onset (5 to 10 days after exposure).

In one embodiment, the assay or kit is 49% sensitive by 0 to 5 after symptom onset (by 5 to 10 days after exposure). In contrast IgM and IgG antibody tests were negative during this period.

As shown herein dIgA levels are detected up to about 100 days post symptom onset (symptom onset, if it occurs, is about 5 days after exposure).

As shown in Table 3, when different antibody classes are compared peak dIgA levels were reached and maintained earlier after symptom onset than either IgM or IgG.

While the precise timing described herein and the utility of CSC-dIgA testing have been established by longitudinal studies during the SARS-COV-2 pandemic, the features of early dIgA responsiveness, peak levels extending from the post-acute phase of virus replication and the predictable tailing off of circulating dIgA are considered to provide new and valuable tools for serological assessments. Even within coronavirus, variants, such as Delta variants, will alter the period wherein dIgA is observed, as with different variants subjects become get sick at different rates and have symptoms for different periods. Individuals who vary from the norm do not invalidate the model.

In one embodiment, the assay or kit providing CSC-dIgA complex detection is highly sensitive from week 1 or 2 post CoV symptom onset and the presence of the complex remains a highly specific sensitive marker for recent infection through to the drop in dIgA levels at about two to three months from exposure.

In one embodiment, the assay or kit providing CSC-antigen specific dIgA complex detection is highly sensitive from week 1 or 2 post respiratory virus symptom onset and the presence of the complex remains a highly specific sensitive marker for recent infection through to the drop in dIgA levels at about two to three months from exposure or the drop in viral levels.

In one embodiment, the symptoms are symptoms of a respiratory virus and exposure is to a respiratory virus.

In one embodiment, the symptoms are symptoms of a coronavirus and exposure is to a coronavirus.

In one embodiment, the assay or kit is at least 95% to 100% sensitive, at least 96%, at least 97%, least 98% or at least 99% sensitive or 100% sensitive for subjects in the post-acute phase of a recent respiratory viral infection. dIgA screening can sensitively identify antigen specific dIgA in people for two to three months after symptom onset. DIgA levels decline quite rapidly which makes it, in part, a good marker for a recent infection i.e., within about 100 days.

In one embodiment, the post-acute infection phase begins when molecular (eg, RNA) assays and antigen based assays for viral detection decline in sensitivity as viral levels decline in sampled material. The post-acute infection phase may be determined for each infection agent and may commence from day 1 to day 100 post symptom onset or from detection of an infectious agent. Further, in one embodiment, the post-acute phase of a recent infection commences before IgG is reliably detectable or well before IgG testing approaches the same level of sensitivity as dIgA, typically at a later stage after infection than dIgA. As IgG levels persist for a longer time period than dIgA, IgG does not display the temporal sensitivity required for use as a marker for recent infection with a respiratory virus.

Complex detection may be mediated by a large and diverse range of methods as described herein or as known in the art of biological assays and biological kits/devices, lateral flow immunoassays etc and may involve visual or non-visual detection, signal augmentation, and an instrument reader.

Reference to a blood sample means a whole blood sample or plasma, serum or other blood derivatives comprising circulating antibodies or antigens. Reference to “a” blood sample includes more than one blood sample unless explicitly stated otherwise. DIgA is also detected in saliva.

In one embodiment, the present assays for population based management are for use in detecting the earliest seroconversion or the earliest reliable detection of a recent infection in subjects who have symptoms of a contagious respiratory infection or those who have neither symptoms or detectable viral levels.

In one embodiment, the present assays for population based management are for use in detecting the earliest seroconversion or the earliest reliable detection of infection in subjects who have no overt symptoms or signs of a contagious respiratory infection.

SC-dIgA-antigen complex-based screening of blood is proposed for a respiratory viral infection in a population to detect recent active infections. In one particular embodiment, screening permits detection during a post-acute infection phase when molecular (RT-PCR) or antigen testing displays false negatives due to falling virus numbers in respiratory mucosa (e.g., swab or plasma).

An in vitro method for screening blood samples and detecting recent active viral respiratory infection at a time post-acute infection when molecular tests (RT-PCR and antigen) and non-dIgA antibody tests display limited sensitivity and specificity for recent infection.

Complex detection may be mediated by a range of methods including those described herein or known in the art of biological assays and may involve visual or machine detection, signal augmentation, and an instrument reader.

In one embodiment, the assay or kit comprises a chimeric (e.g., rabbit/human) form of the polymeric Ig receptor, CSC, to bind with high specificity to dimeric IgA, thereby immobilising complexes of antigen-specific dIgA and SARS-COV-2 RBD or other antigen. In one embodiment, the assay or kit is based on the ability of a chimeric (rabbit/human) form of the polymeric Ig receptor, CSC, to bind with high specificity to dimeric IgA, thereby immobilising complexes of antigen-specific dIgA and SARS-COV-2 RBD antigen-colloidal gold.

In one embodiment, the kit or strip or device comprises a means for reducing red blood cell entry into the binding reaction zones of the device.

In one aspect, dIgA complex screening of a sample is combined with testing for the presence in a blood sample of one or more other antibody isotypes, such as, but not limited to IgG, IgA and IgM directed against the respiratory virus. In one embodiment, testing is for dIgA and IgG. In one embodiment, testing is for dIgA and IgA. In one embodiment testing is for dIgA and IgM. Further testing or controls includes testing for total immunoglobulin. The results for further tests may serve as controls and/or provide further information concerning the recency of infection. Testing may be conducted using the same blood sample or separate blood samples. Testing for combined antibodies in kit formats may be conducted using the same or separate test strips/devices/portions. Testing may be qualitative or quantitative or semi-quantitative or semi qualitative.

In another aspect, the results of dIgA complex screening is combined with the results of antigen testing or molecular (nucleic acid based) testing to further improve screening methods.

Diagnostic/serology assay results may be stored on a computer and delivered to individuals or organisations around the world or to specific sites.

In one embodiment molecular testing for an infectious agent is nucleic acid testing by any method known in the art. Once the nucleic acid sequence of a relevant portion of the genome or gene is known primers and probes can be readily developed. SARS-COV-2 molecular testing is typically by PCR of DNA generated by reverse transcription of viral RNA using primers and probes specific for a gene of the virus such as RdRP, E gene, N gene etc. Assays may be quantitative or qualitative or semi quantitiative or semi qualitative. Isothermal nucleic acid amplification tests (e.g., loop-mediated isothermal amplification (LAMP) may also be employed, or CRISPR-based detection methods as recognised in the art. PCR based molecular testing may be conducted in a laboratory or at point of care using laboratory independent devices. Screening methods may include genomic sequencing of all or part of the respiratory virus or other molecular assays to distinguish between strains.

Kits for molecular assays may be similar to laboratory molecular tests but use portable versions of laboratory equipment as known in the art.

Biological samples from molecular testing will depend upon the infections agent or toxin. Respiratory virus material may be detected in nucleic acid material purified from virus in secretions from biological samples for example, sputum, nasal, throat, nasopharangeal or oropharyngeal swabs.

In one embodiment, antigen testing is for any antigen of the infectious agent as described herein and known in the art. In the case of viruses, the antigen may be an RBD antigen. Accordingly, in one embodiment, the antigen may be a Spike antigen. In one embodiment the Spike antigen comprises all or part of the S1 subunit. In one embodiment, the antigen is a non-RBD antigen. In one embodiment, the antigen is a CoV nucleoprotein antigen.

Biological samples for antigen sampling include any sample likely to comprise the antigen of interest. Illustrative methods use swab material such as for molecular testing but may also employ blood/plasma/serum samples or swabs from any tissue or material.

In one embodiment, a blood sample is screened for the presence of antigen, such as a viral protein.

In one embodiment, the blood sample is screened for the presence of nucleoprotein antigen.

In one embodiment, one blood sample (the same blood sample) is used for detecting dIgA by SC-dIgA-antigen complex screening and detecting antigen by screening for an antigen-antigen binding molecule complex.

Combined testing for dIgA antibody and antigen in kit/device formats may be conducted within the same, overlapping or separate test strips/devices/portions/compartments.

In one embodiment, the present assays and kits are used to reduce the risk of symptom onset of a contagious agent, such as a respiratory virus, by directing subjects with recent active infections to self isolate, vaccinate, or medicate, or other behavioural changes, to reduce symptoms or signs thereof.

In one embodiment, the present methods and kits are used to reducing forward transmission of a contagious agent by directing subjects identified as having recent or active infections to self isolate, or vaccinate or medicate, or other temporary or long term behavioural change.

In one embodiment, the subject is a human subject.

In one embodiment, the method further comprises an in vitro assay for the presence of a complex comprising (i) antigen of a contagious respiratory virus from within a blood sample of a subject (e.g, whole blood, plasma, serum), and (ii) a pre-determined binding agent, antibody or antibody derivative that specifically recognises the antigen. Complex detection may be mediated by a range of methods such as those known in the art of biological assays and may involve visual detection, signal augmentation, and an instrument reader. The complex may form at a test line.

Illustrative control binding pairs are anti-chicken IgY-chicken IgY gold conjugate.

In one embodiment, the antigen for antigen detection is nucleoprotein antigen of CoV.

In one embodiment, the pre-prepared antigen used in antibody testing (including dIgA testing) is specific for Spike protein or a Spike antigen as defined herein and the pre-prepared binding agent used in antigen testing is specific for nucleoprotein.

The subject rapid assays are developed to increase the availability of testing in populations and individuals, and to decrease the turn-around time relative to standard laboratory tests, to facilitate and speed up population management during epidemics/pandemics and reducing the spread of the contagious agent. Rapid tests are also developed as described herein to gauge, exclude, determine, or monitor the development of a mucosal immune response or the magnitude of the mucosal immune response in vaccinated subjects.

Assay users are able to record results on a computer/smart device, collate data, and forward or display individual or collated data in any suitable format, including for example a dashboard with heat maps, to illustrate individual results, group results, compare results, disease surveillance and testing trends. Data visualization techniques may be used to assist users to understand the epidemiological status of epidemics and mobilize their response more effectively.

The complex or complexes may be detected qualitatively, semi-qualitatively or quantitatively or semi-quantitatively.

In one embodiment, IgG or IgM or IgG and IgM antibody testing is not proposed for early contact screening and the present application provides an antibody test based on specifically detecting dIgA to antigen of the agent which may be rapid, and is reliable and sensitive and specific for recent post-acute phase infections, useful, for example, in contact tracing. IgG or total Ig may be used as controls. Specifically, IgM is typically not a reliable marker producing heterogeneous results, and IgG is produced or present for too long a period to be useful for most contact tracing objectives. IgM/IgG combined testing displays reduced sensitivity compared to dIgA. IgA is typically produced early but levels persist for too long for IgA testing to be directly useful for early contact screening. Thus, for example, as determined herein the signal to cutoff ratio is higher for dIgA testing as described herein than IgM testing which has a low predictive potential.

Accordingly, in one embodiment, specific dIgA testing using pIgR based reagents such as CSC, that only bind dIgA and not monomeric IgA or IgM is employed as described herein to detect recent including post-acute infection. The reliable trajectory of dIgA upwards as viral levels decline, and then downwards after approximately 100 days places dIgA as the antibody biomarker of choice for detecting recent infections, either by itself or together with respiratory virus testing.

In one embodiment wide-spread dIgA testing is deployed to determine; those who have evidence of recent CoV/respiratory virus infection to track chains of transmission, to identify potential common sources of infection and their onward contacts, and to reduce the number of people who need to isolate in order to reduce forward transmission of the virus. In this regard combined testing of samples for antigen specific dIgA and antigen or antigen specific dIgA and molecular testing is show herein to provide better temporal coverage for recent infections than either test alone.

In another embodiment, as dIgA plays an essential role in mucosal protection against respiratory infections or other contagious agents or toxins, detection of circulating dIgA is used to assess a subject's neutralising mucosal antibody response during the testing period.

In another embodiment, as dIgA plays an essential role in mucosal protection against CoV infections, detection of the level of circulating dIgA is used to quantify a subject's recency of CoV infection (within 100 days) and/or magnitude of the neutralising mucosal antibody response to the pathogen.

In another embodiment, as dIgA plays an essential role in mucosal protection against CoV infections, detection of the level of circulating (including salivary) dIgA is used to quantify the subject's mucosal immune response to vaccination.

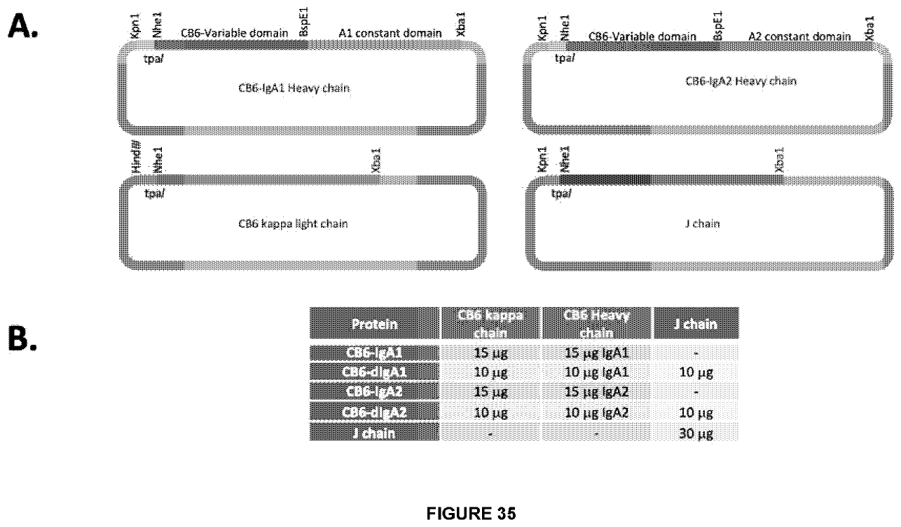

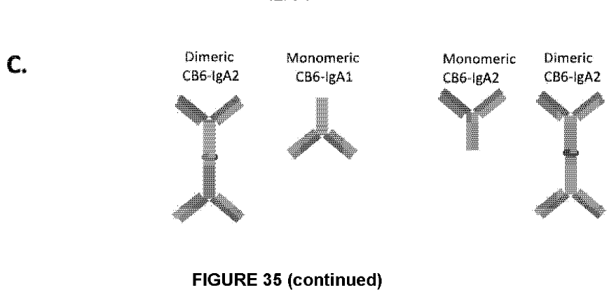

Cells and cell lines Expi293F cells were maintained in suspension culture in Expi293 expression medium (ThermoFisher Scientific) at 37° C. and 8% CO₂. Plasmids encoding proteins of interest were transfected into cells using the ExpiFectamine 293 reagent according to the manufacturer's instructions (ThermoFisher Scientific). In order to biotinylate proteins with a C-terminal Avitag (GLNDIFEAQKIEWHE), Expi293F cells stably transfected with a plasmid directing expression of the BirA biotin ligase (ExpiBirA) were used. For in situ biotinylation, media was supplemented with a final concentration of 50 uM D biotin.
Vectors. The receptor binding domain (RBD) of the SARS COV-2 spike protein (amino acids 332-532) and the chimeric secretory component (CSC) of the polymeric immunoglobulin receptor (650 amino acids protein synthesized by GenScript) were synthesized by ThermoFisher Scientific and subcloned into pcDNA3-based vectors under the control of the CMV promoter. Both proteins encode a C-terminal 6 histidine tag to enable purification by immobilized metal affinity chromatography (IMAC). In addition, the Avitag sequence was appended onto the C-terminus of RBD to enable site-specific biotinylation in ExpiBirA cells.
Protein Expression and purification. After transfection with RBD-encoding plasmid Expi293F or Expi293F BirA cells were incubated at 34° C. whereas Expi293F cells transfected with CSC-encoding plasmid were incubated at 37° C. Four days after transfection tissue culture supernatants were clarified and target proteins were purified by IMAC using Talon metal affinity resin (Clontech Laboratories) following the manufacturer's recommendations. The eluted proteins were subject to gel filtration using a Superdex 200 16/600 column (GE Healthcare) with PBS as the liquid phase. Fractions corresponding to monomeric RBD or dimeric CSC were pooled and concentrated in Amicon Ultra 10 (RBD) or 30 kDa (CSC) devices (Merck) prior to use.
ELISA. Plasma samples were tested in ELISA based assays to determine levels of IgG (EUROIMMUN AG and EDI Inc), and IgM (EDI Inc) according to the manufacturer's instructions. Levels of IgA may be determined using commercial assays, such as EUROIMMUN AG,
Illustrative antibodies and proteins. Recombinant synthetic (GeneArt) chimeric IgA1 and IgA2 heavy chain sequences were constructed by joining the CB6 variable domain (amino acids 1-137) to the CHI domain (amino acids 161-516) of IgA1 (J00220) or the CHI domain (amino acids 161-501) of IgA2 (J00221) via a BspE1 restriction site and cloning into a pcDNA3 vector with an N-terminal TPA leader sequence. For the expression of the J chain the mature protein sequence (DQ884395) amino acids 22-175 was synthesized (GeneArt) and cloned in frame with the tissue plasminogen activator (tpa) leader sequence into pcDNA3 based expression vector (FIG. 35). Recombinant chimeric IgA1 and IgA2 were expressed in 293 Expi cells by co-transfection of the CB6 kappa chain and CB6-IgA1 or CB6-IgA2 heavy chain sequence. Dimeric IgA1 and IgA2 were expressed by co-transfecting CB6 kappa light chain and CB6-IgA1 or CB6-IgA2 heavy chain sequence and pcDNA3-J using equivalent amounts of DNA. After 4 days, supernatant fluid was collected, clarified and either used directly, or purified using 45% ammonium sulfate and size exclusion chromatography on a Superose 6 Increase 10/60 GL column (AKTAPure). Proteins were analyzed on SDS-PAGE and visualized with Coomassie blue. The sequence of the chimeric dIgA-CB6 is set forth in the
SEQ ID NO: 21-24 and represented in FIG. 35.

The construction and expression of recombinant synthetic CSC protein has been previously described by the group. In one embodiment, soluble cSC, 6×Histidine-tagged cSC (cSC-His) and human CD4 cytoplasmic domain (D)-containing cSC (cSC-CD4), human SC (hSC-CD4) and rabbit SC (rSC-CD4) were expressed using modified published methods (Roe M, Norderhaug I N, Brandtzaeg P, Johansen F E. Fine specificity of ligand-binding domain 1 in the polymeric Ig receptor: importance of the CDR2-containing region for IgM interaction. J Immunol. 1999; 162:6046-52). The hSC and rSC sequences were obtained from Genebank NM_002644.3 and X00412.1, respectively. Chimera of rSC-D1/hSC-D2-D5 were generated by splice overlap extension polymerase chain reaction with primers that introduced silent mutations in D1/D2 overlaps, followed by rSC/hSC-D1 exchange using EcoRI and SacI restriction digestion, and cloning in eukaryotic expression vector pCDNA3.1 Zeo (Invitrogen; San Diego, CA). Constructs were confirmed by DNA sequencing. Human embryonic kidney 293T (HEK293T) cells were grown in Dulbecco's Modified Eagle Medium (DMEM)-GlutaMAX, 2.5% foetal calf serum (FCS), 100 U/ml Penicillin and 100 μg/ml Streptomycin (Invitrogen; San Diego, CA). HEK293T cells were transfected with plasmid encoding rSC-D1/hSC-D2-D5 using Lipofectamine 2000 (Invitrogen; San Diego, CA) based on manufacturer's protocol, plus 25 ml DMEM-GlutaMAX+10% FCS+1% Penicillin/Streptomycin. The cSC-containing supernatants were harvested 48-72 h post-transfection and centrifuged to remove cells.

The following reagents were sourced from commercial suppliers: Streptavidin-gold (40 nM) was purchased (Abcam cat #ab186864), Chicken IgY gold conjugate 40 nm (BBI cat #Cont.Chick), SARS-COV-2 RBD (GenScript Cat #Z03483-1), 5D protein stabilizer (Abacus cat #5D82411B). Commercial reagents were used for consistency between comparative assays.

Illustrative Lateral flow assay. The device cassette consists of a plastic housing (Nanjing BioPoint Diagnostics, PR China) with loading wells and a window to read results. Within the cassette is a nitrocellulose membrane strip (Sartorius, Germany), with a conjugate pad containing biotinylated RBD protein complexed with streptavidin-colloidal gold and colloidal gold-conjugated chicken IgY as a procedural control. RBD-biotin (4 ug/mL) was mixed with streptavidin gold conjugate (OD 1.5) for 30 min at room temperature. Chicken IgY gold conjugate (OD 1) and drying buffer (5 mM HEPES, 0.17% PEG, ImM EDTA, 0.5% BSA, 10% trehalose, 0.12% Tween) were added to the mixture and dispensed onto conjugate pads, frozen at −80° C. for 30 min and vacuum dried for 1 h at room temperature. Nitrocellulose membranes (Sartorius, Germany) were striped with proteins in PBS (pH 7.4) to give two test lines, with test line 1 comprising 0.4 μl/cm of CSC (1 mg/ml) to capture dIgA, and test line 2 comprising 0.4 μl/cm of RBD (0.5 mg/ml) to capture total anti-RBD antibody, and a third procedural control line comprising 0.4 μl/cm of rabbit anti-chicken IgY (0.1 mg/ml) and dried at room temperature. Conjugate pads and nitrocellulose membranes were laminated together with absorbent pads at the distal end and cut into 5 mm wide strips before placing into cassettes.

The test specimen (15 μl plasma) is dispensed into the sample well A (plasma port) of the test cassette, rehydrating the gold conjugates, and four drops of running buffer (phosphate buffered saline pH 7.4, 0.5% Tween20, 0.05% Sodium azide) are added to well B (buffer port) of the test cassette, initiating sample flow by capillary action. The test is read after 30 minutes. For whole blood tests, in the absence of fresh patient samples plasma was used that was reconstituted by addition of 15 μl plasma to 15 μl packed red blood cells. The device was modified by addition of a glass fibre soaked in anti-glycophorin A over the sample pad, and both sample and buffer were added to well B (buffer port) while the plasma port (well A) was not used. Whole blood (30 μl) or plasma (15 μl) was added to the buffer port with the addition of four drops of running buffer. The result was read after 30 minutes.

### Illustrative Assays

In one embodiment, the present application provides antigen-specific dIgA as a superior biomarker of recently acquired mucosal viral infections such as SARS-COV-2 infection, deployed in a rapid point of care lateral flow assay and in an ELISA or particle based format for screening subjects. The biomarker is superior to IgM for the detection of recent infection. Reference herein to recent infection is not the same as an acute or current infection because the presence of dIgA is shown herein to be indicative of a mucosal immune response which starts after infection begins and ends after viral levels have declined below a detectable level. Furthermore, the present screens are useful to determining the magnitude of the immune response to either infection or vaccination. In one embodiment, the magnitude of the dIgA immune response is determined by integrating the area under the curve (time vs dIgA levels). The area under the curve is useful for example because two subjects with equivalent levels of dIgA at a certain time after infection/vaccination/challenge (e.g., 11 days when they will all be positive after coronavirus infection) will have very different outcomes depending on whether this declines soon after, or persists for 2 months before declining.

From the plasma dIgA half-life data it is proposed herein that once B-cells leave the circulation, the dIgA follows soon after-therefore a more persistent high level dIgA response suggests ongoing production of new plasma B-cells to replace those that are going to the mucosa. This is clearly different to someone else who displays a short burst of plasma dIgA, wherein that “bolus” of cells goes to the mucosa and no more are produced-hence less B cells end up in the mucosa. Thus, for any pathogen, the trajectory of the dIgA response can be mapped to permit estimation of the full dIgA response of a subject. In another embodiment, screening at a two or more time points permit the construction of an individual curve.

Thus, screens according to the present invention are useful as dIgA only screens (other immunoglobulins may be screened for as controls or to provide additional information as described herein) or a combined dIgA and antigen/molecular screen for recent infection useful in providing further improvements in, for example, contact tracing.

In one embodiment, the lateral flow assay is based on the ability of a chimeric (rabbit/human) form of the polymeric Ig receptor, CSC, to bind with high specificity to dimeric IgA(7), thereby immobilising complexes of antigen-specific dIgA and SARS-COV-2 RBD antigen-colloidal gold. The ELISA assay complexes dIgA in plasma with CSC and anti-SC monoclonal antibody. The mixture is then applied to ELISA plates coated with SARS-COV-2 receptor binding domain antigen. Bound dimeric IgA-CSC-anti-SC complexes are detected with anti-mouse horse radish peroxidase conjugated antibody and TMD substrate.

The ability of CSC to bind dimeric IgA exclusively was confirmed using purified dimeric and monomeric IgA specific to the SARS-COV-2 RBD, with no reactivity observed towards monomeric IgA or IgG forms of the same immunoglobulin when present in ≥200-fold excess of the limit of detection for dIgA. Application of this assay to a large panel of pre-COVID plasma and a panel of samples from confirmed non-COVID-19 coronavirus infection showed assay specificity of 98.5%. The prototype assay can be performed on 15 μl of plasma or 30 μl of whole blood in under 30 minutes and provides a rapid assessment of serological activity towards SARS-COV-2.

Statistical methods—Data was analysed in Prism v9.02. Normality was determined using the D'Agostino & Pearson test. Data were correlated using the nonparametric Spearman test, two-tailed p value with 95% confidence intervals. A simple linear regression was used to calculate lines of best fit and r2. To fit appearance of IgG/A/M over time, a beta growth then decay curve was fitted to the data. To determine the half-life of dIgA, a one-phase decay equation was used with no constraints.
Purification of monoclonal antibodies—The monoclonal IgA antibodies were purified using Protein L agarose chromatography while IgG versions were purified using Protein G agarose (InvivoGen. Cat Code: gel-protl-2). Tissue culture supernatant from transfected cells was diluted 1:2 with equilibration buffer (10 mM Sodium Phosphate, 150 mM Sodium Chloride, pH7.2) so that 50 mls of SNF was added to 50 mls of equilibration buffer. 0.5 mls of immobilized Protein L Agarose was added and allowed to batch-bind for 2 hours at 4° C. The slurry was then added to a sintered glass chromatography column and the unbound material removed via gravity flow. The gel bed was washed with 20 mls of equilibration buffer. Antibodies were eluted with 10 ml of elution buffer (0.1 M Glycine, pH 2.7) and 1 ml fractions collected. The pH of the eluate was immediately adjusted to pH 7.5 by adding neutralization buffer (1M Tris pH 8.5). Protein content was assessed in each fraction using Bradford reagent (BioRad) and fractions containing the highest amount of IgA pooled. Purified immunoglobulins were buffer exchanged into PBS using Amicon 30K MWCO and the protein content measured by determining the OD280 nm using an extinction coefficient of 1.4. For purification of IgG, Protein G agarose replaced Protein L agarose.
Vectors Plasmids for the production of SARS-COV-2 retroviral pseudotyped particles, pHR′ CMV Luc, pCMV ΔR8.2, TMPRSS2 and WH-Human1_EPI_402119, were a kind gift from the NIH Vaccine Research centre. Plasmds encoding codon-optimsed full length spike proteins from the Beta (B.1.351) and Delta (B.1.617) variant were synthesized at Geneart and cloned into the WH-Human1_EPI_402119 vector to replace the ancestral Hu-1 sequence.
Pseudotyped virus production—Plasmid DNA from vectors pHR′ CMV Luc, pCMV ΔR8.2, TMPRSS2 and WH-Human1_EPI_402119 were purified (Qiagen Maxiprep) and transfected using Fugene6 transfection reagent according to the manufacturer's instructions into HEK293T cells. After 18 h incubation, media was removed and replaced with fresh DMF10 and cultures maintained for a further 48 h. Clarified supernatants containing retroviral pseudotyped viruses were filtered through a 0.45 uM filter and stored at −80° C. until use.

### Illustrative Antigen Assay or Kit Device

In one example, a complex is allowed to form between SARS-COV-2 nucleoprotein from within a blood sample and an immunoglobulin that specifically recognises the antigen, and the presence or absence of the complex is detected. A first antibody to nucleoprotein antigen is applied to a solid or semi solid surface such as a microwell plate, chromatographic strip, channel or bead. A recombinant CoV nucleoprotein antigen may be produced from an expression vector or purchased for example from a manufacturer to serve as a positive control. The optimal concentration of the antigen and serum dilutions are chosen after establishment titration assays using recombinant nucleoprotein diluted in normal (non-infected patient) plasma or serum. In a traditional ELISA assay for example, anti-nucleoprotein antibodies (or other specific antibody, antibody derivative or non-antibody binding agents (e.g., dIgA) may be applied to microwell plates blocked with Tween-20/skim milk, at a concentration of 1 μg/mL antibody diluted in 0.05 M carbonate-bicarbonate buffer and incubated in a humid chamber at 4° C. overnight. After each incubation step, the plates are rinsed with phosphate-buffered saline containing 0.05% Tween 20. Microtiter wells may be blocked with PBS-Tween supplemented with 5% skim milk by a 2 h preincubation at room temperature. The serum samples, diluted 1:200 in PBS-T supplemented with 2% skim milk, may be added to the wells and incubated for 1 h at 37° C. Following the rinsing steps, the wells may be incubated with a horseradish peroxidase-conjugated second anti-nucleoprotein antibody diluted 1:20,000 in the same buffer solution. The chromogenic solution TMB and substrate may be used for detection. The optical density (OD) in individual plate wells may be measured at 450/650 nm in an automatic ELISA reader. The cut-off value for a positive reaction is operationally defined as the mean OD plus three standard deviations as determined by testing serum samples from healthy pre-pandemic blood donors in the absence of added recombinant nucleoprotein antigen. Standard negative and positive controls as well as a threshold control (with OD equal to the cut off value) are analyzed in each ELISA run. The results are expressed as Reactivity Index (RI) by dividing the OD of each sample by the OD of the threshold control. The results may be considered positive if the RI value is ≥1.

Other suitable assays/kits known in the art include methods used in ELISA assays, chemiluminescence assays, indirect fluorescence, luciferase immunoprecipitation. Lateral flow assay formats are also numerous and include colloidal gold and fluorescence labelled immunochromatography.

It will be appreciated that the illustrative assay for detection of nucleocapsid antigen (or another antigen associated with a pathogen entering the body via mucosal surfaces) in plasma could be combined with the herein described assay for detection of antigen specific dIgA or anti-RBD dIgA antibody in plasma, in the same way that many assays for infection with human immunodeficiency virus (HIV) combine the detection of HIV p24 antigen together with the detection of antibody to antigens other than p24 such as gp41, gag etc.

dIgA assays described herein allow for positive detection of elevated dIgA levels over a period of up to 100 days. Using combined tests pf dIgA and molecular or antigen tests, permits expanded detection of a recent infection to an earlier time point. For example, both are positive, then antigen+dIgA testing (via laboratory or rapid strip/kit/device testing) could replace the PCR based tests. If people are symptomatic, or close contacts of symptomatic people (past or current) then by inference the window is narrower for defining recent. If asymptomatic then the window is wider if you return an Antigen negative test and dIgA positive test.

In one embodiment, LFA may employ CSC-linked anti-NP dIgA in the sample pad/mobile phase, to capture NP (taking advantage of dIgA avidity), and then capture these complexes with an antibody specific for CSC e.g., a monoclonal antibody to the rabbit domain 1. In one embodiment, a CSC conjugate on gold, mixed with a dIgA anti-NP antibody, is used as the detection reagent for NP, which would stop the NP dIgA from reacting with the CSC test line.

In another embodiment, as NP is a dimer in solution it could be crosslinked to form larger aggregates with higher avidity binding to whatever test line/detector is used. Thus, in one embodiment, IgM might be especially useful for achieving crosslinking, and the use of CSC for dIgA would avoid any interference between the two reactions.

The reliable temporal pattern of dIgA and the sensitivity of pIgR based reagents, makes dIgA and dIgA-pIgR complex detection better than other antibody isotypes and assays for early detection of recent infection with contagious respiratory viral infections such as CoV.

A Cochrane review, Deeks, J. J. et al. Antibody tests for identification of current and past infection with SARS-COV-2. Cochrane Database Syst Rev 6, CD013652 (2020) disclose the standard approach to diagnosis of COVID-19 is through a reverse transcription polymerase chain reaction (RT-PCR) test, which detects the presence of virus in swab samples taken from nose, throat or fluid from the lungs. However, the test is known to give false negative results, and can only detect COVID-19 in the acute phase of the illness. Both the World Health Organization (WHO) and the China CDC (National Health Commission of the People's Republic of China), have produced case definitions for COVID-19 that include RT-PCR-negative cases that display other convincing clinical evidence. The most recent case definition from the China CDC includes positive serology tests. Confirming an acute clinical diagnosis using a serology test requires detectable virus-specific IgM and IgG in serum, or detectable virus specific IgG, or a 4-fold or greater increase in titration to be observed during convalescence compared with the acute phase.

The review of 57 serological tests of 54 cohorts showed that the sensitivity at day 8-14 for IgG was 57.9-74.2% and for IgM was 45.5-70.3% with average specificity of 99.1% and 98.7%, respectively (Deeks, J. J. et al. Cochrane Database Syst Rev 6, CD013652 (2020). As such, the currently available CLIA (Clinical Laboratory Improvement amendments) certified serological assays are considered unreliable for diagnosis of early or recent infection.

Rapid contact tracing has emerged as one of the pivotal public health measures to limit viral spread in the community and within health care settings. A significant gap in diagnostic testing remains for scenarios where backward contact tracing is required to identify primary source cases and their contacts who may now be SARS-COV-2 antigen and/or RNA negative but have had recent infection, especially in healthcare settings, assisted living facilities and nursing homes, schools and high-risk workplaces.

Serological approaches can be useful for backward contact tracing but this is reliant on the temporal pattern of assay sensitivity, both in the appearance of the particular antibody reactivity and in its disappearance after a predictable length of time. The low sensitivity of these serological tests, especially early in infection (<11 days), limits their use in situations where every contact must be identified as soon as possible to track and trace recent transmission.

All currently available tests for SARS-COV-2 specific IgA measure total IgA, with 98.7% sensitivity (39.0-100) at day 15-21 and only 78.1% sensitivity (9.5-99.2) at day 8-14 Watson, J., Richter, A. & Deeks, J. Testing for SARS-COV-2 antibodies. BMJ 370, m3325 (2020). There is limited information on the longevity of these IgA responses, complicated by the highly variable sensitivity of IgA assays, but in a sensitive assay for spike-specific IgA in longitudinal sera the half-life was estimated to be 210 days (126-627 days) (Dan, J. M. et al. Immunological memory to SARS-COV-2 assessed for up to 8 months after infection. Science 371 (2021)), which precludes the identification of recent infections compared to more distant past infections.

The inventors have developed a novel point of care assay, lateral flow assay (LFA) that specifically detects dimeric IgA against SARS-COV-2 antigen, e.g., a receptor binding domain (RBD) of the SARS-COV-2 spike protein in plasma and examined the temporal production of dIgA and its longevity in plasma post SARS-COV-2 infection.

### EXAMPLES

### Example 1—Materials

### Recombinant Protein L

Supplier: ThermoFisher Scientific
Cat #: 21189
Concentration: 25 ug/mL

### Mouse Anti-human IgA

Supplier: Sigma
Cat #: 10636
Concentration: 0.5 mg/ml
   Mouse anti Human IgA2 (subclass specific)
Supplier: Nordic Mubio
Cat #: MAHu/IgA2
Concentration: 0.25 mg/mL
   CSC (Chimeric Secretory Component, chimeric polymeric Ig receptor)
Supplier: In-house (Burnet Institute as described in WO/2014/071456), expressed in mammalian cells
Recombinant His Tag expressed in 293T, and gel filtration purified. >95% purity (Dimer)
Concentration: 1 mg/mL

### Streptavidin 40 nm Gold Conjugate

Supplier: Abcam
Cat #: ab186864
Concentration: OD10
   Mouse Anti-human secretory component clone 6D5
Burnet/Boint Biotech

### Anti-Mouse IgG HRP

Abcam

### Burnet RBD (SARS-COV-2)

Expressed in 293Expi cells, purified via a poly-Histidine Tag and subjected to size-exclusion chromatography. >95% purity.

### Burnet RBD-biotin

Expressed in Freestyle 293F cells stably transfected with BiRA ligase, purified via a poly-Histidine Tag and subjected to size-exclusion chromatography. >95% purity.
   Acro Biosystems S protein
Cat #: SPN-C52H4 Lot No. 3534b-204F1-R9
Source: SARS-COV-2 S protein (R683A, R685A) expressed in HEK293 cells. His tag at C terminus.
Formulation: Lyophilized from 425 ul bulk protein in a 0.2 μm filtered solution of 50 mM Tris, 150 mM NaCl, pH7.5, with 10% trehalose as protectant.
   EastCoast Bio N protein
Cat #: LA600
Source: SARS-COV-2 N protein (aa 2-419) expressed in E. coli cells. His tag purified.
Formulation: 25 mM Tris, 10 mM Potassium Carbonate, pH 7.4.
   EastCoast Bio monoclonal antibody to N protein clone HM1056 (herein EB56)
Cat #: HM1056
Source: SARS-COV-2 N, Recombinant Coronavirus nucleoprotein peptide, immunisation of mice.
Formulation: Phosphate buffered saline, 1 mg/ml.
   Blocking solution
Abacus Protein Stabilizer Cat #: 5D-82411B
Liquid, sterile, highly purified 10% hydrolyzed porcine collagen peptide fraction. Manufactured by 5-Diagnostics, available in bulk.

### Running Buffer

Plasma: PBS+0.5% Tween+0.05% Azide Whole blood: 1% Triton X-100 in PBS
   Drying conjugate Buffer (BDS, used for dilution of the gold conjugate for drying)
20% Sucrose, 5% Trehalose, 0.25% Tween 20, 0.35% PEG, 1% BSA, 2 mM EDTA, 10 mM Borate
   SDS-PAGE-SARS-COV-2 S protein (R683A, R685A) on SDS-PAGE under reducing conditions. The gel was stained overnight with massive Blue. The purity of the protein is great than 85%. Patient Samples were obtained from patients in various cohorts of
PCR confirmed COVID-19+ve individuals
Community sampling, mild and asymptomatic, cross-sectional n=60
Hospitalised patients n=10 (22 timepoints)

TABLE 1
Biobank patient characteristics

| Age (Mean, | 40, 35 years |
|---|---|
| Median) | |
| Male | 33/60 (55%) |
| Female | 27/60 (45%) |
| Days from Dx: | |
| ≤55 days | 12/60 (20%) |
| >55 days | 48/60 (80%) |

### Example 2—Lateral Flow dIgA and Total Antibody Point of Care Test Device

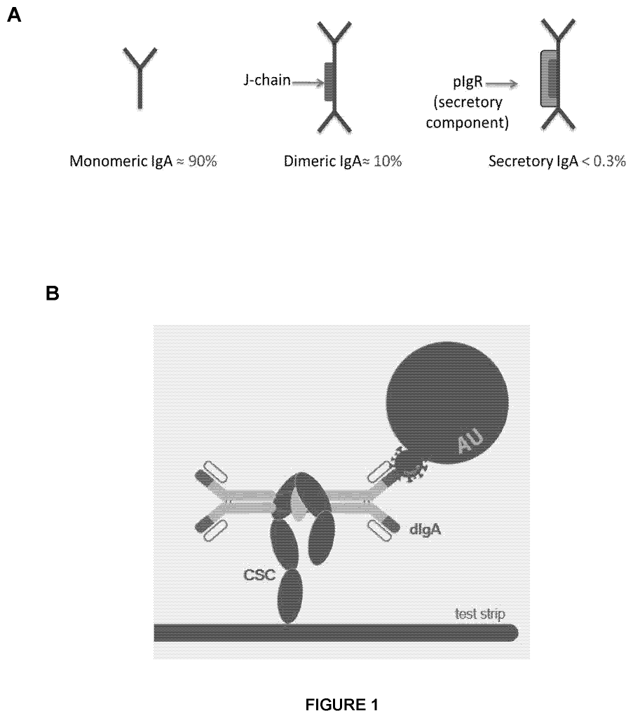

During Step 1 and at the start of Step 2, the sample plasma and buffer rehydrate the colloidal gold streptavidin-RBD biotin conjugate (RBD-gold) that is dried in the sample pad of the prepared device. Antibodies to RBD, including but not limited to dIgA antibodies, bind to the RBD and consequently to the colloidal gold. With the addition of more buffer during Step 2, the mixture of plasma, buffer, RBD-gold and any patient antibodies bound to RBD-gold migrates along the lateral flow strip. In the order of sample flow, a substantial proportion of patient dIgA is captured at T2 (CSC test line) due to the interaction of dIgA with CSC. If any of the dIgA is specific to RBD, then RBD-gold will also be immobilised at this test line (as shown in FIG. 1), whereas if there is no dIgA present with specificity to RBD, then the line will be clear with no visible signal. After further flow along the strip, the mixture of plasma, buffer and RBD-gold (including RBD-gold with bound RBD-specific antibodies) interacts with the T1 (RBD test line), and antibodies of any isotype that are specific to RBD will bind to the T1 test line. Because antibodies of humans and most animals have at least 2 binding sites for antigen (Fab domains) in the example of IgG or monomeric IgA, or 4 binding sites in the example of dIgA, or 10 binding sites in the example of pentameric IgM, a proportion of the RBD-specific antibodies that bind to the RBD test line via a first Fab domain will have already bound to RBD-gold via a second or subsequent Fab domain, resulting in RBD-gold being immobilised at this test line. Samples may be positive for RBD-specific dIgA alone, which is likely at the very earliest stage after infection, or dIgA and total antibody, which is likely up to 2-3 months after infection, or total antibody alone which is likely more than 3 months after infection. The relative amount of dIgA and total antibody may also indicate time after infection, such that samples may have low or moderate levels of dIgA together with similar levels of total antibody very early in infection (less than around 12 days after symptom onset), high levels of both dIgA and total antibody which is likely up to 1-2 months after infection, or low or moderate levels of dIgA together with relatively high levels of total antibody which is likely more than 2-3 months after infection. The absence of both dIgA and total antibody suggests that the individual has not been infected with SARS-COV-2, or that any antibody from infection or immunisation has waned to undetectable levels. While dIgA is expected to also be reactive for the total antibody test line, it is substantially depleted from the sample after flowing over the CSC test line and may no longer be detectable or be detectable at much lower levels at the total antibody test line after such depletion.

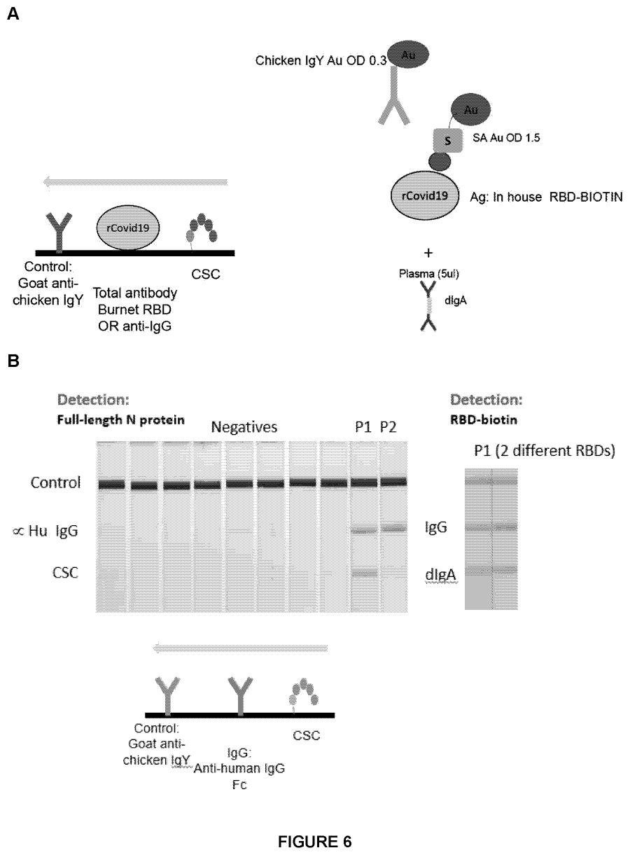

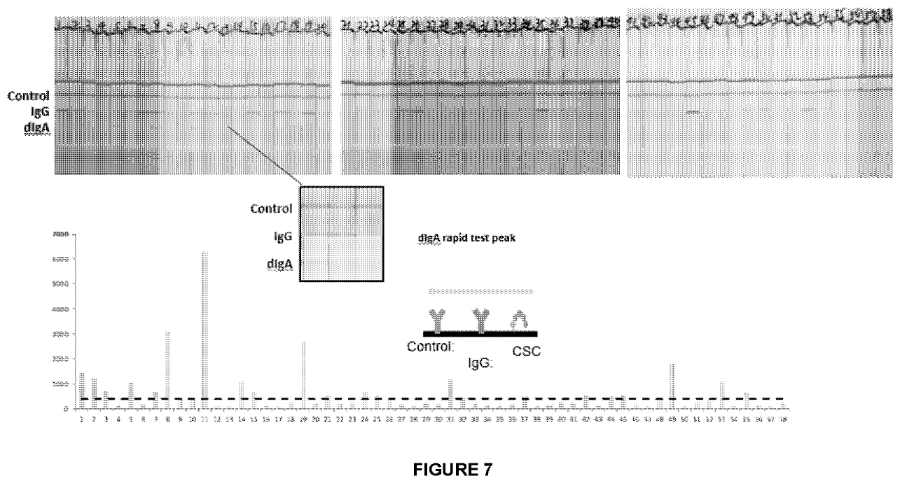

The same principles can be used for detection of dIgA specific to other antigens such as N protein from SARS-COV-2 (or antigens such as those comprising the RBD from other pathogens, i.e., neutralizing or non-neutralizing antigens), and antigen-specific IgG can be detected instead of total antibody. Both of these approaches are shown in FIG. 6A, where the T1 test line can be either anti-IgG antibody OR the cognate antigen that is linked to visible marker such as gold, and the antigen can be either RBD (in this case RBD-biotin conjugated to streptavidin gold) or N protein (in this case N protein bound to gold that has been conjugated with monoclonal antibody EB56 (EastCoast Bio) via passive absorption. FIG. 6B shows an example of lateral flow tests detecting dIgA and IgG antibody to N protein (left hand panel) or RBD protein (right hand panel). As determined herein, surprisingly, IgM levels over time provide no information regarding the timing of infection or the magnitude of the immune response, showing an R² of 0.0119 compared to dIgA which showed a strong correlation/relationship (R²=0.3903). Thus in one embodiment, the level of IgM is not determined.

FIG. 7 shows an expanded panel of samples from patients with PCR-confirmed SARS-COV-2 infection tested for dIgA and IgG antibody specific for RBD using the format of anti-IgG (T1 test line) and CSC (T2 test line). The upper panel shows a photographic representation of the test results, and the lower panel shows a graphical representation of the test results for the dIgA (T2 test line) only, after reading of the tests in the Axxin AX-2XS instrument, where the cutoff for visual detection is around 400 units (shown with a dashed line).

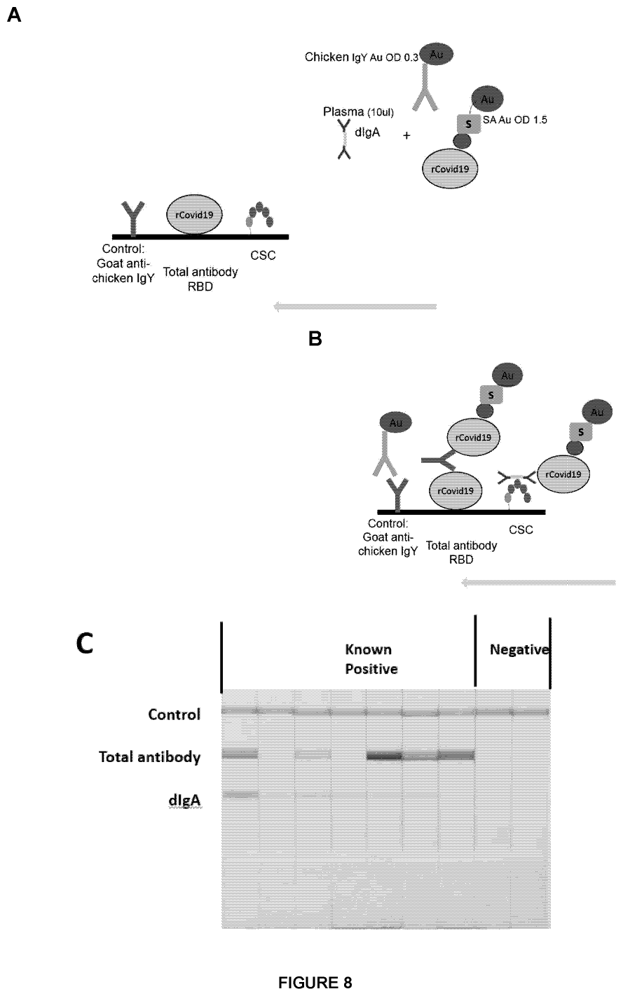

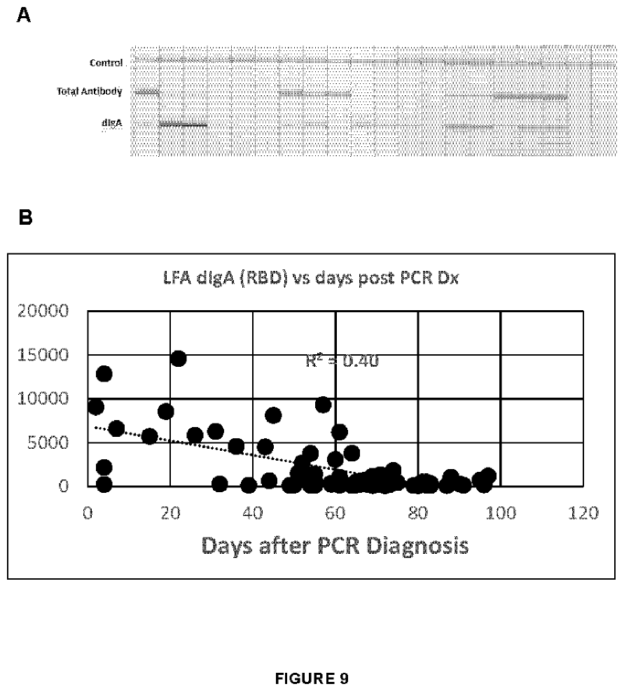

FIG. 8 shows a schematic of the format of one preferred embodiment of the test in more detail, where T1 test line is RBD antigen (measuring total antibody) and T2 test line is CSC (measuring dIgA). In panel A, the different components of the test “sample pad” are shown, with the Chicken IgY-conjugated gold and RBD-biotin streptavidin gold conjugates being pre-dried in the test strip, and rehydrated on addition of plasma and buffer, while CSC (T2 test line), RBD (T1 test line) and anti-chicken IgY (control line) are immobilised on the lateral flow nitrocellulose membrane. Panel B shows a schematic example of the complexes that are formed when both dIgA and IgG (or other isotypes) specific to RBD are present, resulting in linkage of both dIgA and IgG to the RBD-biotin-streptavidin-gold conjugate and subsequent immobilisation of the visible marker (gold) at the T1 and T2 test lines, while the Chicken IgY gold conjugate is immobilised at the anti-chicken IgY control line. Panel C shows examples of results for samples known to be from patients with SARS-COV-2 infection (known positive) or health controls (negative), with a range of reactivities in positive samples for dIgA and total antibody, while the control line is always present.

FIG. 9 shows further examples using the same test format. Panel A shows representative samples from patients with PCR-confirmed SARS-COV-2 infection where plasma samples were collected at various times after PCR diagnosis. Panel B shows a graphical representation of the dIgA (T2 test line) intensity only, versus days after PCR diagnosis. There is a strong correlation with decreasing dIgA line intensity against increasing time after diagnosis with a Spearman coefficient (R2) of 0.40.

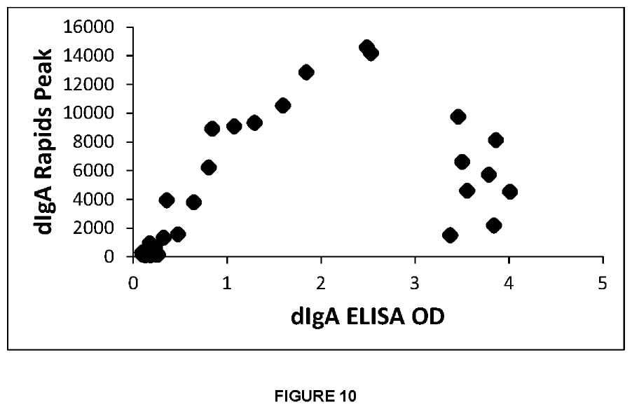

FIG. 10 shows the correlation between dIgA reactivity observed using either the dIgA ELISA or the dIgA lateral flow test for RBD-specific antibodies. It is evident that there is very close correlation and agreement for the level of dIgA reactivity by both methods when the ELISA OD is less than around 3.0, but with higher levels of dIgA reactivity there is evidence of a “prozone” effect with reduced levels of dIgA detected in the lateral flow assay. As the dIgA is increasing (along with total antibody) there is no prozone effect, but when the total antibody reaches higher levels it reaches a prozone and part of the rapid decline observed in the lateral flow assay is due to increasing competition with total antibody, as well as genuine reduced levels of dIgA. Whilst this effect may in some embodiments be useful epidemiologically, it is also an effect that can be corrected for.

FIG. 11, Panel A shows a graphical representation of the dIgA (T2 test line) intensity only, versus days after PCR diagnosis, for the same samples shown in FIG. 9 but after arithmentic correction for the “prozone” effect described in FIG. 10. For the samples with ELISA OD of >3, samples were assigned the maximum observed Axxin test line intensity of 15,000. After such correction there is an even stronger correlation with decreasing dIgA line intensity against increasing time after diagnosis with a Spearman coefficient (R²) of 0.51. Again there is a trend towards rapid loss of detectable dIgA antibody at more than around 75 days after PCR diagnosis.

FIG. 11, Panel B shows a graphical representation of both the ELISA (bars) and lateral flow Axxin readout (lines) for a single patient with samples collected at 7 day intervals from 57 to 71 days after PCR diagnosis, demonstrating this rapid decline of specific dIgA over time. Taken together, these examples show that antigen-specific dIgA is a reliable marker of recent infection, in this case RBD-specific dIgA as a marker of recent SARS-COV-2 infection.

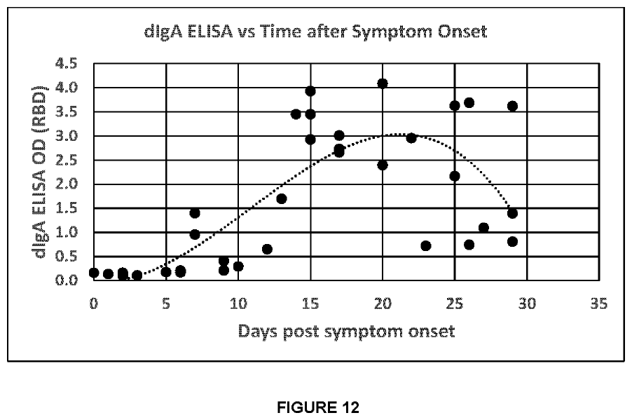

FIG. 12 shows a graphical representation of results for RBD-specific dIgA ELISA for a separate cohort of SARS-COV-2 patients where plasma samples were collected at various days after the first onset of symptoms, rather than days after PCR diagnosis. Samples collected in the first 5 days after symptom onset do not show reactivity, reflecting the time required for a patient to elicit an antibody response after symptoms but in many cases before presenting for diagnosis. Some patients are seen to have detectable RBD-specific dIgA as early as 7 days after first symptoms in this example, and all patients are positive for samples between 12 and 29 days after symptom onset.

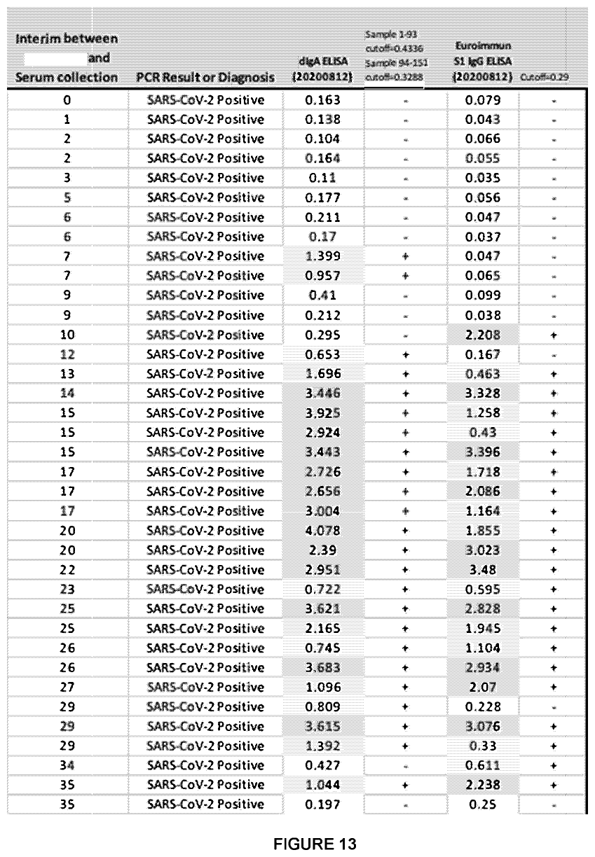

FIG. 13 shows a comparison between the ELISA ODs for RBD-specific dIgA shown in FIG. 12, and a commercial ELISA for S1-specific IgG (EuroImmun) performed using the same samples and manufacturers instructions, showing that 2/21 samples had undetectable IgG but detectable dIgA between 12 and 29 days after symptom onset.

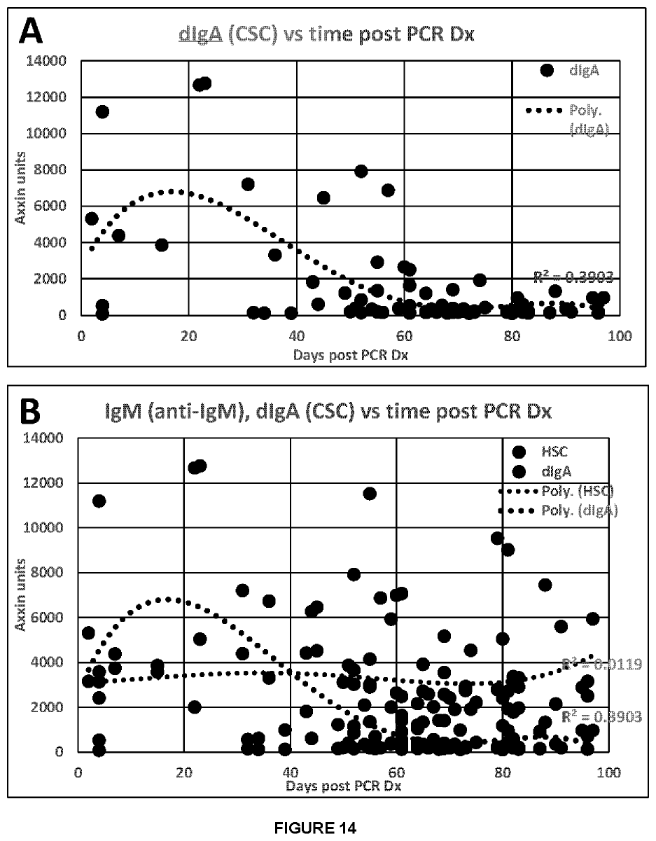

IgM-class antibodies are commonly used as a marker of recent or acute infections, but they tend to exhibit cross-reactivity. In the case of SARS-COV-2, it is not known how well they correlate with time after infection. To examine this question, the presence of RBD-specific IgM was detected using a lateral flow assay with essentially the same format and reagents as shown in FIG. 8 for RBD-specific dIgA, but with anti-human IgM antibody replacing CSC as the T2 test line, so that T2 binds IgM instead of dIgA. FIG. 14 shows the results for samples from patients with PCR-confirmed SARS-COV-2 infection at various times after PCR diagnosis tested for dIgA (red, same data shown in Panel A and Panel B) or for IgM (blue, Panel B only). Surprisingly, the level of IgM reactivity shows no correlation with time after PCR diagnosis, at least in the period to around 90 days after diagnosis, in contrast to the level of dIgA reactivity. As shown previously, the native human pIgR can be used to bind both IgM and dIgA, and similar results are seen using human pIgR (HSC) instead of anti-human IgM in these experiments. Because there is a large excess of total IgM versus total dIgA present in plasma, it is likely that the HSC (human pIgR) reagent binds relatively little dIgA compared to CSC where only the minor fraction representing dIgA is able to bind.

### Example 3-Measurement of Neutralizing Antibodies in Plasma from a Convalescent COVID-19 Subject

Plasma was obtained from an individual at various time points (time point 1 to 5, every 2 to 4 weeks) after SARS-COV-2 infection and heat inactivated at 56° C. for 30 minutes. Plasma was serially diluted and mixed in triplicates with an equal volume of SARS-COV-2 (ancestral Hu-1 spike) retroviral pseudotyped virus and incubated for 1 h at 37° C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The reciprocal dilution of plasma required to prevent 50% virus entry was calculated from the non-linear regression line (ID50). The negative sample is plasma obtained pre-December 2019. The lowest amount of neutralizing antibody detectable is a titre of 20. All samples that did not reach 50% neutralization were assigned an arbitrary value of 10.

The example illustrated in FIG. 15 shows that neutralizing antibodies can be detected in convalescent plasma obtained from a person with confirmed SARS-COV-2 infection, but not in a sample of plasma obtained from a different individual prior to December 2019 who did not have SARS-COV-2 infection (pre-COVID-19). The strength of neutralization wanes over time, with the highest titre of neutralizing antibody detected in sample 1, earliest time point after infection, and the lowest titre detected in sample 5, the longest time point after confirmed SARS-COV-2 infection.

### Example 4—Neutralizing Antibody Activity in Biobank Plasma Samples from Convalescent COVID-19 Subjects

Plasma was obtained from individuals at various time points after SARS-COV-2 infection and heat inactivated at 56° C. for 30 minutes. Plasma was serially diluted and mixed in triplicates with an equal volume of SARS-COV-2 (ancestral Hu-1 spike) retroviral pseudotyped virus and incubated for 1 h at 37° C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The negative sample is plasma obtained pre-December 2019. The reciprocal dilution of plasma required to prevent 50% virus entry was calculated from the non-linear regression line (ID50). The lowest amount of neutralizing antibody detectable is a titre of 20. All samples that did not reach 50% neutralization were arbitrally assigned a value of 10. The data in FIG. 16 show the ID50 titres of 58 convalescent plasma samples obtained from a Biobank of COVID-19 subjects. It can be seen that the amount of neutralizing antibodies is variable between subjects.

### Example 5—Longitudinal Analysis of Neutralizing Antibodies in Plasma Samples from Convalescent COVID-19 Subjects

Neutralizing antibody responses were determined after confirmed PCR diagnosis. Plasma was obtained from an individual (Patient 1) at various time points (time point 1 to 5, every 2 to 4 weeks) after SARS-COV-2 infection and heat inactivated at 56° C. for 30 minutes. Plasma was serially diluted and mixed in triplicates with an equal volume of SARS-COV-2 (ancestral Hu-1 spike) retroviral pseudotyped virus and incubated for 1 h at 37*C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The reciprocal dilution of plasma required to prevent 50% virus entry was calculated from the non-linear regression line (ID50). The negative sample is plasma obtained pre-December 2019. The lowest amount of neutralizing antibody detectable is a titre of 20. All samples that did not reach 50% neutralization were arbitrally assigned a value of 10.

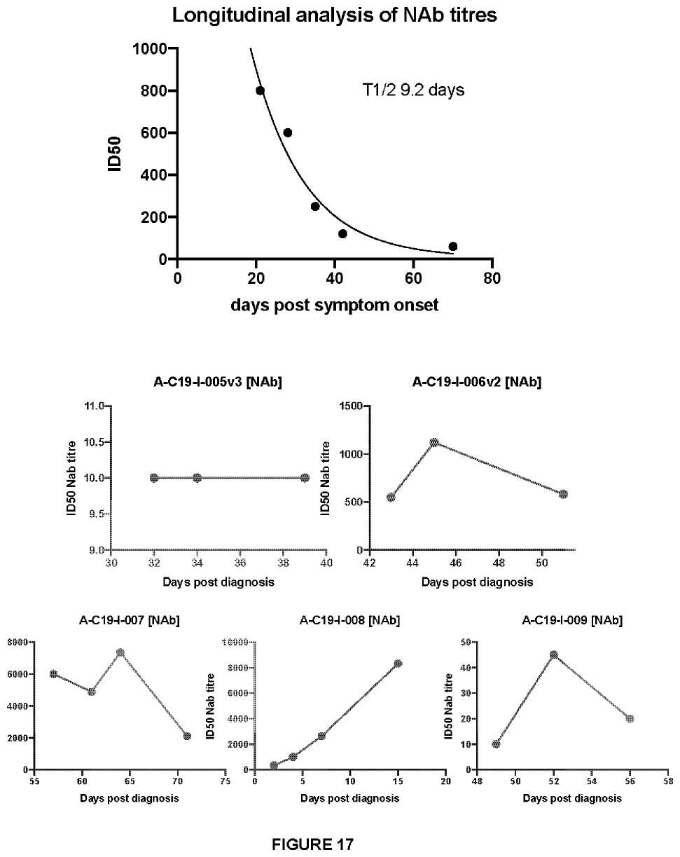

FIG. 17 shows that people infected with SARS-COV-2 have varying titres of neutralizing antibodies detectable in the plasma and varying trajectories. In Patient 1, the half life of neutralizing antibody titres is calculated to be 9.5 days. In subject 005v3, no neutralizing antibodies were detectable at the highest concentration of plasma used, while in subject 008, neutralizing antibodies were steadily increasing post PCR diagnosis. In subjects 006v2, 007 and 009, neutralizing antibodies titres were declining in the last time point analysed.

### Example 6—Stratification of Convalescent COVID-19 Subjects Based on Levels of Neutralizing Antibodies and Other Measured Parameters

Neutralizing antibody responses after confirmed PCR diagnosis. Plasma was obtained from patients at various time points after SARS-COV-2 infection and heat inactivated at 56° C. for 30 minutes. Plasma was serially diluted and mixed in triplicates with an equal volume of SARS-COV-2 (ancestral Hu-1 spike) retroviral pseudotyped virus and incubated for 1 h at 37*C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The reciprocal dilution of plasma required to prevent 50% virus entry was calculated from the non-linear regression line (ID50). The negative sample is plasma obtained pre-December 2019. The lowest amount of neutralizing antibody detectable is a titre of 20. All samples that did not reach 50% neutralization were arbitrally assigned a value of 10.

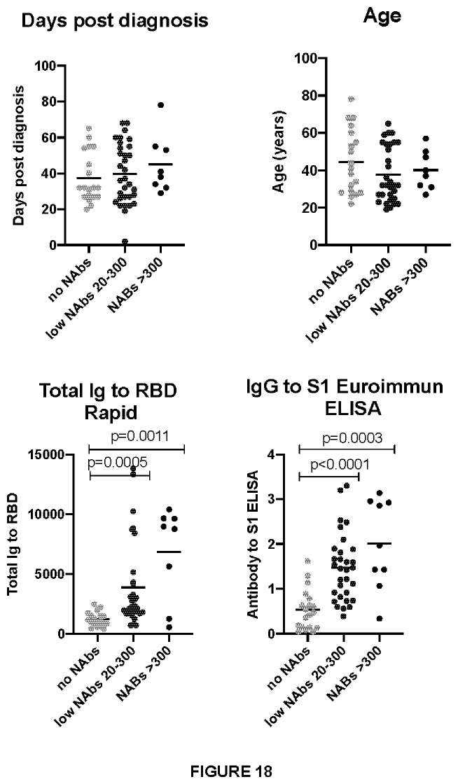

FIGS. 18A) and B) shows the levels of neutralizing antibodies detected in people according to days post diagnosis and age. The neutralizing antibody (NAb) levels of 58 subjects from a biobank of COVID-19 convalescent plasma were measured and then stratified into three groups. The “no NAbs” group had no detectable ability to prevent 50% inhibition of virus entry at the lowest dilution of plasma tested (1/20). The second group had ID50 titres of between 20 and less than 300. The third group had high NAB titres of >300. There was no difference in the age of subjects that stratified into these three groups, nor, the number of days post diagnosis (p>0.05). Data were analysed using a non parametric Kruskal-Wallis test with multiple comparisons.

FIG. 18 shows the levels of neutralizing antibodies detected in people according to the total immunoglobulin detected C) towards the SARS-COV-2 receptor binding domain (RBD) and D) specific immunoglobulin G to the S1 domain of SARS-COV2 using a Euroimmun test. The neutralizing antibody (NAb) levels of 58 subjects from a biobank of COVID-19 convalescent plasma were measured and then stratified into three groups. The “no NAbs” group had no detectable ability to prevent 50% inhibition of virus entry at the lowest dilution of plasma tested (1/20). The second group had ID50 titres of between 20 and less than 300. The third group had high NAB titres of >300. Data were analysed using a non parametric Kruskal-Wallis test with multiple comparisons. Subjects in the >300 NAb titre group had higher levels of total immunoglobulin towards the RBD (p=0.0011) and the S1 domain (p=0.0003) than subjects with no detectable NAb. In addition, subjects with NAb titres of between 20-300 had significantly higher titres of immunoglobulin to the RBD (p=0.0005) and the S1 domain (p<0.0001) than those with no detectable NAbs.

### Example 7—Stratification of Convalescent COVID-19 Subjects Based on Levels of Neutralizing Antibodies and IgA

Neutralizing antibody responses after confirmed PCR diagnosis. Plasma was obtained from patients at various time points after SARS-COV-2 infection and heat inactivated at 56° C. for 30 minutes. Plasma was serially diluted and mixed in triplicates with an equal volume of SARS-COV-2 (ancestral Hu-1 spike) retroviral pseudotyped virus and incubated for 1 h at 37*C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The negative sample is plasma obtained pre-December 2019. The lowest amount of neutralizing antibody detectable is a titre of 20. All samples that did not reach 50% neutralization were assigned an arbitrary value of 10. The reciprocal dilution of plasma required to prevent 50% virus entry was calculated from the non-linear regression line (ID50).

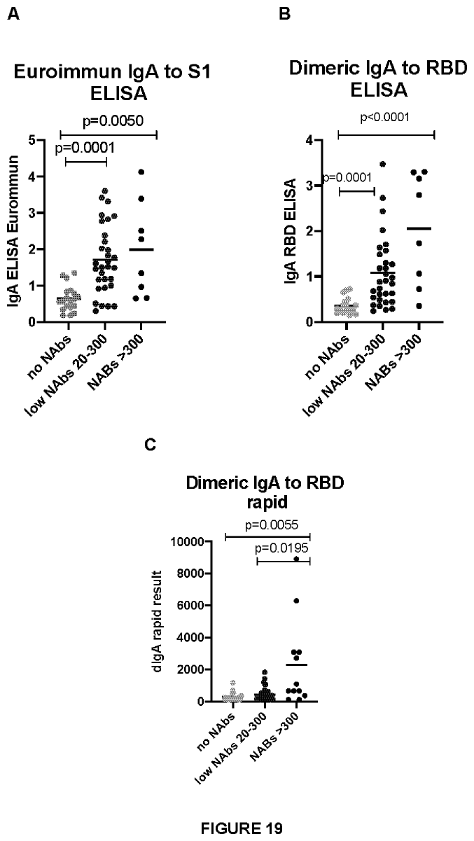

FIG. 19 shows a comparison of titres with levels of IgA (A) and dimeric IgA (B and C). The neutralizing antibody (NAb) levels of 58 subjects from a biobank of COVID-19 convalescent plasma were measured and then stratified into three groups. The “no NAbs” group had no detectable ability to prevent 50% inhibition of virus entry at the lowest dilution of plasma tested (1/20). The second group had ID50 titres of between 20 and less than 300. The third group had high NAb titres of >300. Data were analysed using a nonparametric Kruskal-Wallis test with multiple comparisons. Subjects with the highest tires of NAbs >300 had significantly higher levels of IgA towards the S1 domain (p=0.0050) (A), and towards the RBD as measured using ELISA (B) (p<0.0001) and as measured in a rapid point of care test (C) (p=0.0055) compared to subjects with no detectable levels of NAb. In addition, subjects with the tires of NAbs between 20-300 had significantly higher levels of IgA towards the S1 domain (A) (p=0.0001), and towards the RBD as measured using ELISA (B) (p=0.0001). Subjects with NAb titres >300 (C) had significantly higher levels of dimeric IgA as measured using a lateral flow rapid point of care test (p=0.0195) compared to subjects with NAb titres between 20-300.

### Example 8—Depletion of Serum dIgA in Plasma from Convalescent COVID-19 Subjects

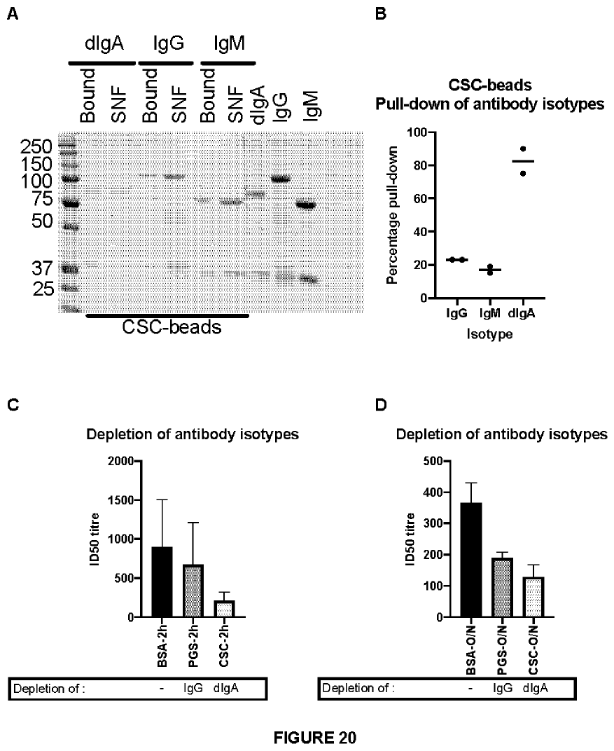

Chimeric secretory protein (CSC) beads were prepared by adding 10 mg purified CSC protein to 0.3 grams of dried NHS agarose beads in a 10 ml volume in PBS and rotated end-over-end for 3 hours at room temperature. The ability of CSC beads to capture IgA, IgG and IgM was assessed in an immunoprecipitation assay format. Antibody (3 ug) was added to 50 ul of the slurry containing 5 ul of beads. Purified dimeric IgA and IgM and IgG was added and rotated end-over-end, 4° C. for 2 hours. Beads were centrifuged 2,000×g, 2 minutes at room temperature and the supernatant fluid removed followed by two additional washes with 1 ml of PBS containing 0.05% Tween 20 and a further two washes with 1 ml of PBS with centrifuging 2,000×g, 2 minutes in between washes. The initial supernatant and washed beads in a 20 ul volume were then assessed for Ig content in SDS-PAGE under reducing conditions on 12% SDS/PAGE slab gels. The SDS-PAGE gel was stained with Coomassie blue and bands quantitated on an Odyssey imager. The results are shown in FIG. 20. FIG. 20A shows that CSC beads were able to preferentially bind dIgA, with a much lower binding efficiency towards IgG and IgM isotypes. FIG. 20B shows the quantitation of the SDS-PAGE and demonstrates that approximately 80-90% of the digG could be bound by CSC-beads whilst only approximately 20% of IgM and IgG was bound by CSC-beads.

Plasma from a convalescent subject with high titre NAb was added to CSC beads, protein G Sepharose beads (PGS) which preferentially binds IgG, or bovine serum albumin (negative control) protein coupled to sepharose (BSA) for either 2 hours or overnight. The residual neutralization activity in the plasma was measured by mixing in triplicates serial dilutions of plasma with an equal volume of SARS-COV-2 (ancestral Hu-1 spike) retroviral pseudotyped virus and incubated for 1 h at 37° C. Virus-plasma mixtures were added to 293T-ACE2 cell monolayers in 96 well plates seeded the day prior and incubated for 2 h at 37° C. before addition of an equal volume of tissue culture medium and incubated for 3 days. After 3 days, tissue culture fluid was removed, monolayers were washed once with PBS and lysed with cell culture lysis reagent (Promega) and luciferase measured using luciferase substrate (Promega) in a Clariostar. The mean percentage entry was calculated as (RLU plasma+virus)/(RLU medium+virus)*100. The percentage entry was plotted against the reciprocal dilution of plasma in Prism v8.3.1 and curves fitted with a one-site specific binding Hill plot. The reciprocal dilution of plasma required to prevent 50% virus entry was calculated from the non-linear regression line (ID50). The results are shown in FIG. 20. FIGS. 20C and 20D show that CSC-beads depleted the neutralization activity in the plasma after either 2 h or overnight incubation with convalescent plasma and reduced the ID50 titre approximately 70-80% compared to the BSA control depletion. Protein G Sepharose beads which do not bind IgA, instead binding IgG isotypes strongly, depleted approximately 25-50% of the neutralization activity in the plasma.

### Example 9—Assessment of dIgA (RBD) and Pseudoneutralization Titres for Longer Term Convalescent Samples

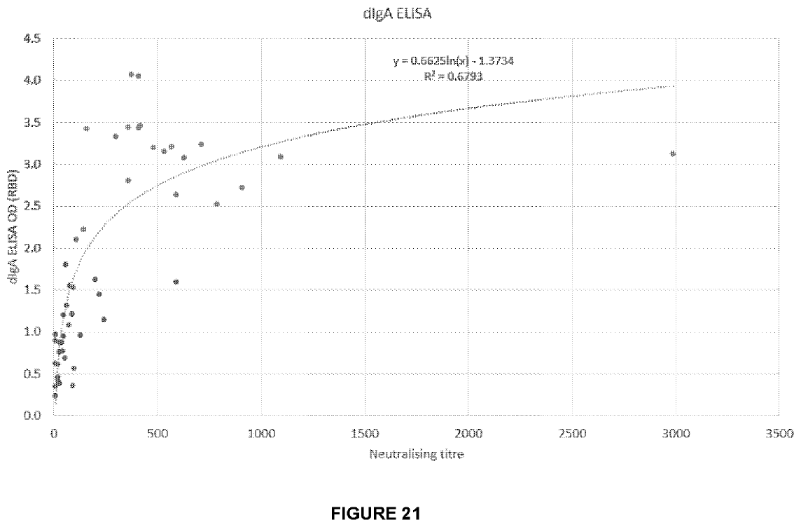

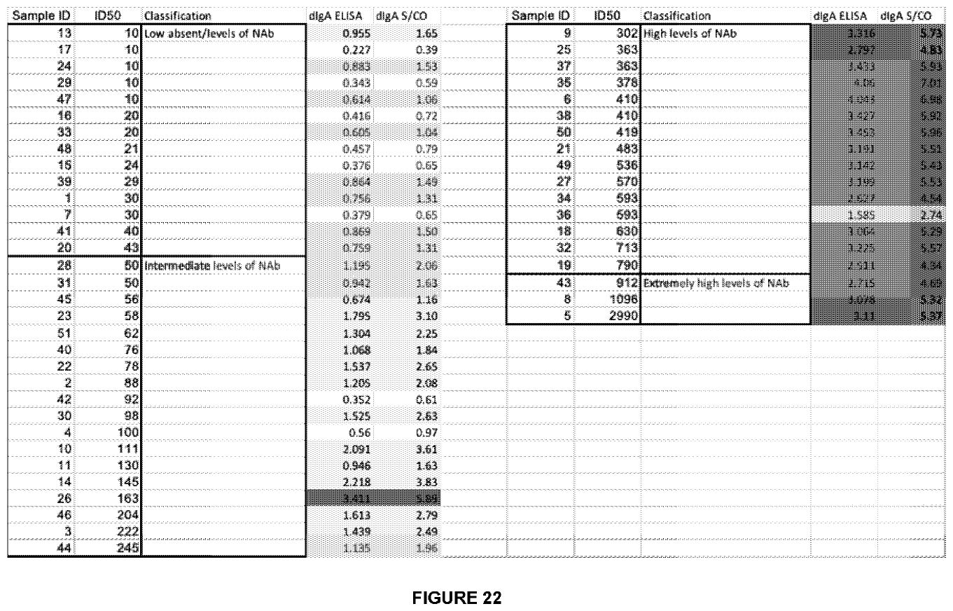

dIgA (RBD) and pseudoneutralization titres for longer term samples were assessed the results of which are presented in FIGS. 21 and 22.

Plasma samples from 6 patients with PCR-confirmed COVID-19 were collected from commercial plasma donors before infection (one sample per patient), and at weekly intervals for 8 or 9 weeks after patients became PCR negative (end of isolation phase). Plasma 50% endpoint Neutralizing antibody titres were measured by pseudovirus assays as before, and IC50 values were correlated with the level of RBD-specific dIgA by ELISA as before. A strong correlation between the total level of neutralizing antibody and the level of RBD-specific dIgA is seen (FIG. 21).

In addition to the strong arithmetic correlation seen in FIG. 21, the measurement of RBD-specific dIgA by the simple method of ELISA allowed the sensitive and specific characterization of samples highlighted in green with high levels of NAb (endpoint titre greater than 250) and extremely high levels (endpoint titre greater than 800), which usually had dIgA ELISA OD of greater than 1.5 or alternatively expressed as a sample/cutoff (S/CO) ratio of 2.6 by comparison to a cutoff control for dIgA in the same assay (FIG. 22). Samples with negligible levels of NAb (endpoint titre less than 50, which presumably includes the 6 samples collected prior to infection) or intermediate levels of NAb (endpoint titre 50-250) generally had RBD-specific dIgA ELISA OD of less than 1.5 or S/CO ratio of 2.6 (highlighted in yellow) or were negative for dIgA by ELISA (no highlighting).

### Example 10—an Embodiment of the Immune Response Surveillance Method: SARS-CoV-2 (COVID-19) Total Ig and dIgA Test

The SARS-COV-2 (COVID-19) total Ig and dIgA test is for the qualitative detection of novel coronavirus (SARS-COV-2) total Ig and dIgA antibodies in human plasma. Test time is 30 mins and 10 uL plasma is required to perform the assay, although the assay may be modified for the use of whole blood, including finger-prick whole blood or venous whole blood, by methods well known in the art.

The SARS-COV-2 (COVID-19) total Ig and dIgA test is a lateral flow immunochromatographic assay for the detection of COVID-19 total Ig and dIgA antibodies present in human plasma. Spike antigen (“T1” test line 1) and CSC (“T2” test line 2) are immobilized onto a nitrocellulose membrane, and biotinylated Spike antigen labelled with streptavidin gold detects COVID-19 total Ig and dIgA in the patient's plasma, which are visualized as pink/purple lines. A procedural control is included in the test to determine that the assay has been run correctly. The test kit is stored between 20° C. and 25° C. The components should not be frozen.

Plasma sample is required for this assay. The plasma is centrifuged at 10,000 RPM for 5 minutes prior to testing. Samples should be run as soon as possible after collection and kept at or below 8° C. at all times. Samples can be stored at 2-8° C. for 7 days if not run immediately. If long-term storage is required, they should be stored at −20° C. for periods less than 3 months, or store at −80° C. for periods longer than 3 months. Avoid repeated freezing and thawing. Running buffer, test devices and samples are equilibrated to ambient temperature before running the test.

### Assay Procedure

### Step 1

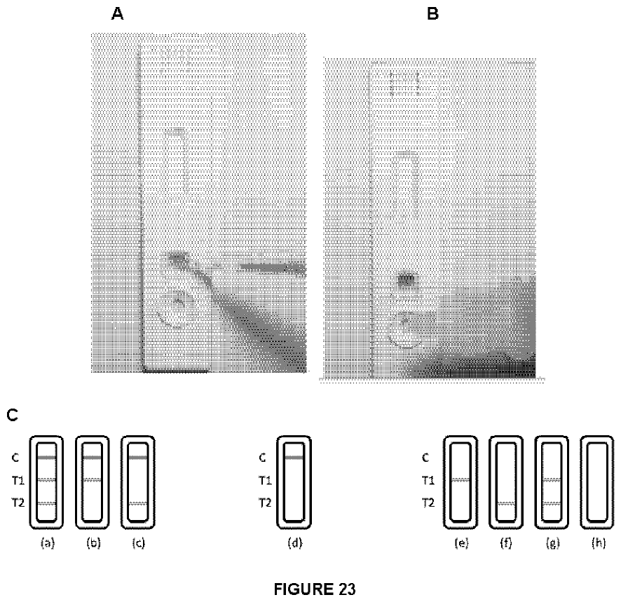

Remove the lateral flow device from the sealed bag prior to use.
Carefully collect 10 uL of plasma with a micropipette.
Allow the pipette tip to touch the center of the well A (top well FIG. 23A) and dispense.
Immediately go to step 2.

### Step 2

Hold the running buffer bottle vertically 1 cm above the well B (bottom well FIG. 23 B), add 4 drops of running buffer to the well B. Allow each drop to absorb into the pad prior to next drop addition.
Set the timer and wait 30 minutes.

### Step 3

Immediately read results after 30 minutes by visual interpretation as shown in FIG. 23 C and Axxin Reader.

### Example 11—Measurement of Plasma dIgA as a Marker of Time Since Infection and as a Surrogate of Mucosal SIgA Responses

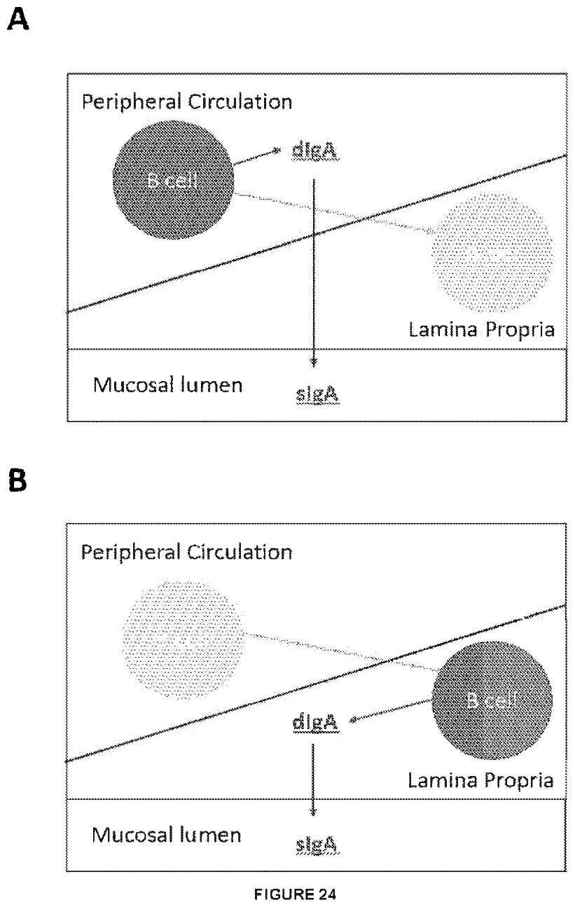

In the context of measuring immune responses that can provide immunity to mucosally transmitted pathogens such as SARS COV-2, mucosal antibody and especially mucosal SIgA are likely to be most relevant, however the measurement of mucosal SIgA is problematic because of inconsistent sample collection for specimens such as saliva, nasal washings, lung washing, fecal washing etc. shows the advantage of sampling whole blood for dIgA as a marker of mucosal immune response in the early convalescent phase or early after immunization, rather than sampling mucosal surfaces such as saliva for SIgA. FIG. 24 demonstrates how measurement of dIgA in plasma can provide a more accurate surrogate of SIgA in mucosal samples. A) Early after infection or immunisation, B-cells producing pathogen-specific dIgA are mobilized and expanded in the peripheral circulation, excreting pathogen-specific dIgA into the blood where it can readily be sampled and detected using the methods described herein. The dIgA produced in the circulation is exported as SIgA when it comes into contact with mucosal epithelia, and will become undetectable in plasma once all the relevant B-cells migrate to the mucosal lamina propria. The amount of pathogen-specific dIgA in the peripheral circulation at this time, including the AUC, will reflect the number and functional capacity of such B-cells that are destined to migrate to the mucosal lamina propria. Exported SIgA can be detected in saliva and other mucosal samples at this time, but is subject to the inconsistent sampling errors and dilution in these samples. B) Later after infection or immunization, the dIgA-producing B cells have homed to the mucosal lamina propria, where they continue to produce pathogen-specific dIgA but because of their close location to the epithelia, the dIgA is efficiently exported as SIgA and does not enter the peripheral circulation in detectable amounts. B-cells can be detected in the tissue, and exported SIgA can be detected in saliva and other mucosal samples at this time, but both of these measures are subject to inconsistent sampling errors, sample dilution and inconvenience of tissue sampling.

### Example 12—Anti-SARS-COV-2 ELISA (dIgA)

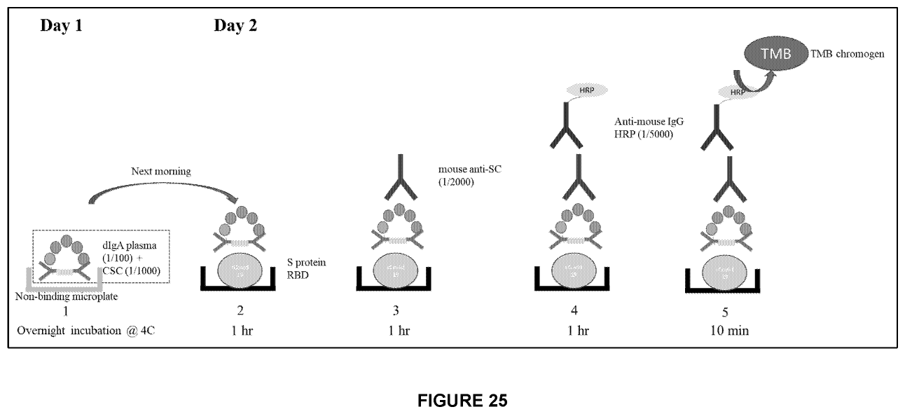

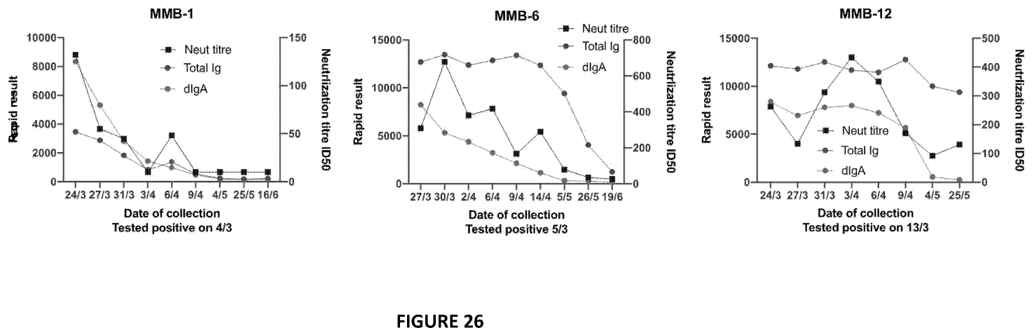

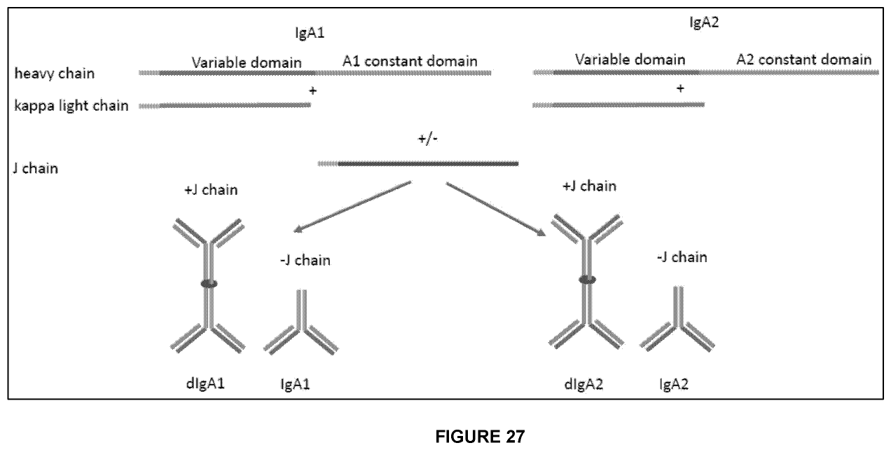

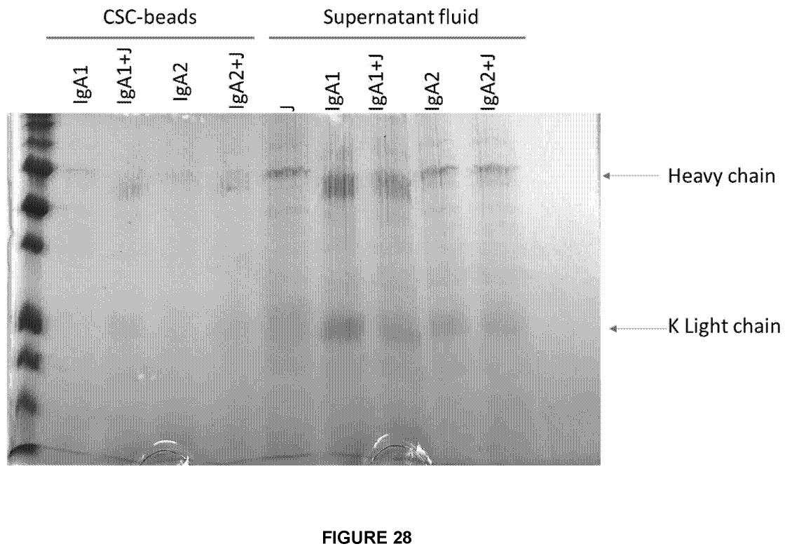

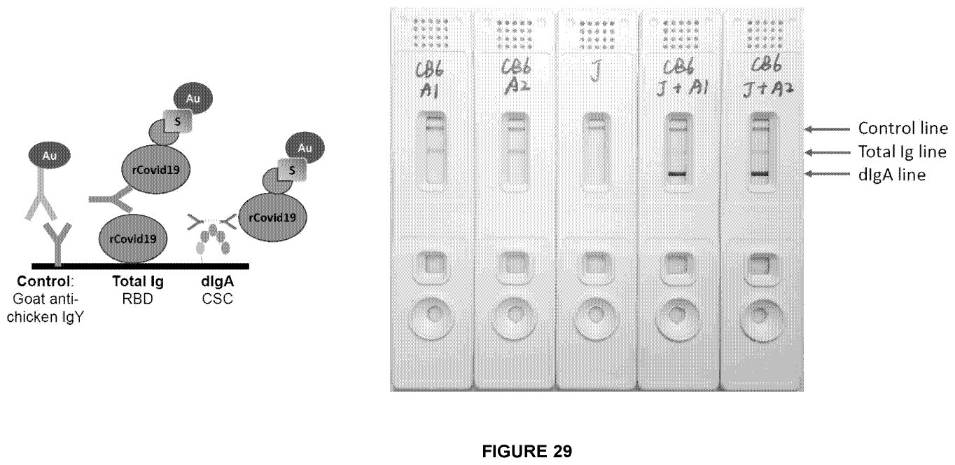

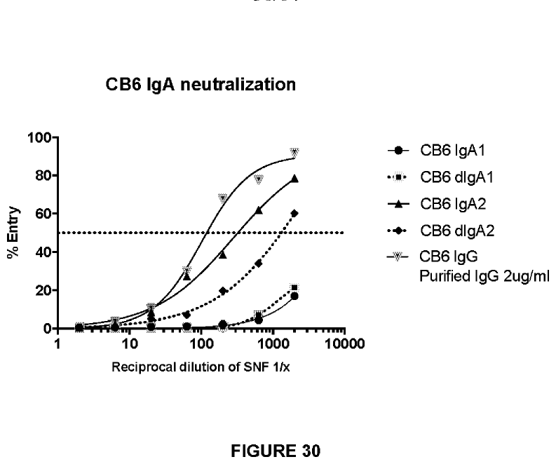

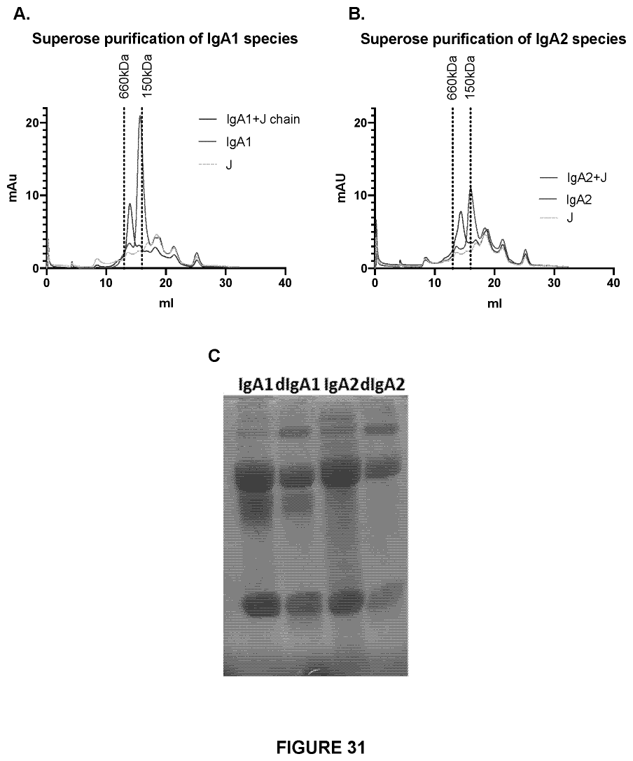

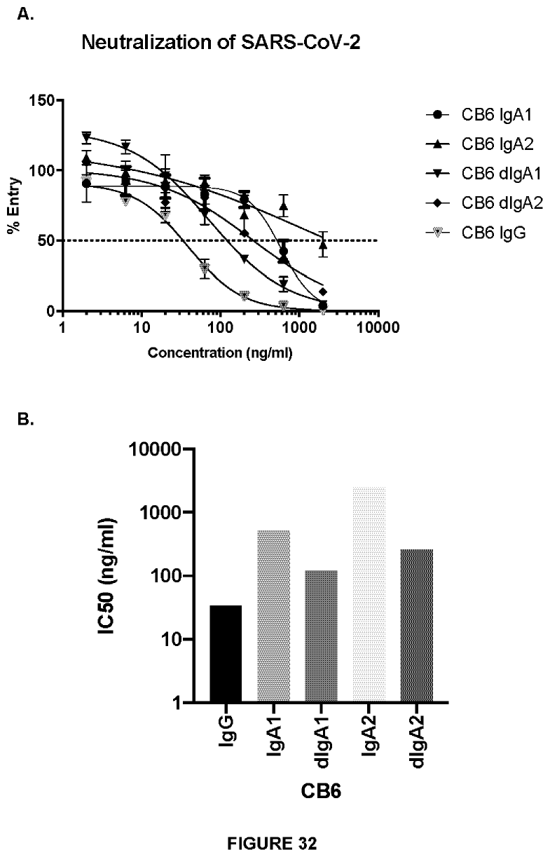

The test kit is coated with recombinant SARS-COV-2 Spike protein (RBD, His Tag) or a Spike antigen as described herein. In the first complexing step, the Chimeric Secretory Components (CSCs) bind to dimeric IgA (dIgA) of the diluted patient samples. Specific dIgA (also IgG and IgM) antibodies in positive samples, will bind to the antigen. To detect the bound dimeric IgA, a 1° detecting antibody will bind to the CSC of the CSC/dIgA complex. A labelled anti-mouse IgG (enzyme conjugate) is then added to detect the bound antibodies, catalysing a colour reaction. The reagent wells were coated with the SARS-COV-2 Spike protein (RBD, His Tag), GenScript catalogue #Z03483. The principle of the assay is diagrammatically shown in FIG. 25.

| Component | | Format | |
|---|---|---|---|
| 1. | Microplate wells coated with antigens: 12 microplate | 12 × 8 | |
| | strips each containing 8 individual break-off | | |
| | wells in a frame ready for se | | |
| 2. | Calibrator: (dIgA, human) | 5 | uL |
| 3. | Positive control: (dIgA, human) | 5 | uL |
| 4. | Negative control: (dIgA, human) | 5 | uL |
| 5. | CSC reagent: Chimeric Secretory Component | 20 | uL |
| 6. | 1° Detecting Antibody: Mouse | 10 | uL |
| | anti-secretory component antibody | | |
| 7. | Enzyme conjugate (ab97023): | 5 | uL |
| | (Goat anti-mouse IgG HRP) | | |

| | | |
|---|---|---|
| 8. | Sample Buffer: 1% BSA in PBS + 0.05% | Not |
| | Tween, ready for use | provided |
| 9. | Wash Buffer: (1 × PBS + 0.05% Tween) | Not |
| | | provided |
| 10. | Chromogen/substrate solution | Not |
| | (SeraCare Cat#: 5120-0083): | provided |
| | TMB/H₂O₂, ready for use | |
| 11. | Stop Solution. 0.5M Sulphuric acid, read for use | Not |
| | | provided |
| 12. | Protective seal | 2 pieces |
| 13. | Low/non-binding microplate | 1 plate |

Unopened kit may be stored for up to 6 months at 2° C. to 8° C. Once opened, microplate wells and other reagent can be stored up to 1 month at 2° C. to 8° C. Unused well strips should be kept in a sealed bag with the desiccant provided to minimise exposure to damp air.
Preparation of the controls, calibrators, and samples
CSC reagent: CSC reagent is diluted 1:1000 in sample buffer. For example: dilute 1 uL CSC reagent in 999 uL sample buffer and mix well by vortexing.
Calibrator and controls: Positive, negative and calibrator controls are diluted 1:100 in CSC reagent. For example: dilute 10 uL controls or calibrator in 990 uL CSC reagent and mix well.
Sample preparation: Patient samples are diluted 1:100 in CSC reagent. For example: dilute 10 uL serum in 990 uL CSC reagent and mix well.

### Preparation of the Reagents

Note: All reagents must be brought to room temperature (+18° C. to 25° C.)
Coated wells: Ready for use.
1 Detecting Antibody: Detecting Ab is diluted 1:2000 in sample buffer.
Enzyme conjugate: anti-mouse IgG HRP is diluted 1:5000 in sample buffer.
Sample buffer: Ready for use.
Wash buffer: Ready for use
Chromogen/substrate solution: Ready for use. Keep in the dark, as the contents are sensitive to light.
Stop solution: Ready for use.

### Assay Procedure

### Day 1:

1. Sample complexing: Prepare 150 uL of the calibrator, positive and negative controls or diluted patient samples with CSC reagent into low/non-binding microplate wells according to the pipetting protocol. cover the plate with a protective seal and incubate overnight at 4° C.

### Day 2:

2. Sample incubation: Remove the protective seal. Mix by pipetting a few times and transfer 100 μL of the complexing samples into the individual microplate wells coated with the COVID-19 antigen according to the pipetting protocol. cover the plate with a protective seal and incubate for 60 minutes at 37° C.
3. Washing: Aspirate the liquid from each well and wash 6 times. Wash by adding approximately 300 uL of Wash Buffer using a squirt bottle, multi-channel pipette, manifold dispenser or automated washer. After the last wash, remove any remaining wash buffer by inverting the plate and tap against clean absorbent paper.
4. 1° Detecting Ab incubation: Pipette 100 uL of 1° detecting Ab into each well. Cover the plate with a protective seal and incubate for 60 minutes at 37° C.
5. Washing: Aspirate and wash the wells as outlined in Step 3.
6. Conjugate incubation: Pipette 100 uL of enzyme conjugate into each well. Cover the plate with a protective seal and incubate for 60 minutes at 37° C.
7. Washing: Aspirate and wash the wells as outlined in Step 3.
8. Substrate: Pipette 100 uL of chromogen/substrate solution into each well and incubate for 10 minutes at room temperature protected from direct sunlight.
9. Stopping: Pipette 100 uL of stop solution into each well in the same order and speed as the chromogen/substrate solution.
10. Measurement: Read plate using wavelength 450 nm and reference wavelength between 620 nm and 650 nm.

### Calculation of Calibrator/Cut-Off

Average the duplicate readings of the calibrator optical density and multiple 0.8.

### Cut-Off

(OD1(cut-off)+OD2(cut-off))×0.8=Cut-off control value

For Example: (0.42+0.40)/2)×0.8=0.33 cut-off control value

A positive result (COVID19 dIgA) is when the test value is equal to or above the cut-off control value. A negative result is when the test value is less than the cut-off control value.

### Example 13—Development of a Rapid and Sensitive Lateral Flow Assays to Measure dIgA to SARS-COV-2 in Whole Blood or Plasma or Serum Samples

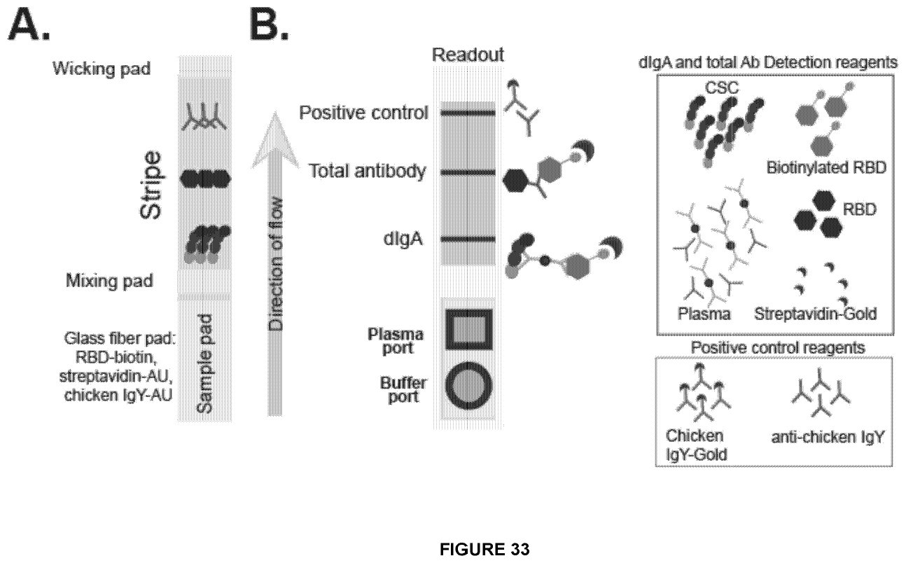

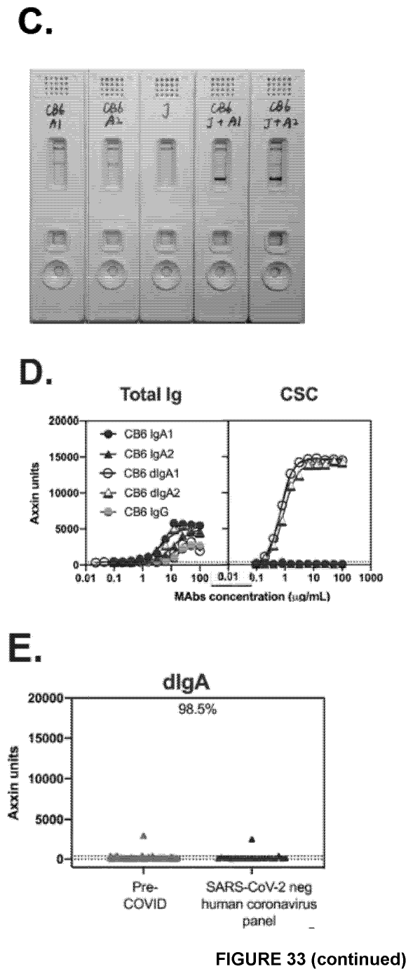

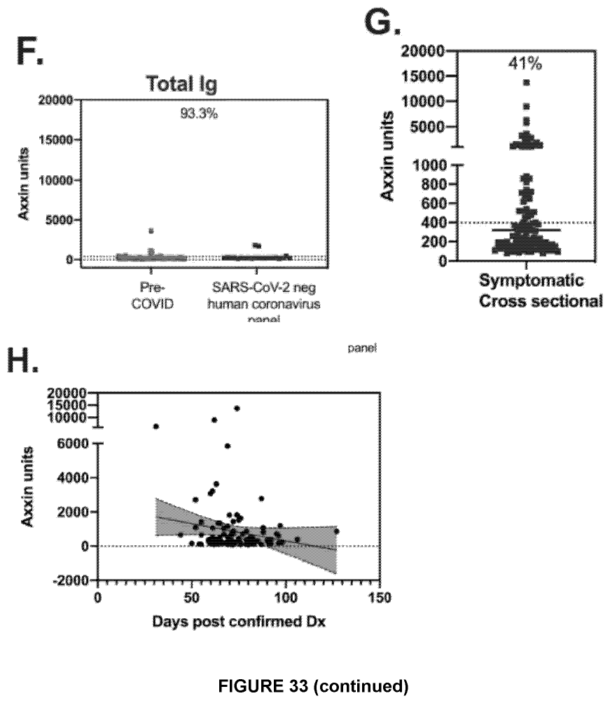

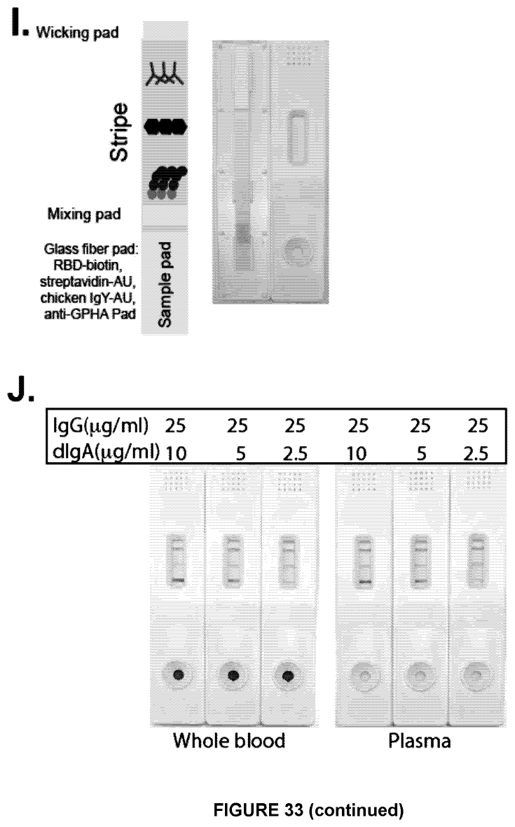

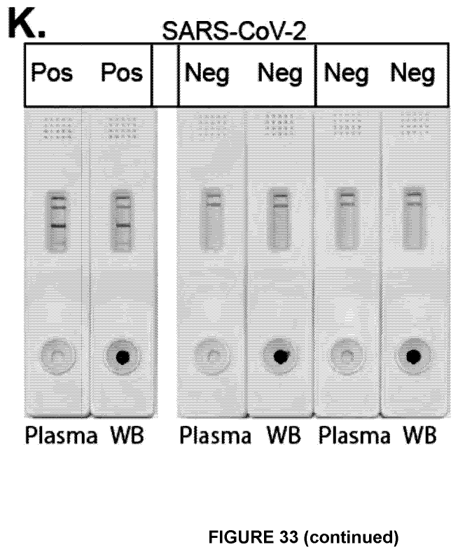

A lateral flow test was developed to rapidly detect the presence of dIgA specific to the receptor binding domain (RBD) of SARS-COV-2 in plasma or whole blood. The test is based on the inventors' earlier work showing the ability of a recombinant chimeric secretory protein (CSC) to specifically bind only dIgA, not monomeric IgA, IgG or IgM. Nitrocellulose was striped with (a) CSC for the detection of dIgA, (b) RBD for the detection of total anti-RBD antibodies, and (c) anti-chicken IgY as a procedural control (FIG. 33A). Glass fiber pads were impregnated with RBD-biotin, streptavidin-Au (40 nM) and chicken IgY-Au (40 nM) and dried. Plasma samples (15 ul) were applied to the sample port of LFA devices followed by buffer into the buffer port. dIgA antibodies specifically binding RBD-biotin/streptavidin gold complexes are captured by the CSC stripe, while total RBD specific immunoglobulins specific to RBD are captured on the RBD stripe (FIG. 33A). Chicken IgY-Au is captured on the anti-chicken IgY line and confirms the flow of buffer through the entire nitrocellulose strip (FIG. 33B). The assay allows for visual interpretation of the presence of RBD specific dIgA and can optionally be read using a lateral flow reader (Axxin AX-2XS, Axxin, Melbourne). A visible line here corresponds to ≥400 Axxin reader units.

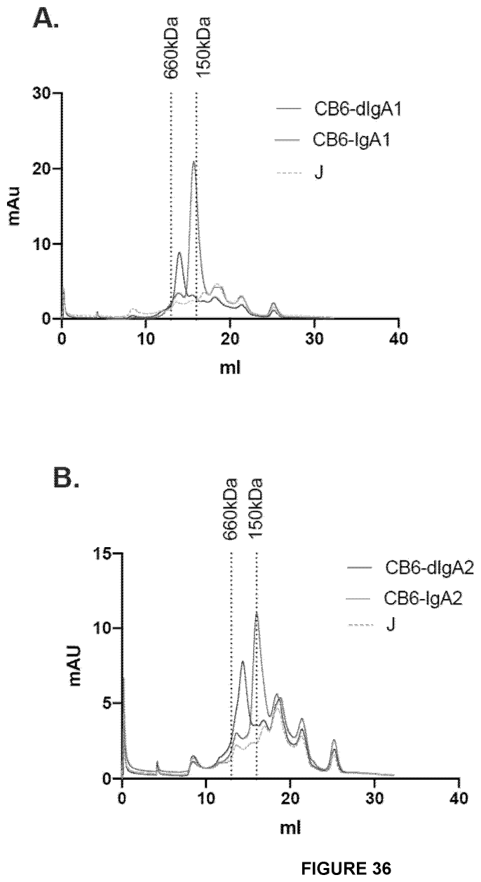

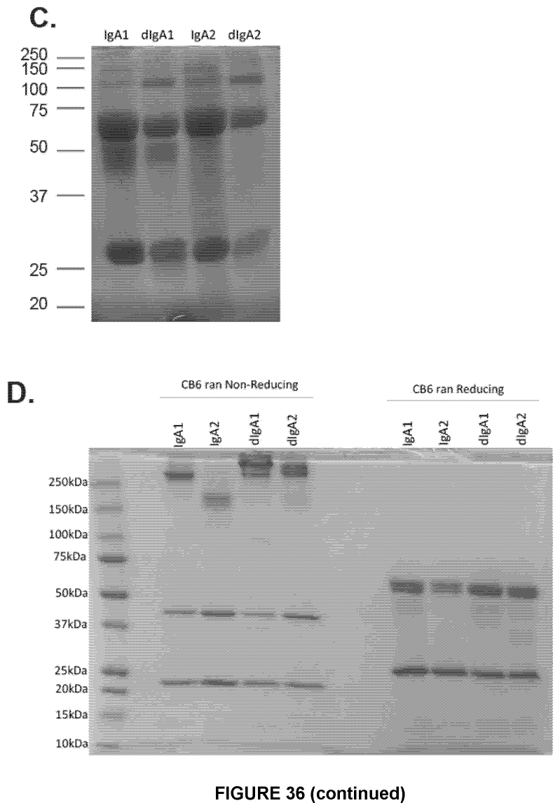

The assay format was validated and only detects RBD-specific dIgA and not monomeric IgA. This was undertaken by converting the Fc portion of the SARS-COV-2 specific monoclonal antibody CB6 (Shi, R. et al. A human neutralizing antibody targets the receptor-binding site of SARS-COV-2. Nature 584, 120-124 (2020)) to the IgA1 and IgA2 Fc sequence, and co-expressing the CB6 kappa chain, IgA1 or IgA2 heavy chain with J chain for dimeric IgA production, or without the J chain for IgA production and purified using either ammonium sulfate precipitation and gel filtration, or via protein L affinity chromatography (FIGS. 35 and 36). Dimeric IgA1 and dIgA2 species eluted as 300 kDa dimers in size exclusion chromatography and migrated as higher molecular weight species compared to their monomeric IgA1 and IgA2 counterparts, indicative of covalent dimerization with the J chain (FIG. 36).

The tissue culture fluid of transfected cells expressing CB6-IgA1, CB6-dIgA1, CB6-IgA2, CB6-dIgA2 and J chain alone were applied to the LFA devices and showed specific detection of only dIgA1 and dIgA2 at the dIgA (CSC) test line (FIG. 33C). Next CB6-IgG, CB6-IgA1, CB6-dIgA1, CB6-IgA2, CB6-dIgA2 were purified and applied as serial dilutions of the proteins to the devices and the intensity of the lines was measured in a lateral flow reader. The results showed highly specific detection of both dIgA1 and dIgA2 with a limit of detection of 0.123 and 0.128 μg/ml respectively, but not IgA1, IgA2 or IgG even at the highest concentrations of purified antibody (100 μg/ml) (FIG. 33D). At concentrations of dIgA that were above 3-6 g/ml, saturation of the signal was observed; there was, however, a clear dose response within the range of 0.04-3 μg/ml. Each of IgA1, IgA2, dIgA1, dIgA2 and IgG antibodies were detectable in the total antibody (RBD) test, but with less sensitivity than for dIgA via CSC capture (limit of detection of 1.0, 1.6, 4.4, 0.7 and 5.0 μg/ml, respectively).

To examine assay specificity, 171 pre-COVID-19 plasma samples, collected prior to December 2019, were examined. Of the 171 plasma samples tested, 3 were positive for SARS-COV-2 RBD-specific dIgA by LFA providing specificity of 98.2% (FIG. 33E). Two samples were excluded from the dIgA analysis due to the presence of anti-streptavidin antibodies (not shown). For total Ig in the lateral flow test, of the 171 pre-COVID-19 samples, 10 recorded a positive result providing specificity of 94.2% (FIG. 33F).

In addition, 22 plasma samples from PCR confirmed SARS-COV-2 negative patients with either current or previous non-SARS-COV-2 coronavirus infection were examined to determine potential cross-reactivity with seasonal coronavirus infections. Of these 22 plasma samples, IgG reactivity towards 229E, HKU1, NL63, and OC43 was detected in 16, 21, 20, and 18 samples, respectively (Table 4). Evidence of recent or active infection with either 229E, NL63, or OC43 was detected in 1, 1, and 2 samples respectively, as determined by PCR. Using the lateral flow device, one sample returned a dIgA positive result (specificity 95.5%), while 3 total Ig positives were recorded from the non SARS-COV-2 coronavirus panel (specificity 86.4%). None of these false positives had current PCR confirmed coronavirus infection; 2 false positive results were from individuals with HIV infection of which one had PCR confirmed parainfluenza infection.

Next, 112 cross-sectional samples obtained from community sourced cases of PCR-confirmed SARS-COV-2 infection were assayed for the presence of dIgA specific to RBD. Plasma samples were collected between 31-127 days post-PCR confirmation of infection and were screened for the presence of RBD-specific dIgA using the LFA. Forty plasma samples were positive for the presence of dIgA (41%) and correlated negatively with time post diagnosis, suggesting that dIgA declined rapidly during convalescence or after viral clearance (FIGS. 33G and H).

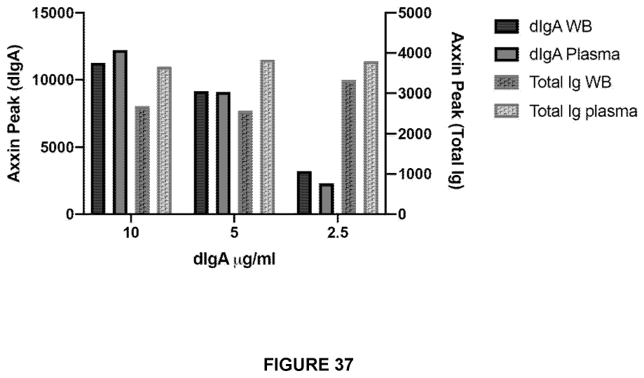

One intended use of the assay is to detect evidence of seroconversion to contagious agents such as SARS-COV-2 in whole blood. In one embodiment, the assay was adapted to allow the separation of red blood cells and plasma through inclusion of an anti-glycophorin A (GPHA) pad over the sample pad. Red blood cells are agglutinated via anti-GPHA and after addition of buffer, plasma flows via capillary action along the nitrocellulose membrane (FIG. 331). The detection of dIgA and total Ig was compared using whole blood spiked with a constant amount of CB6 IgG plus varying amounts of dIgA and similar levels of detection detected in both configurations (FIG. 33J). Plasma from a SARS-COV-2 early convalescent subject was spiked with red blood cells to mimic whole blood and compared to plasma alone; similar levels of both dIgA and total Ig were detected. Two plasma samples collected prior to December 2019 were similarly reconstituted as whole blood or used as plasma alone and recorded negative results to both dIgA and total Ig (FIG. 33K). Quantitation was performed on an Axxin reader and is shown in FIG. 37. This demonstrates proof-of-concept that the SARS-COV-2 dIgA test can be used on whole blood to detect dIgA and total Ig to SARS-CoV-2 RBD.

### Example 14—Dimeric IgA is an Early Marker of Seroconversion to SARS-COV-2

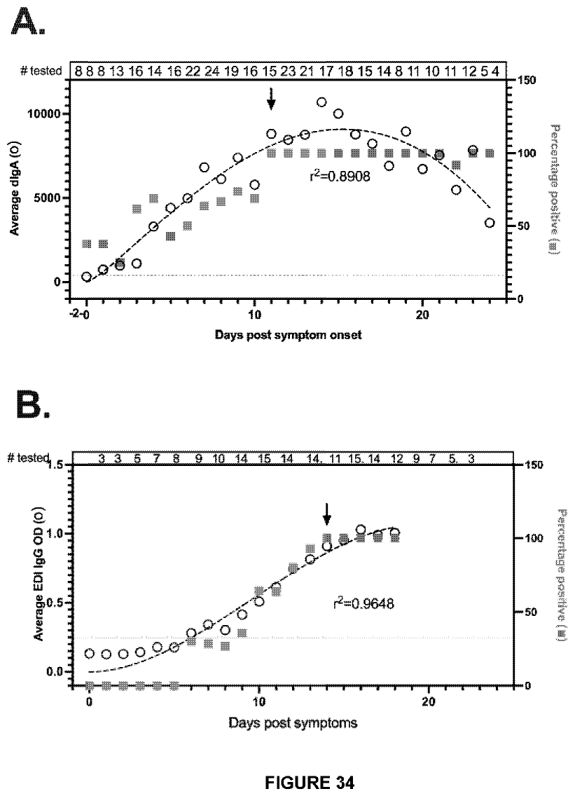

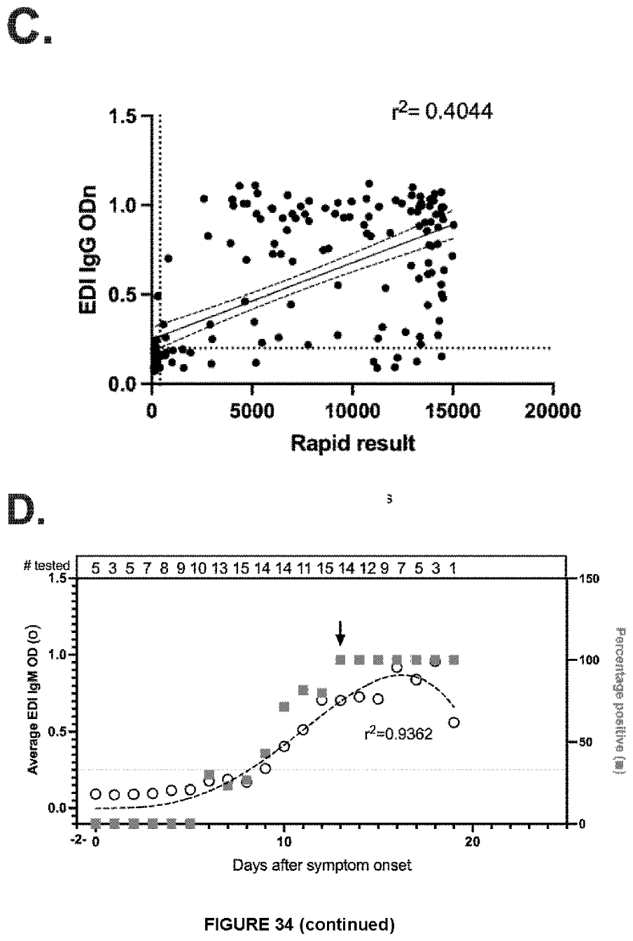

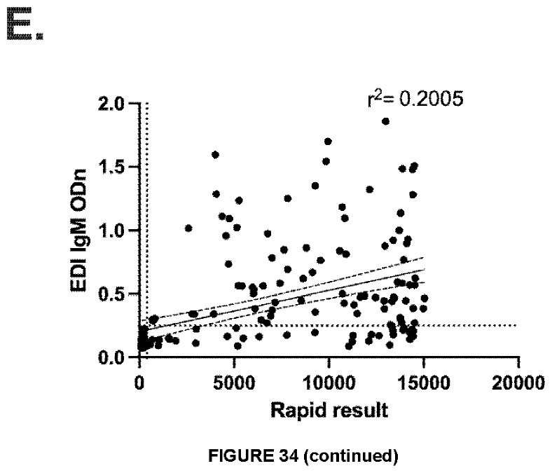

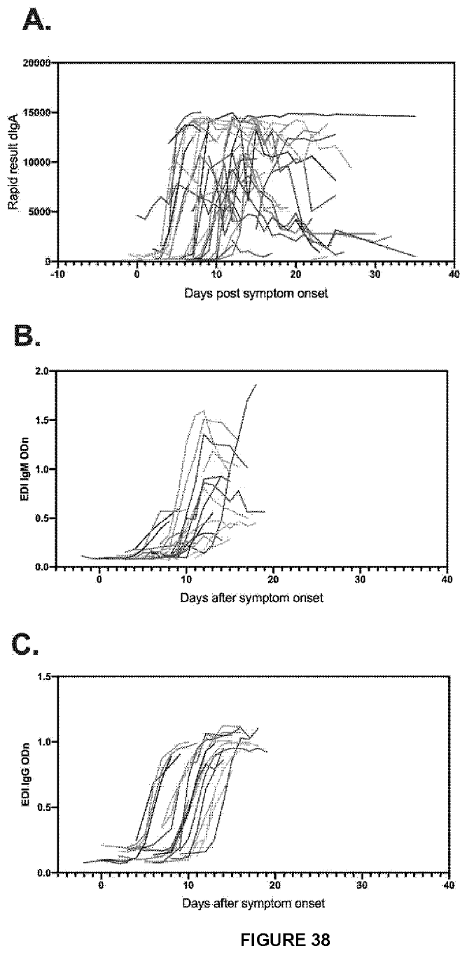

To investigate the timing of detectable dIgA following exposure to SARS-COV-2, we examined dIgA levels over time in plasma samples from 45 subjects with PCR confirmed SARS-COV-2 infection collected from −2 to 36 days after symptom onset with the majority of samples collected either 8 days before or after symptom onset (FIG. 38). Samples were analysed for the presence of dIgA using the rapid LFA test (described above) and intensity was measured in a lateral flow reader (FIG. 34A). In total, dIgA data were obtained for 362 plasma samples, with 25 samples omitted due to insufficient sample volume.

Results indicate that between 0 and 10 days after COVID-19 symptom onset, there was a steady increase in the levels of dIgA as well as the number of subjects who tested positive (>400 Axxin units) for dIgA (FIG. 34A and FIG. 38), with 100% of subjects having detectable dIgA by day 11 post-symptom onset, with an average of 8817 units. Peak levels of dIgA were detected between day 14 and 15 after symptom onset. Overall, dIgA was detected in 49% of people between 0-5 days, 65% between days 6-10, and 100% from day 11 onwards post-symptom onset (Table 3). While the number of subjects testing positive was maintained over 25 days, the average level of dIgA steadily declined from day 15 onwards.

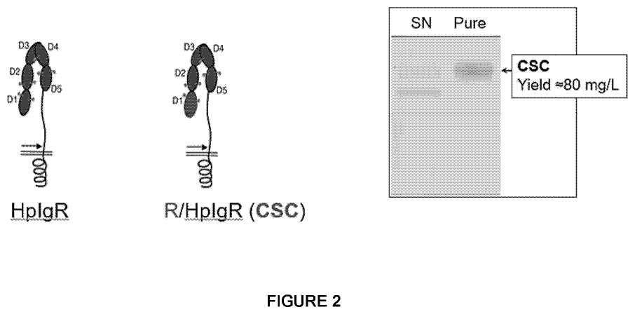

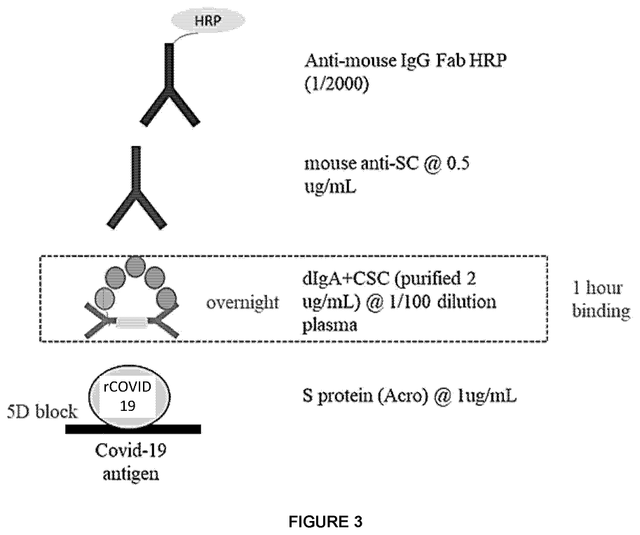

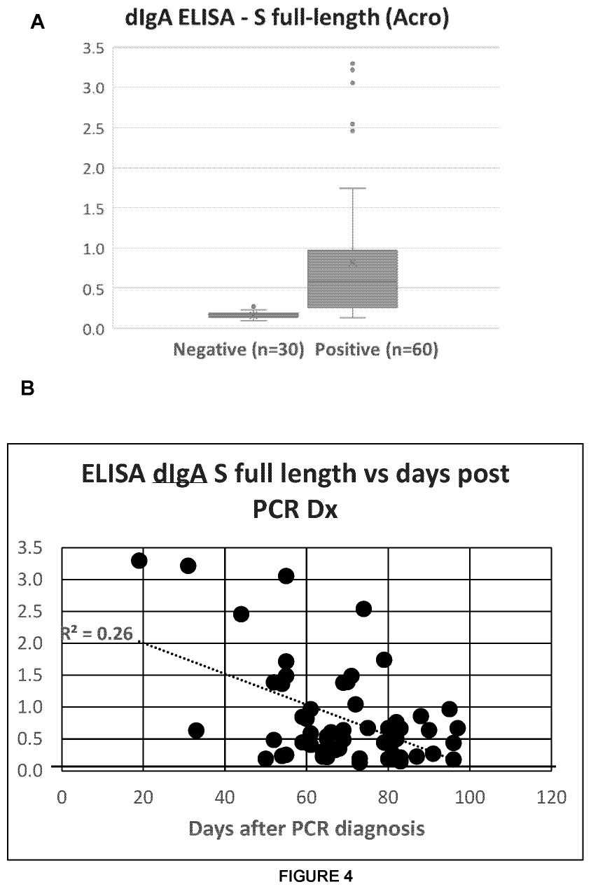

The same subjects were analysed for the presence of IgG and IgM specific to nucleocapsid (N) antigen. The test utilised was reported to be able to detect IgM in 5.9% of people ≤5 days after symptom onset, 37.1% for IgM between 5-10 days, 76.4% for IgM 10-15 days, and 94.4% for IgM after days 15-22. The reported “false” positivity rates were 0.5% for IgM in healthy blood donors and 1.6% for IgM in ICU patients (Bundschuh, C. et al. Evaluation of the EDI enzyme linked immunosorbent assays for the detection of SARS-COV-2 IgM and IgG antibodies in human plasma. Clin Chim Acta 509, 79-82 (2020). Likewise, for IgG, 37.1%, 82.4% and 100% of samples were reported to test positive between days 5-10, 10-15 and 15-22, respectively, with a false positive rate of 1% and 1.2% for healthy blood donors and ICU patients, respectively (Bundschuh, 2020 above). In this study, none of the samples tested were positive for SARS-COV-2 anti-N IgG or IgM in the first 5 days (FIGS. 34B and 2D and FIG. 38). Between days 6-13, there was a steady increase in the number of samples testing positive, and by day 13 (IgM) and day 14 (IgG), all samples tested were positive for antibody against SARS-COV-2 N protein. Overall, between days 0-5, 6-10 and 11-15, 0%, 39% and 92% of samples tested positive for IgM and 0%, 34% and 67% tested positive for IgG, respectively (Table 3). While dIgA and IgG were moderately correlated r²=0.4044 (p<0.0001) (FIG. 34C), dIgA and IgM were relatively weakly correlated, r²=0.2005 (p<0.0001) (FIG. 34E).

Taken together, these data show that dIgA is an important serological marker of SARS-COV-2 infection, and that dIgA is generated earlier than both IgM and IgG, and at a time coinciding with reduced sensitivity of molecular and antigen testing as viral levels lower.

### Example 15—Half-Life Analysis of dIgA

The ability to detect dIgA earlier than either IgG or IgM after SARS-COV-2 infection suggests it may have complementary utility to antigen or PCR-based detection of active SARS-COV-2 especially for example where suspected cases return a negative result in those assays, in order to increase screening sensitivity and for backward contact tracing to identify recently infected antigen- or PCR-negative individuals. Therefore, the half-life of dIgA was determined to understand how long dIgA persists in plasma and to provide a window of infection recency using a well characterised seroconversion panel with at least 4 samples available at different times after infection. Five of the 6 subjects tested were dIgA positive and were used to calculate the half-life. A one-phase decay equation was used to calculate the half-life of dIgA observed in the rapid LFA with an average R2 of 0.9862. The mean interval between first and last sample included for calculation was 62 days. The half-life of dIgA was 6.3 days with lower and upper 95% confidence intervals of 3.6 and 9.6 days, respectively.

A similar analysis performed for the total immunoglobulin line of the LFA test revealed that 5 of these 6 individuals had a detectable total immunoglobulin positive result, of which 1 became negative within the observation period at day 19 post PCR. The levels of total Ig declined at a slower rate over the sampling period compared to dIgA and a broad range of half-life values was calculated, with low confidence (average r²=0.88) ranging from 6.4 to 207,143 days. By contrast, the anti-S1 IgG levels in these same samples remained relatively steady and did not fit the one phase decay nor a continuous decay equation.

### Example 16—Discussion of Results

Using a longitudinal panel of 362 samples sourced from −2 to 36 days after symptom onset from 45 subjects, it was determined that between days 0-5, dIgA was detectable in 49% of samples, reaching a peak of 100% detection by day 11. By contrast, IgM, usually considered an early marker of seroconversion, was absent before day 5, and only detected in 39% of subjects between days 6-10, reaching a maximum of 92% detection between days 11-15. This was not dissimilar to IgG with 34% detection between days 6-10 and 67% between days 11-15. Comparison with IgA responses in the same subjects revealed that whilst the trajectory was similar, IgA appeared to be more stable in the plasma and reached assay saturation in many subjects. By contrast, the dIgA LFA responses appear spread over a wide dynamic range and were consistently recorded after 11 days post symptom onset with a measurable decline in the level of reactivity beyond 20 days. One subject in particular did not generate a detectable IgA response at either time point tested but was strongly positive for dIgA.

Dimeric IgA is actively transported across mucosal surfaces by binding to pIgR on the basolateral surface of epithelial cells. After transcytosis, pIgR is cleaved at the apical surface to release SIgA covalently bound to SC. This process actively depletes dIgA from plasma over time, concentrating dIgA instead to the mucosa where it protects against infection by pathogens entering via mucosal surfaces. Analysis of the large longitudinal cohort determined that the levels of dIgA appear to begin to decline after approximately day 20. This cohort extended to at most day 35 post symptom onset, with many patients exhibiting a plateau of dIgA reactivity through the period of sampling (FIG. 38). To examine the half-life of dIgA, two extended longitudinal panels were analysed where there were at least four data points showing a decline in dIgA levels. Six subjects were included in this analysis and revealed that the half-life of RBD-specific dIgA in serum was 6.3 days. A recent study using 844 samples collected from longitudinal samples from 151 RT-PCR positive convalescent SARS-COV-2 patients showed that the half-life of RBD specific IgG antibodies was 62 days (Steenhuis, M. et al. Dynamics of antibodies to SARS-COV-2 in convalescent plasma donors. medRxiv (2021), while a separate study of 51 patients reported even longer half-lives of spike-specific IgG in paired serum samples of 103 days and for IgA 210 days (Dan, J. M. et al. Immunological memory to SARS-COV-2 assessed for up to 8 months after infection. Science 371 (2021)). While the observed short half-life of SARS-CoV-2 specific dIgA precludes its use for measuring past infections beyond a few months, it provides highly sensitive detection in the early phase after infection (peaking at 100% between 11-35 days) and this provides a unique serological biomarker for recent infection.

Serology for many viral infections relies on the detection of IgM as a marker of acute or current infection, with IgG as a marker of past infection, and the early part of the COVID-19 pandemic saw a profusion of commercially available laboratory and POC tests designed for the measurement of SARS-COV-2 specific IgG and IgM. However, IgM has many limitations in serology especially with low-prevalence infections (See for example, Landry, M. L. Immunoglobulin M for Acute Infection: True or False? Clin Vaccine Immunol 23, 540-545 (2016)), and none of these COVID-19 IgG/IgM tests have proven to be useful for determining the timing of recent or past infections in the absence of direct virus detection, severely limiting their utility in public health control measures.

The results presented in this application demonstrate that SARS-COV-2 specific dIgA provides this missing tool for COVID-19 serology. The results presented in this application demonstrate that contagious agent specific dIgA is likely to provide a missing tool for reliable, sensitive serology.

Rapid antigen detection tests have received emergency authorization use under the FDA and have shown high specificity (>99%). However, the sensitivity of such tests is highly variable in symptomatic patients (63.7-79%) and have highest sensitivity (94.5%) where there is a high viral load (Ct values≤25 or >106 genomic virus copies/mL) early in infection (<7 days) (Dinnes, J. et al. Rapid, point-of-care antigen and molecular-based tests for diagnosis of SARS-COV-2 infection. Cochrane Database Syst Rev 3, CD013705 (2021). The pIgR based dIgA test performs at its best in the 21^{nd} and 3^{rd} week post symptom onset and a positive result by itself would indicate recent infection. The use of a rapid antigen test and the dIgA POC test in combination could allow rapid mass screening of individuals to identify active and recent past infections within at least the past 21 days, which would greatly enhance contact screening. For example, the dIgA LFA test could be used in backward contact tracing, including well-recognised super-spreader events. In these situations, where one or more individuals later become symptomatic and return a positive nucleic acid or antigen test result, there is often a missing link to the original source of infection because antigen and nucleic acid tests are likely to have fallen below the limit of detection by the time contact tracing is initiated. Wide-spread dIgA testing could be deployed to determine those who have evidence of recent SARS-COV-2 infection to track chains of transmission, identify potential common sources of infection and their onward contacts, and reducing the number of people who need to isolate in order to reduce forward transmission of the virus.

The total immunoglobulin test line showed a relatively average specificity of 93.3%. The reasons for this probably relates to the use of the isolated receptor binding domain in a double sandwich allowing low affinity interactions to be detected, particularly relevant for IgM antibodies. Different arrangements of LFA elements may be tested to improve the specificity of the total Ig line.

The role of IgA in neutralization of SARS-COV-2 virus and its appearance after infection have been documented. Studies report the appearance of IgA as early as 2 days after symptom onset, earlier than IgG or IgM, detected for both nucleocapsid and the RBD. This is consistent with our observations here that dIgA can be detected in 49% of subjects testing PCR positive up to 5 days after symptom onset, and 100% at day 11(15). Serum neutralization titres also correlate more strongly with IgA specific to RBD than anti-RBD IgG titres and purified monomeric IgA is also more potent than IgG at mediating neutralization of SARS-COV-2 (Sterlin, D. et al. IgA dominates the neutralizing antibody response to SARS-COV-2. Sci Transl Med 13 (2021)). Examination of saliva and bronchoalveolar lavage reveal that IgA dominates the neutralization activity, with dimeric IgA detected in bronchoalveolar lavage fluid. Here, Isolation of B cell clones expressing IgA towards the SARS-COV-2 spike protein and the expression in monomeric or dimeric form reveals that dimeric IgA is 3.8-113-fold times more potent than the corresponding IgA monomers at neutralizing pseudoviruses and 15-fold higher for authentic SARS-COV-2 virions (Wang, Z. et al. Enhanced SARS-COV-2 neutralization by dimeric IgA. Sci Transl Med 13 (2021). This suggest that dIgA plays an essential role in mucosal protection against CoV and other viruses that transmit via mucosal surfaces and is a potent contributor to antibody neutralization activity.

The results presented here enable the use of dIgA screening as described herein as a novel and readily detected biomarker of recent SARS-COV-2 infections and with routine modification of other mucosal viral pathogens, with particular utility for contact tracing and potential for improved detection of incident (an occurrence of an infection in the person recently whether or not the person is actually infected (i.e., when a molecular or antigen test for pathogen presence is negative as well as positive) when tested) infections when used in conjunction with rapid antigen tests. IgM has previously been used as an acute infection marker for a range of viral pathogens but has more recently and herein been shown to lack the reliability of response necessary to provide accurate screening tools. This is the case, even when a pIgR based reagent is used to detect IgM. In contrast, dIgA is shown herein to display a much higher signal to cut-off ratio than IgM, in addition to providing a far more reliable indication of a recent infection either alone or together with tests for current infection such as antigen or molecular testing. In one embodiment, dIgA screening as described and enabled herein is used to assess and determine or quantify mucosal immune response in subject to either to infection by a mucosal pathogen or a prophylactic or therapeutic vaccine against the mucosal pathogen (as shown herein and below). In another embodiment, SIgA screening may be used to directly assess and determine or quantify mucosal immune response in subject either to infection by a mucosal pathogen or in response to a prophylactic or therapeutic vaccine against a mucosal viral pathogen (as shown herein and below).

### Example 17

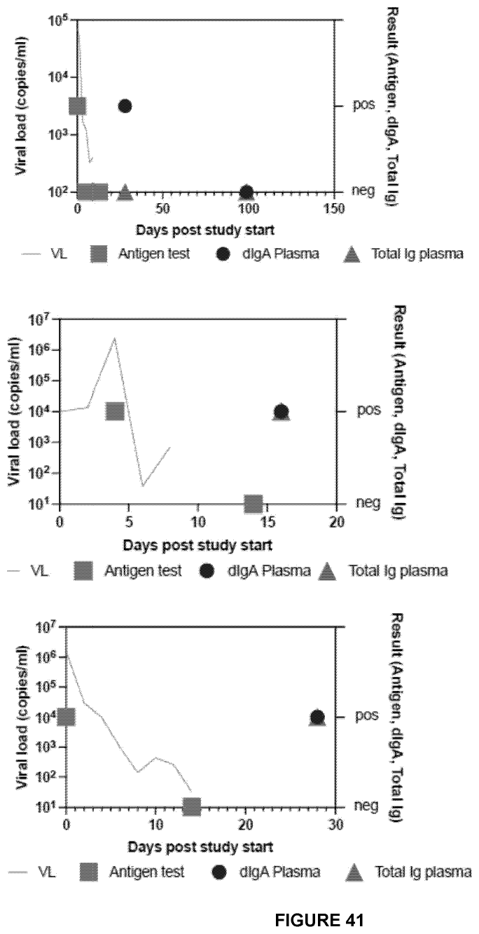

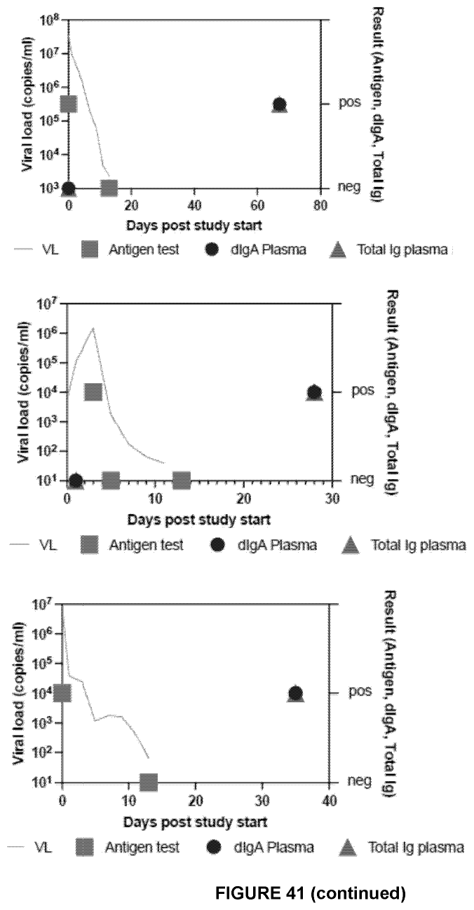

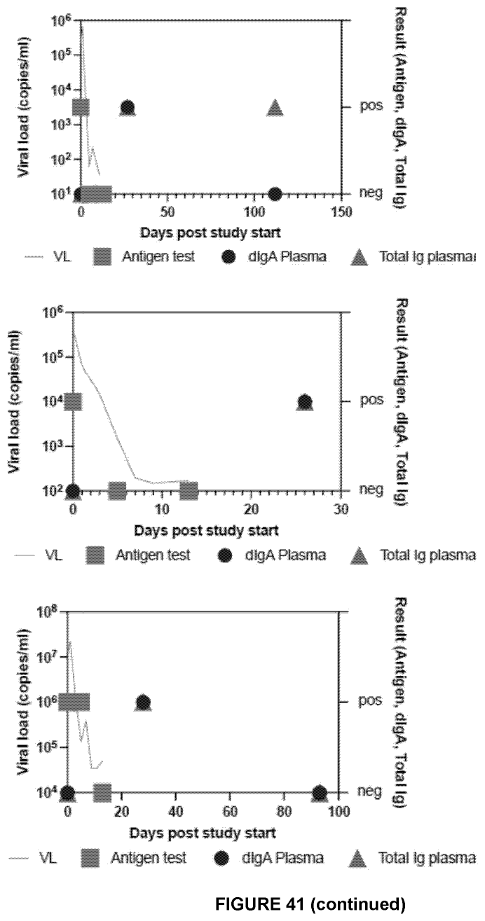

Combined antigen and dIgA testing results provide overlapping and greater screening coverage for recent (including current infections and extending time wise to recent post-current infection) infection than either test alone. Respiratory viral antigen tests rely on the production of large amounts of surface protein such as the nucleoprotein, which generally correlates with the high viral load within the first week of infection. In relation to SARS-COV-2, rapid antigen detection tests have received emergency authorization use under the FDA and have shown high specificity (>99%). However, the sensitivity of such tests is highly variable in symptomatic patients (63.7-79%) and have highest sensitivity (94.5%) where there is a high viral load (Ct values≤25 or >106 genomic virus copies/mL) early in infection (Dinnes J, et al. Rapid, point-of-care antigen and molecular-based tests for diagnosis of SARS-COV-2 infection. Cochrane Database Syst Rev. 2021; 3:CD013705). As determined herein, the dIgA test performs at its best from the 2nd week post symptom onset and a positive result by itself would indicate recent infection. However, the use of a rapid antigen test and the dIgA point of care test in combination would allow rapid mass screening of individuals to identify active and recent past infections reliably within at least the past 28 days, which would greatly enhance contact screening. In this example, see FIG. 41, COVID-19 patients with detectable viral loads by polymerase chain reaction also tested positive for COVID-19 antigen on Day 0 or 3, then tested negative at day 14 when viral loads subsided. An antibody sample collected at day 28 shows the presence of dimeric IgA allowing backward contact tracing of SARS-COV-2 infections in cases where antigen tests and nucleic acid amplification will be negative. The first and last of these panels shows examples of how dIgA disappears by 100 days post infection. Therefore detection of dIgA in plasma is likely to be a result of an infection within the past 100 days.

### Example 18

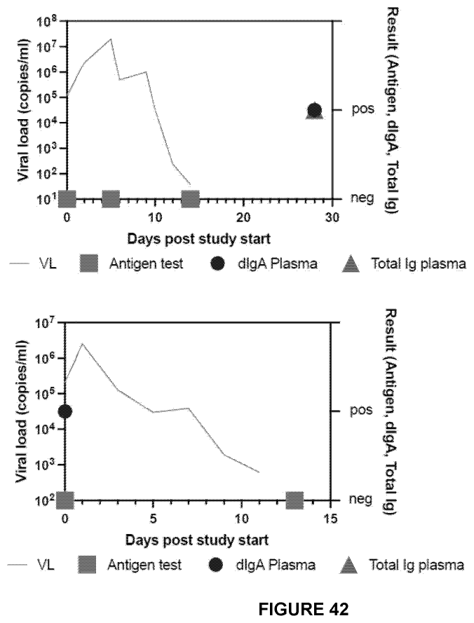

dIgA testing extends the detectability of a positive case in a combined dIgA and antigen test. As shown in FIG. 42, in the left hand side example, antigen tests returned negative results at all three time points tested, despite the presence of nucleic acid. However, dIgA was detectable at day 28 allowing backward contact tracing to occur. In the right-hand example, antigen tests were negative and dIgA was detectable at day 0. The combined use of dIgA and antigen tests in this case would provide a positive diagnosis of recent SARS-CoV-2 infection extending the diagnostic utility of antigen tests when antigen and dIgA rapid tests are used in combination.

### Example 19

Post-vaccination (mRNA or adenoviral vaccines) dIgA responses substantially diminished compared to 100% positive dIgA at day 11 after infection.

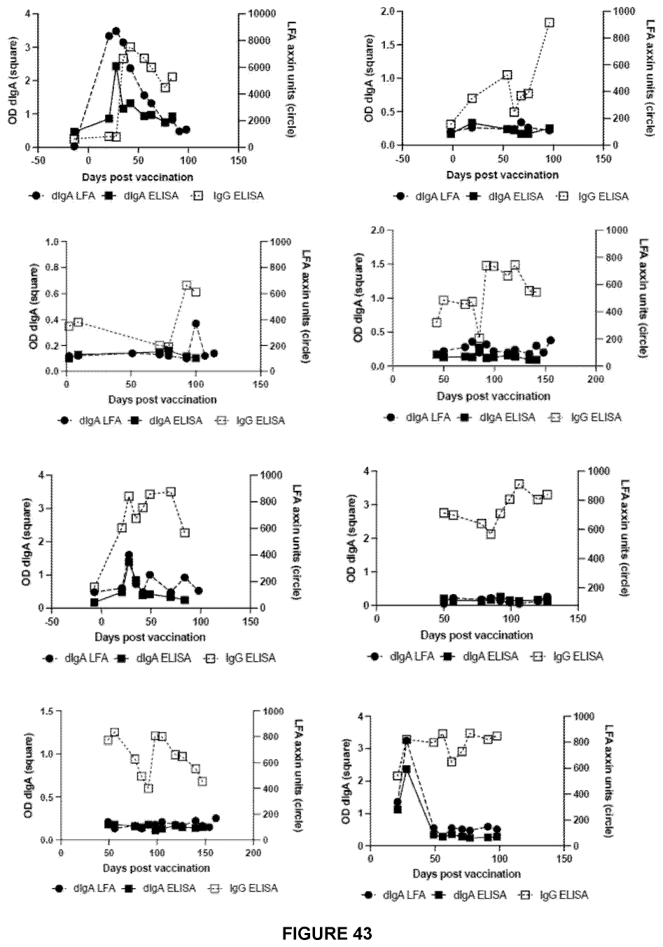

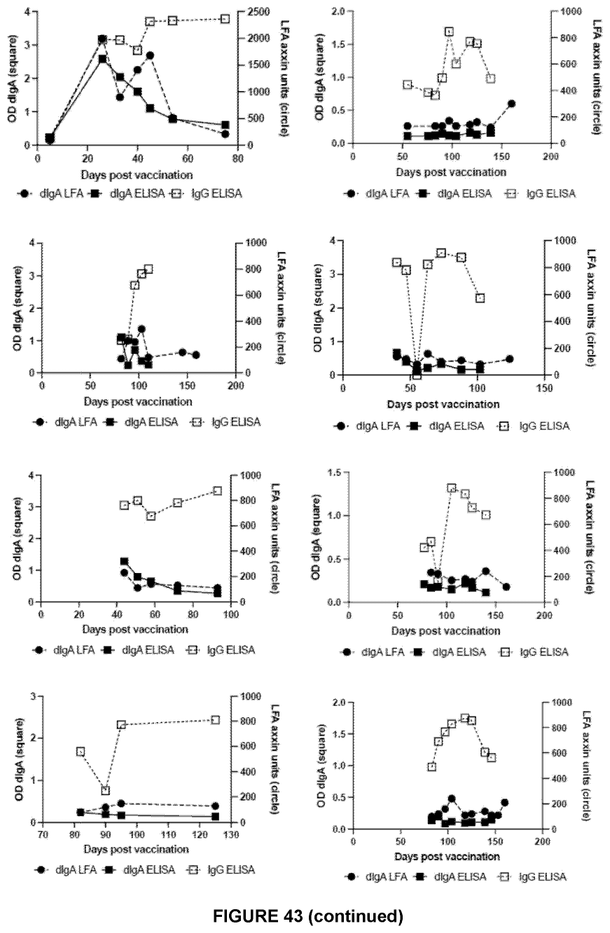

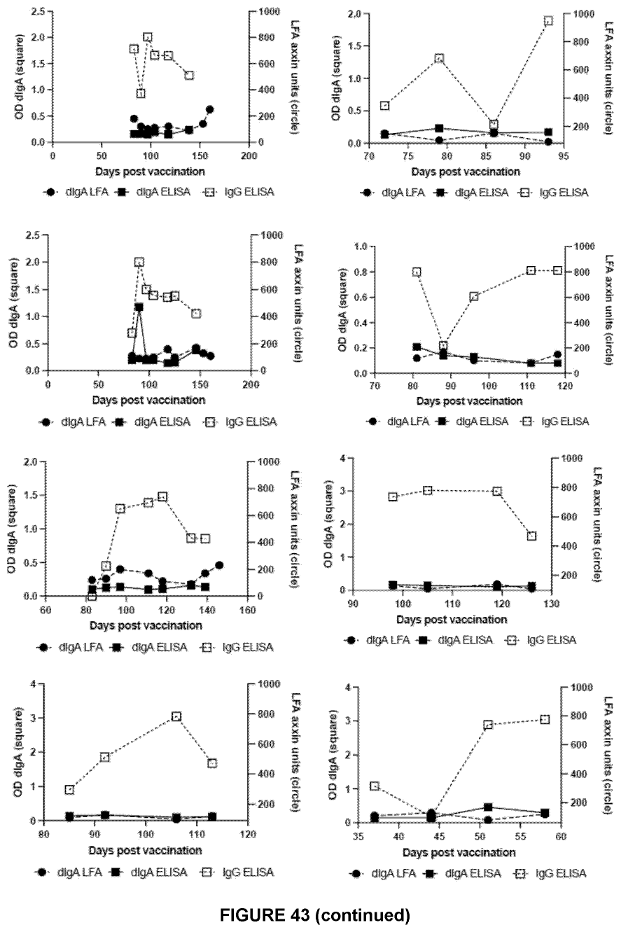

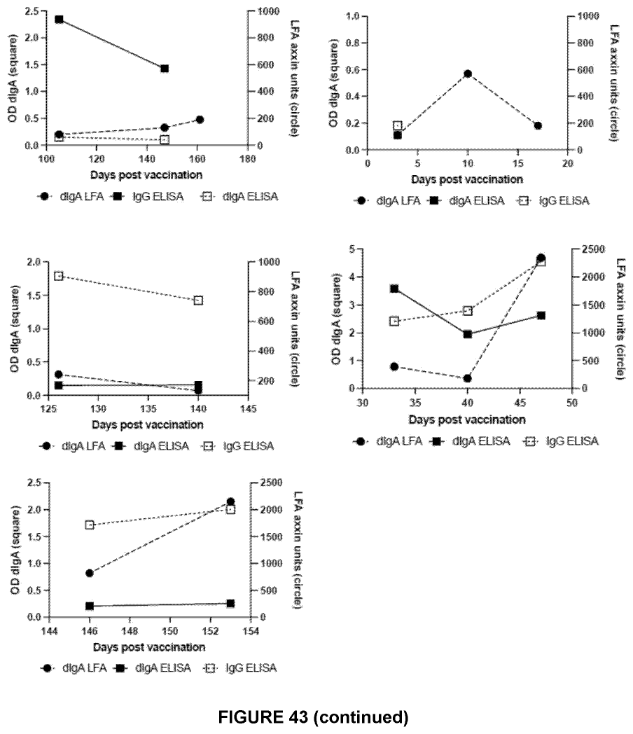

As shown in FIG. 43 where each graph represents an individual's immune response after vaccination. The presence of SARS-COV-2 receptor binding domain specific dimeric IgA in plasma was monitored either using a lateral flow assay (LFA) for dimeric IgA (circles), or an ELISA assay for the detection of dimeric IgA (solid squares), or an ELISA assay for the presence of SARS-COV-2 receptor binding domain specific IgG (open squares) as a measure of seroconversion to vaccine overall. The results show that a majority of individuals (61.3%) do not produce dimeric IgA following vaccination. This contrasts to data shown in FIG. 34A where 100% of samples tested positive for dIgA at day 11 post infection. The cut-off for dimeric IgA in LFA is 400 axxin units meaning any value of 400 or above is considered positive for dimeric IgA to SARS-COV-2. The cut-off for dimeric IgA in ELISA is 0.25 meaning that any value above 0.25 is positive for dimeric IgA to SARS-COV-2. The cut off for IgG is 0.5 meaning that any value above 0.5 is considered positive for the presence of IgG to SARS-COV-2.

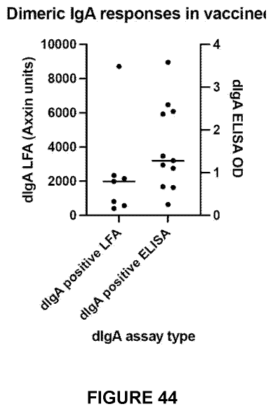

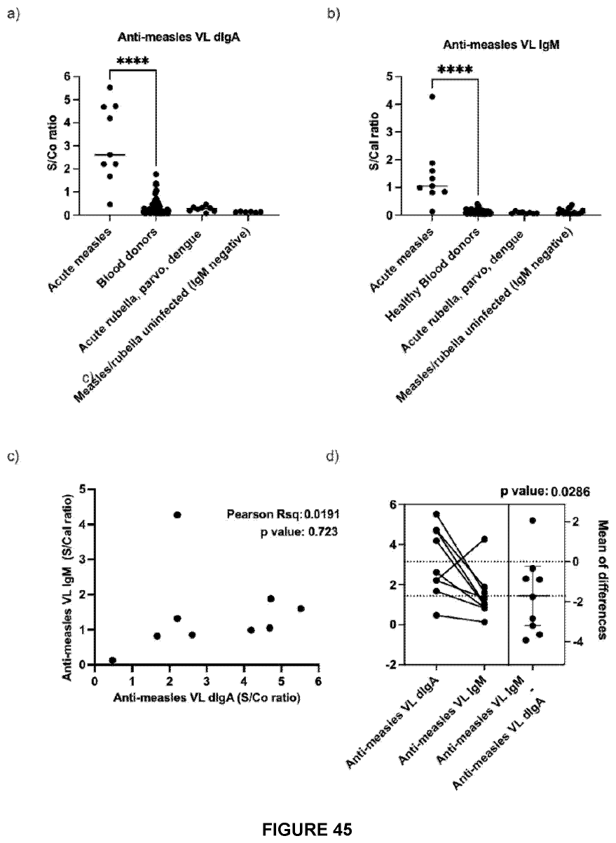

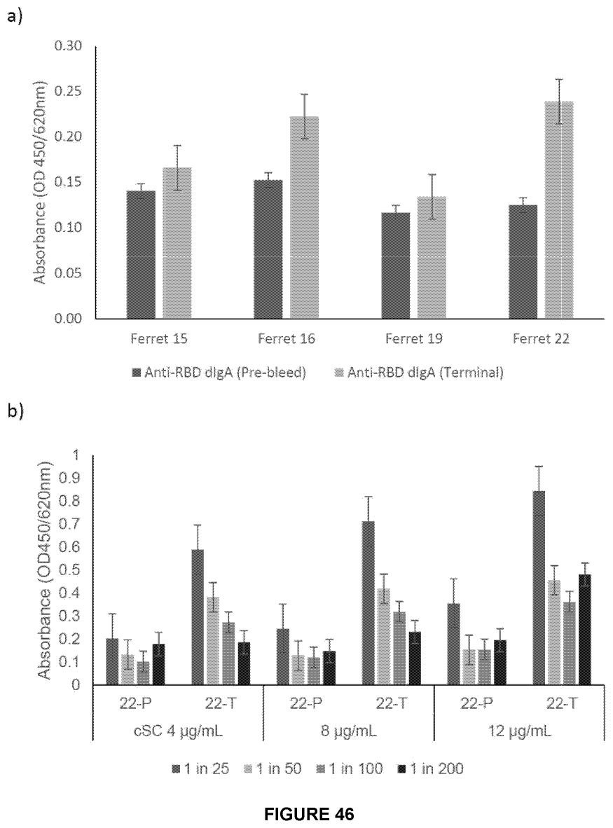

FIG. 44, shows the maximal levels of dIgA detected in those vaccine recipients who demonstrated a detectable dIgA response in either an LFA (n=7) or ELISA assay (n=11) among the 31 people that received SARS-COV-2 vaccines. The range of responses as measured in LFA was between 400 and 8710 units with a median of 1990 units. The range of dIgA levels as measured in ELISA was between 0.25 and 3.6 with a median of 1.3.

Table 5 tabulates results showing the percentage of individuals who developed a dIgA response post vaccination. Only 1 individual returned a positive dIgA result that was detectable in lateral flow only (3%). Five people returned a positive dIgA result that was only detectable in the dIgA ELISA (16%). Six people retuned a positive dIgA result detectable in both the lateral flow tests and the ELISA (19%). Altogether 12 people had detectable dIgA at any time after vaccination in either lateral flow assay or ELISA representing 38.7% of the cohort. This means 61.3% of vaccinated people did not make detectable dIgA at any time after vaccination.

In vaccines that received either an mRNA vaccine or a viral vectored vaccine encoding the SARS-COV-2 spike, it was not anticipated that subjects would develop dIgA responses as vaccines are administered intramuscularly. However, this analysis of 31 vaccines shows that while a majority do not develop dIgA (61.3%), a subset of vaccines do develop dIgA within 90 days of vaccination. It is known in the art that dIgA is actively transported across mucosal surfaces by the pIgR and so it is highly likely that vaccines who make pathogen specific dIgA including but not limited to neutralization targets (such as the spike protein) will have better mucosal immunity and therefore will be less likely to become infected with SARS-COV-2. Ongoing monitoring of these subjects in an ongoing outbreak of SARS-COV-2 will reveal the level of protection in the dIgA positive versus dIgA negative groups of vaccines.

dIgA responses in vaccines were observed within 90 days of vaccination (range 10-90 days, median 42 days after first vaccination). This suggests that as a combined antigen plus dIgA test for COVID-19, the test could only accurately determine recent infection where subjects were not vaccinated within the last 90 days. This is unlikely to be relevant as vaccine immunity is known to last at least 6 months post vaccination so people are unlikely to become COVID-19 positive within 3 months after receiving their first dose of vaccine. Therefore, the combination of antigen testing plus dIgA testing should provide a useful mechanism to detect recent COVID-19 infection and for backward contact tracing, even where vaccines are extensively used.

A comparison of the proportion of vaccines receiving either an adenoviral vaccine or an mRNA vaccine that generated a dIgA response reveals that mRNA vaccines were more likely to generate dIgA responses (p=0.0007). The implications for this are two-fold. For the use of dIgA, it is more likely to be able to discriminate infection versus vaccination in subjects that receive the adenoviral vaccine, even within three months of receiving a vaccine. Secondly, people receiving mRNA vaccines may have better mucosal protection against SARS-COV-2 infection. The efficacy of mRNA vaccines against symptomatic infection is >95% whereas the efficacy of adenoviral vaccines at preventing symptomatic infection is 55-82% depending on the spacing between first and second doses. This may be a result of the superior ability of mRNA vaccines to generate dIgA responses that is translated into mucosal immunity against SARS-COV-2. Recent data from a study of Pfizer (BNT162b2) vaccine recipients also shows that protection from any SARS-COV-2 infection declined from 88% (95% CI 86-89) during the first month after full vaccination to 47% (43-51) after 5 months, even though the protection from hospital admissions remains high (93% [95% CI 84-96]) up to 6 months. (Tartof, 2021). These results are consistent with transient mucosal protection against infection provided by low levels of dIgA and product SIgA in the first month after full immunisation with BNT162b2, compared to more durable protection from severe disease provided by high levels of IgG in the plasma. Table 6 tabulates results of the post vaccination studies showing the proportion of subjects that tested dIgA positive after vaccination with an adenoviral vaccine and an mRNA vaccine. Overall, 19% of people that received adenoviral vaccine became dIgA positive while 80% of those that received an mRNA vaccine became dIgA positive. This suggests that mRNA vaccines generate more dIgA responses than adenoviral vaccines and is significantly different (p=0.0007). The present kits, methods and assays will find broad application in screening vaccines by screening vaccinated subjects for their ability to seroconvert to produce a more durable mucosal protection from transmission or infection.

### Example 20

The CSC reagent can be used to detect dIgA in other respiratory infections such as measles. In this case, measles specific antibodies are bound to measles virus lysate and dIgA specific to the virus lysate detected using CSC reagent. This is an illustration of the utility of CSC to detect mucosal immune responses via pathogen specific dIgA from complex mixtures in response to recent infection with respiratory viruses more generally, extending its utility for detection of current (acute) infection with hepatitis viruses (Hanafiah et al 2018) and human immunodeficiency virus (Seaton et al 2017).

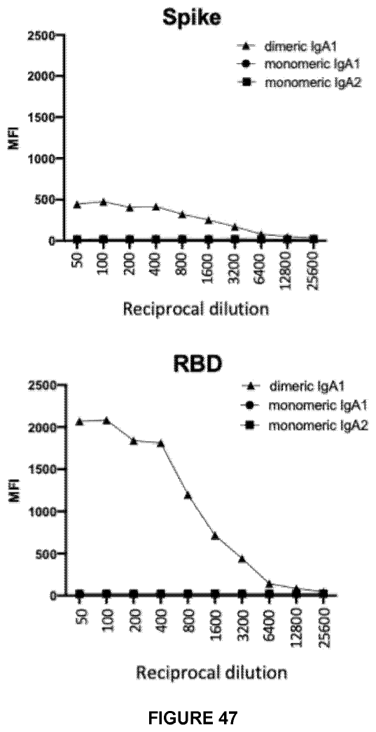

FIG. 47 (a) shows anti-measles virus lysate (VL) dIgA is detectable in samples with confirmed acute measles infection and is comparable to the gold standard IgM (b) with no detectable cross-reactivity in acute rubella, parvo or dengue samples and very low background reactivity from negative controls (samples with provisional diagnosis of measles/rubella but confirmed IgM negative and blood donors). Anti-measles VL dIgA has low correlation to IgM response (c), suggesting it is an independent biomarker with potential for higher signal-to-cut off than anti-measles VL IgM, as shown in pairwise comparison of dIgA and IgM and mean of difference plot for the majority of acute measles samples (d). ****, p value of <0.0001.

### Example 22

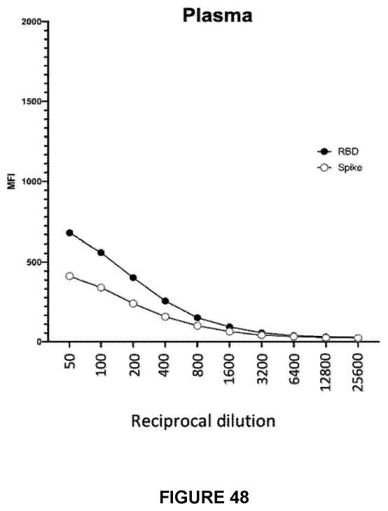

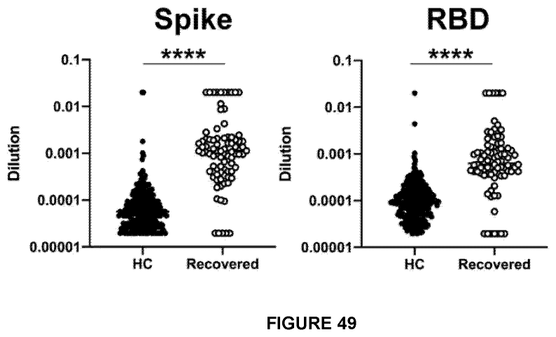

The CSC reagent has proven to be a highly sensitive reagent for detecting dIgA from within complex biological samples and the subject assays can be used to detect mucosal immune responses via levels of pathogen-specific dIgA in species other than humans. Ferrets experimentally infected with SARS-COV-2 produce receptor binding domain specific dIgA in response to infection. FIG. 48 (a) shows that RBD-specific dIgA is elevated in serum samples of two out of four ferrets experimentally infected with SARS-COV-2 (VIC01, 4.64×104 TCID50) and humanely killed 14 days after intranasal inoculation (Terminal bleed =T). Serum was collected from each ferret prior to challenge (Prebleed=P). FIG. 49 (b) shows dIgA in ferret serum captured using various concentrations of CSC reagent (4, 8, and 12 μg/mL) from various dilutions of serum (1 in 25, 1 in 50, 1 in 100 and 1 in 200). Ferret 22 further confirms the elevated presence of RBD-specific dIgA in the terminal bleed (22-T) sample compared to the pre-bleed (22-P) sample. Mean±standard error plotted using microsoft excel.

This shows the utility of CSC for detection of mucosal immune responses that generate dIgA in experimental animal disease models of human virus infections, and by inference CSC could also be used for detection of pathogen-specific dIgA in wild or domestic animals that are naturally infected with corresponding animal viruses, including viruses that may have potential or demonstrated zoonotic transmission between animals and humans, or between humans and animals, or both.

Together the results illustrate that CSC may be used for detecting dIgA responses in animals infected with other pathogens to provide a general method of detecting recent infection (even in the absence of a current (acute) infection). Examples where this approach may be useful include monitoring of immune responses to vaccines in animals to determine the ability to generate mucosal immunity, and similarly vaccine mucosal immunity (neutralising/transmission blocking responses) responses in humans especially those aimed at generating strong mucosal immunity.

Other potential uses include the use of CSC to detect recent infection with pathogens in animals such as emerging pandemic viruses. Surveying animals for the presence of dIgA towards an emerging pandemic virus would allow recent infections to be diagnosed allowing tracing of infections. An example would be wet markets where many different animal species are housed in close proximity. A further example would be commercial farming of animals with known susceptibility to circulating pandemic viruses, such as Mustelidae (mink and related animals) which have been a vector for SARS-COV-2 with severe economic losses due to culling of infected and exposed animals, as well as further transmission to human handlers. Sero-surveys for the presence of pathogen specific dIgA would allow recent infections to be traced providing valuable information about the spread of pathogens in different species and potential transmission contact points between animals and humans to be revealed.

### Example 23

The CSC reagent for the specific detection of dIgA can be used in many different platforms including ELISA-based assay, lateral or reverse flow, luminex (fluorescent bead assays) and other assays known to those skilled in the art.

In a Luminex bead based assay-Polyclonal and monoclonal antibodies were diluted in PBS containing 1% BSA 0.05% tween 20 (PBT) and added to an equal volume of CSC at 4 ug/ml in PBT prior to use. Samples were incubated at ambient temperature for 2 hours then 25 ul of antigen-bead mix (RBD or Spike protein) was added to each well. Samples were incubated for 60 min and then washed in PBT. To detect bound dIgA, 50 ul of monoclonal anti-SC antibody was added at 1:2000 dilution in PBT to each well and incubated for 30 min. Following incubation, wells were washed and 50 ul of goat anti mouse-PE diluted 1:100 (Sigma) added to each well. Following further incubation (30 min) wells were washed and resuspended in 100 ul of PBT before analysing on Magpix machine.

FIG. 47 shows an example of the adaptation of the dIgA assay to a fluorescent bead platform for the detection of dimeric IgA. The assay sensitivity (LHS) and specificity for the detection of dIgA (RHS) is shown using monoclonal dimeric IgA1 (9 mg/ml) and monomeric IgA1 (4.5 mg/ml) and monomeric IgA2 (2 mg/ml). Monomeric IgA does not bind CSC confirming the specificity of the assay towards only dimeric IgA. The sensitivity of the assay using spike protein is 2.8 μg/ml. The assay can detect dIgA specific to any antigen as evidenced through the use of Spike and receptor binding domain (RBD) antigens here. Monoclonal antibodies specific for either dIgA1, monomeric IgA1 or monomeric IgA2 is used to validate that this assay only detects RBD- and Spike-specific dIgA and not monomeric IgA. This is demonstrated by a detectable signal in the serial dilutions containing dimeric IgA1 down to 2.8 μg/ml and absence of a signal with monomeric IgA1 or monomeric IgA2-specific monoclonal antibodies.

FIG. 48 shows an example of the use a bead-based dIgA Luminex® assay (as above) that specifically detects dIgA against SARS-COV-2 antigens in plasma allows measurement of the production of dimeric IgA following infection with SARS-COV-2 or vaccination with SARS-COV-2 antigens. The levels of dIgA antibodies towards the SARS-CoV-2 antigens RBD and Spike are presented as the median fluorescence intensity (MFI) values detected in serial dilutions prepared from a pool (n=10) of plasma from confirmed SARS-COV-2 convalescent individuals. FIG. 48 shows a dose dependent reduction in signal intensity against RBD and Spike-specific dIgA in human plasma using the dIgA Luminex assay.

FIG. 49 shows the utilisation of the dIgA Luminex assay in measuring dIgA responses towards SARS-COV-2 antigens Spike and RBD in both healthy control (HC) individuals (n=252) and convalescent plasma from individuals with confirmed SARS-COV-2 infection (recovered) (n=84). Cross-sectional samples were obtained following confirmed SARS-COV-2 infection and analysed in the bead-based assay for the presence of either Spike specific (LHS) or receptor binding domain (RBD) specific (RHS) dIgA, represented as the highest terminal dilution that shows a positive signal above background. The data show a significant increase in the levels of dIgA following SARS-COV-2 infection compared to the levels present in healthy control subjects. Mann-Whitney t test, p<0.0001.

### Example 24

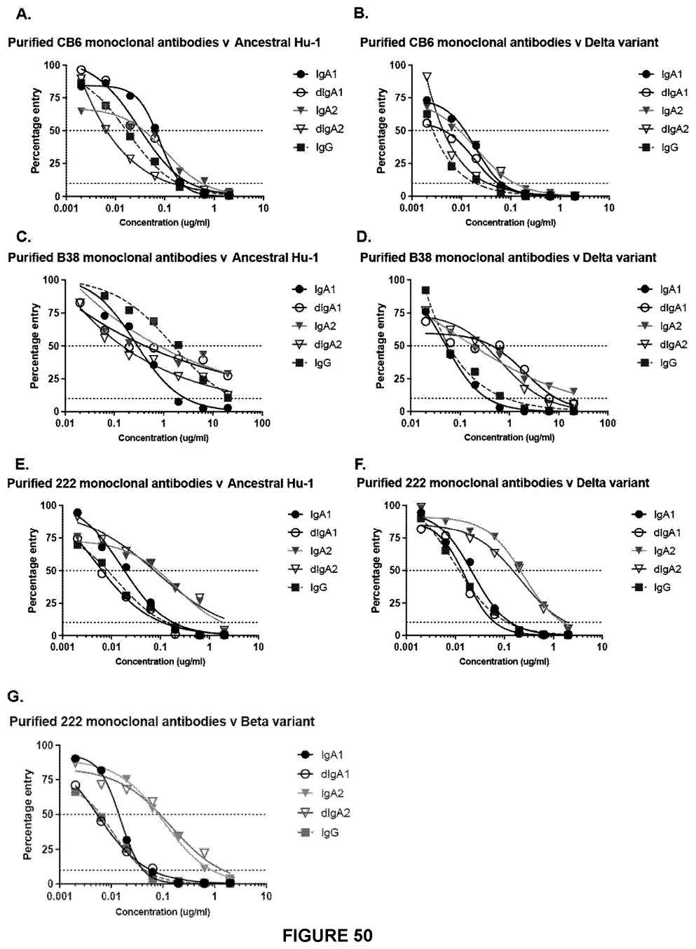

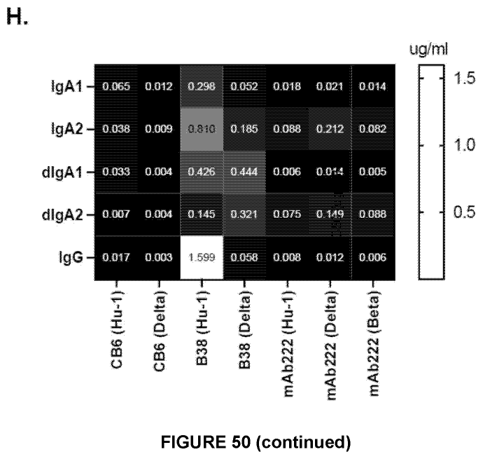

FIG. 50 shows examples where monoclonal antibodies originally isolated and characterised as IgG isotypes CB6 B38 and MAb 222 (Shi et al. (2020); Wu et al. (2020); Dejnirattisai et al. (2021)) have been converted into IgA1 or IgA2 isotypes and expressed as either monomeric IgA1 or IgA2, or expressed as dimeric IgA1 and IgA2. The ability of the antibodies to prevent entry of pseudotyped viruses bearing spike proteins from variants of concern from entering susceptible cells was measured. The data shows that all of the tested antibodies retain activity when converted into either IgA1, IgA2, dIgA1 and dIgA2 isotypes against the ancestral Hu-1 strain, as well as variants of concern including the globally prevalent Delta strain. In the case of MAb222 which is a broadly neutralizing antibody, reactivity was also retained against the highly neutralization resistant Beta variant (Ho et al. (2021)). In the case of CB6, conversion into dIgA2 resulted in a 2.5 fold improvement compared with IgG version in IC50 against the Hu-1 isolate (A) and a similar level of reactivity of IgG against the Delta variant (B). In the case of B38, conversion into IgA or dimeric IgA forms improved the activity of this MAb against the ancestral Hu-1 isolate 2-10 fold relative to IgG form (C). In the case of broadly reactive MAb 222, dIgA1 retained a similar level of activity against the Hu-1 isolate (E) the delta isolate (F) and the Beta isolate (G) compared to IgG. These examples show how an IgG can be converted to IgA or dimeric IgA and retain or improve activity both ancestral and variants of SARS-COV-2. The IC50 data is summarised in a heat map in (H). The lower the value the more potent the antibody activity and is shown in ug/ml.

Monoclonal antibodies have proven to be an effective therapy against SARS-COV-2 infection when given at high concentrations intravenously early after exposure to virus (Kumar et al, 2021 incorporated herein in its entirety). Many potent neutralizing antibodies have been discovered that target epitopes located in the RBD, S1 domain (Outside of the RBD) or within the N-terminal domain of the spike protein. Some have limited cross-neutralization capacity as their epitopes are altered in variants of concern, while others target more conserved regions that are preserved in variants of concern. While IgG therapies are given intravenously and rely on passive transfer across mucosal surfaces, it is proposed that dimeric IgA provide a distinct advantage that it is actively transported via the polymeric Ig receptor. As determined herein, an isolated IgG antibody can be converted into either monomeric IgA1 or IgA2 or the dimeric IgA1 or dimeric IgA2 forms and retains the ability to prevent entry (neutralize) of SARS-COV-2. It is proposed that the use of a composition comprising dIgA1 or dIgA2 or a cocktail of both instread of IgG, would allow significantly smaller amounts of antibody to be administered into patients to achieve the same or better therapeutic effect than the use of IgG. In addition, its is shown herein that the activity of some IgGs can be improved by conversion into dimeric IgA forms further demonstrating a benefit to the use of dIgA over IgG isotypes as a therapeutic option for SARS-COV-2.

TABLE 2
Amino acid sub-classification

| Sub-classes | | Amino acids | |
|---|---|---|---|
| Acidic | | Aspartic acid, Glutamic acid | |
| Basic | | Noncyclic: Arginine, Lysine; Cyclic: Histidine | |
| Charged | | Aspartic acid, Glutamic acid, Arginine, Lysine, Histidine | |
| Small | | Glycine, Serine, Alanine, Threonine, Proline | |
| Polar/neutral | | Asparagine, Histidine, Glutamine, Cysteine, Serine, Threonine | |
| Polar/large | | Asparagine, Glutamine | |
| Hydrophobic | | Tyrosine, Valine, Isoleucine, Leucine, Methionine, | |
| | | Phenylalanine, Tryptophan | |
| Aromatic | | Tryptophan, Tyrosine, Phenylalanine | |
| Residues that influence | | Glycine and Proline | |
| chain orientation | | | |
| | Original residue | Exemplary substitutions | Preferred substitutions |
| | Ala | Val, Leu, Ile | Val |
| | Arg | Lys, Gln, Asn | Lys |
| | Asn | Gln, His, Lys, Arg | Gln |
| | Asp | Glu | Glu |
| | Cys | Ser | Ser |
| | Gln | Asn, His, Lys, | Asr |
| | Glu | Asp, Lys | Asp |
| | Gly | Pro | Pro |
| | His | Asn, Gln, Lys, Arg | Arg |
| | Ile | Leu, Val, Met, Ala, Phe, Norleu | Leu |
| | Leu | Norleu, Ile, Val, Met, Ala, Phe | Ile |
| | Lys | Arg, Gln, Asn | Arg |
| | Met | Leu, Ile, Phe | Leu |
| | Phe | Leu, Val, Ile, Ala | Leu |
| | Prc | Gly | Gly |
| | Ser | Thr | Thr |
| | Th | Ser | Ser |
| | Trp | Tyr | Tyr |
| | Tyr | Trp, Phe, Thr, Ser | Phe |
| | Val | Ile, Leu, Met, Phe, Ala, Norleu | Leu |

TABLE 3
Detection of SARS-CoV-2 specific antibody
isotypes days post onset of symptoms.

| | | | | Calculated days |
|---|---|---|---|---|
| | | | | from symptom |
| Antibody | Days post symptom onset (%)² | | | onset to earliest |
| type¹ | 0-5 | 6-10 | 11-15 | 100% positive³ |
| dIgA | 49 | 65 | 100 | 11 |
| IgM | 0 | 39 | 92 | 13 |
| IgG | 0 | 34 | 67 | 14 |

¹Plasma samples were analysed in the dIgA LFA (dIgA), EDI IgM ELISA (IgM), EDI IgG ELISA (IgG).
²The percentage of samples tested that recorded a positive result
³Number of days post symptom onset before 100% of samples tested positive.

TABLE 4
Characteristics of the 22 non-SARS-CoV-2 coronavirus panel

| Sample | EDI-IgM | Rhinovirus/ | | PCR result | | | | Para- | IgG result | | | | | |
|---|---|---|---|---|---|---|---|---|---|---|---|---|---|---|
| ID | Result | Enterovirus | Influenza | 229E | HKU1 | NL63 | OC43 | influenza | HIV | 229E | HKU1 | NL63 | OC43 | dIgA |
| 86 | − | + | − | − | − | − | − | − | − | + | + | + | + | + |
| 85 | − | + | − | − | − | − | − | − | − | + | + | + | + | − |
| 69 | − | + | − | − | − | − | − | − | − | − | + | + | + | − |
| 68 | − | + | − | − | − | − | − | − | − | − | + | + | + | − |
| 67 | − | + | − | − | − | − | − | − | − | − | + | + | + | − |
| 93 | − | + | − | − | − | − | − | − | − | + | + | − | − | − |
| 73 | − | + | − | − | − | − | − | + | + | + | + | + | + | − |
| 82 | + | − | − | + | − | − | − | − | − | + | + | + | + | − |
| 44 | − | − | − | − | − | + | − | − | + | + | + | + | + | − |
| 95 | − | + | − | − | − | − | − | − | − | + | + | + | + | − |
| 84 | − | + | − | − | − | − | − | − | − | + | + | + | + | − |
| 83 | − | + | − | − | − | − | − | − | + | + | + | + | + | − |
| 57 | − | + | − | − | − | − | − | − | − | + | + | + | + | − |
| 60 | − | + | − | − | − | − | − | − | − | − | + | − | − | − |
| 100 | − | + | − | − | − | − | − | − | − | + | − | + | + | − |
| 58 | − | + | − | − | − | − | − | − | − | − | + | + | − | − |
| 80 | − | − | − | − | − | − | + | − | + | + | + | + | + | − |
| 81 | − | − | − | − | − | − | + | − | − | + | + | + | + | − |
| 55 | − | + | − | − | − | − | − | − | − | − | + | + | − | − |
| 91 | − | + | − | − | − | − | − | − | − | + | + | + | + | − |
| 61 | − | + | − | − | − | − | − | − | − | + | + | + | + | − |
| 98 | − | + | − | − | − | − | − | − | + | + | + | + | + | − |

TABLE 5
Percentage of individuals who developed
a dIgA response post vaccination

| | Number dIgA | Percentage dIgA |
|---|---|---|
| dIgA result | positive | positive |
| Positive in lateral flow only | 1 | 3.22 |
| Positive in ELISA only | 5 | 16 |
| Positive in lateral flow AND | 6 | 19 |
| ELISA | | |
| Total dIgA positive in any | 12 | 38.7 |
| test | | |
| Number negative in any test | 19 | 61.3 |

TABLE 6
Proportion of subjects that tested dIgA positive after vaccination
with an adenoviral vaccine and an mRNA vaccine

| | Adenoviral vaccine | mRNA vaccine |
|---|---|---|
| dIgA positive | 4 | 8 |
| Total number of subjects | 21 | 10 |
| Percentage dIgA positive | 19% | 80% |

### REFERENCES

Afonso & Joao, (2016) Biomolecules, 6: 15
Ausubel et al., Current Protocols in Molecular Biology, Supplement 47, John Wiley & Sons, New York, 1999
Braathen R, Sorensen V, Brandtzaeg P, Sandlie I, Johansen F E. The carboxyl-terminal domains of IgA and IgM direct isotype-specific polym-erization and interaction with the polymeric immunoglobulin receptor. J Biol Chem. 2002; 277:42755-62
Bundschuh, C. et al. Evaluation of the EDI enzyme linked immunosorbent assays for the detection of SARS-COV-2 IgM and IgG antibodies in human plasma. Clin Chim Acta 509, 79-82 (2020).
Cahill (2000) Trends in Biotechnology 7:47-51
Castro and Flajnik, (2014) J Immunol. 193(7): 3248-3255
Cohen et al. (1998) Proc. Natl. Acad. Sci. USA 95:14272-14277
Colowick and Kaplan, eds., Methods In Enzymology, Academic Press, Inc.
Dan, J. M. et al. Immunological memory to SARS-COV-2 assessed for up to 8 months after infection. Science 371 (2021).
Deeks, J. J. et al. Antibody tests for identification of current and past infection with SARS-COV-2. Cochrane Database Syst Rev 6, CD013652 (2020). Dejnirattisai, W. et al; Antibody evasion by the P.1 strain of SARS-COV-2. Cell 2021, 184, (11), 2939-2954 e9.
Dinnes, J. et al. Rapid, point-of-care antigen and molecular-based tests for diagnosis of SARS-CoV-2 infection. Cochrane Database Syst Rev 3, CD013705 (2021).
Foster et al., (2020), PNAS 117(17) 9241-9243
Fukuda, et al. (1997) Nucleic Acids Symp. Ser. 37:237-238
Ge (2000), Nucleic Acids Res. 28(2): e3
Gordon et al., (2020), bioRxiv doi: 10.1101/2020.03.22.002386
Harlow and Lane (1988), Antibodies. A Laboratory Manual, Cold Spring Harbor Publications, New York
Ho, D et al., Increased Resistance of SARS-COV-2 Variants B.1.351 and B.1.1.7 to Antibody Neutralization. Res Sq 2021.
Houlihan, C. F. & Beale, R. The complexities of SARS-COV-2 serology. Lancet Infect Dis 20, 1350-1351 (2020).
Iacobelli et al., (1988) Breast Cancer Research and Treatment 11:19-30
Jackson et al., An mRNA Vaccine against SARS-COV-2-Preliminary Report (2020), NEJM, DOI: 10.1056/NEJMoa2022483)
Johansen F E, Kaetzel C S. Regulation of the polymeric immunoglobulin receptor and IgA transport: new advances in environmental factors that stimulate pIgR expression and its role in mucosal immunity. Mucosal Immunol. 2011; 4:598-602
Khan et al., (2020), PloS One 3; 15(9):e0238344. DOI: 10.1371/journal.pone.0238344.
Klein, S. L. et al. Sex, age, and hospitalization drive antibody responses in a COVID-19 convalescent plasma donor population. J Clin Invest 130, 6141-6150 (2020).
Kucirka et al, Ann Intern Med. doi:10.7326/M20-1495
Kumar, S.; Chandele, A.; Sharma, A., Current status of therapeutic monoclonal antibodies against SARS-COV-2. PLOS Pathog 2021, 17, (9), e1009885
Landry, M. L. Immunoglobulin M for Acute Infection: True or False? Clin Vaccine Immunol 23, 540-545 (2016).
Long, Q. X. et al. Clinical and immunological assessment of asymptomatic SARS-COV-2 infections. Nat Med 26, 1200-1204 (2020).
Lopez et al., (2003), J. Chromatogr. B 787(1): 19-27
Mohd Hanafiah et al., (2018) Detection of virus-specific polymeric immunoglobulin A in acute hepatitis A, C, E virus serum samples using novel chimeric secretory component. BMC Res Notes 11, 688 (2018).
Muruato et al., (2020), Nature Communications 11: Article number 4059
National SARS-COV-2 Serology Assa Evaluation Group (2020). Performance characteristics of five immunoassays for SARS-COV-2: a head-to-head benchmark comparison. Lancet Infect Dis 20, 1390-1400
Phan et al., (2020) Infection, Genetics and Evolution 81:104260. DOI: 10.1016/j.meegid.2020.104260
Potyrailo et al., (1998), Anal. Chem. 70:3419-3425
Remington's Pharmaceutical Sciences, 1990
Roberts and Szostak (1997), Proc. Natl. Acad. Sci. USA 94(23): 12297-12302
Roe M, Norderhaug I N, Brandtzaeg P, Johansen F E. Fine specificity of ligand-binding domain 1 in the polymeric Ig receptor: importance of the CDR2-containing region for IgM interaction. J Immunol. 1999; 162:6046-52
Shen et al. (2020), Clin. Infect. Dis. 71(15) 713-720
Shi et al. (2020) A human neutralizing antibody targets the receptor-binding site of SARS-COV-2. Nature 584, 120-124
Sousa-Pereira et al., (2019) Antibodies 8(4): 57
Steenhuis, M. et al. (2021) Dynamics of antibodies to SARS-COV-2 in convalescent plasma donors. medRxiv (2021).
Tang et al., (2020) National Science Review 7(6): 1012-1023
Sara Y Tartof, Jeff M Slezak, Heidi Fischer, Vennis Hong, Bradley K Ackerson, Omesh N Ranasinghe, Timothy B Frankland, Oluwaseye A Ogun, Joann M Zamparo, Sharon Gray, Srinivas R Valluri, Kaije Pan, Frederick J Angulo, Luis Jodar, John M Mclaughlin Effectiveness of mRNA BNT162b2 COVID-19 vaccine up to 6 months in a large integrated health system in the USA: a retrospective cohort study. Published Online Oct. 4, 2021 https://doi.org/10.1016/S0140-6736(21)02183-8.
Wang et al. Sci. Transl. Med. 10.1126/scitranslmed.abf1555 (2020)
Watson, J. D. et al., “Molecular Biology of the Gene”, Fourth Edition, Benjamin/Cummings, Menlo Park, California, 1987
Watson, J., Richter, A. & Deeks, J., Testing for SARS-COV-2 antibodies. BMJ 370, m3325 (2020).
Weir and Blackwell, eds., Handbook of Experimental Immunology, Vols. I-IV, Blackwell Scientific Publications, 1986
Wild D., “The Immunoassay Handbook”. Nature Publishing Group, 4th Edition, 2013
William E Paul, ed, Fundamental Immunology Fifth-Seventh Edition 2003-present
Woof and Russel, 2011 Mucosal Immunity November; 4(6):590-7, doi: 10.1038/mi.2011.39. Epub 2011 Sep. 21
Wu, Y.; et al., A noncompeting pair of human neutralizing antibodies block COVID-19 virus binding to its receptor ACE2. Science 2020, 368, (6496), 1274-1278)
Xie et al., (2013), Clin. Vaccine Immunology 20(4):517-25.

1-33. (canceled)

34. A method for assessing the mucosal or transmission blocking immune response in a subject or population to infection by a viral mucosal pathogen, vaccination against a pathogen, or to post-vaccination challenge, the method comprising performing an assay to determine the pathogen-specific dIgA antibody level in a biological sample from a subject according to any one of the preceding claims, wherein the level of pathogen specific dIgA positively correlates with the magnitude of a transmission blocking mucosal immune response in the subject.

35. The method of claim 34, comprising determining the area under the curve (AUC) of dIgA level over time to assess the total dIgA response of the subject over time in response to the pathogen or vaccination (including post vaccination challenge) and therefore predict the total level of the mucosal immune response produced by the subject.

36. The method kit or use of claim 35, wherein the subject is a human, mammal or reservoir host subject.

37. An in vitro method of identifying or stratifying a subject convalescing after an infection by a pathogen, said method comprising assessing or measuring the level of pathogen specific dimeric IgA (dIgA) in a biological sample from the convalescent subject, wherein the identification of the level of pathogen specific dIgA in the sample is positively correlated with and indicates the production by the subject of an effective (e.g., neutralising) pathogen specific immune response and the suitability of the subject for prioritisation as a blood or plasma donor for therapeutic or prophylactic convalescent plasma therapy, or as a source of dIgA expressing plasma cells or dIgA immunoglobulin there from, to be administered against the pathogen.

38. The method of claim 37, wherein the pathogen is a virus that is transmitted via a mucosal surface of the subject and/or the pathogen is a respiratory viral pathogen.

39. (canceled)

40. The method of claim 37, wherein the level or presence of pathogen specific dIgA is determined by performing an assay that employs a binding reagent that specifically binds dIgA and does not substantially bind monomeric IgA, IgG or IgM within a biological sample from the subject.

41. The method of claim 40 wherein the binding reagent is a pIgR based specific dIgA binding reagent, such as CSC.

42. The method of claim 41, wherein the pIgR-based reagent comprises at least domain 1 from a lagomorph, such as chimeric secretory component (CSC) which comprises human pIgR with domain 1 substituted with a rabbit domain 1.

43. The method of claim 37, wherein the pathogen specific dIgA comprises dIgA that is functionally active (e.g., neutralising) against the pathogen in vivo or in vitro.

44. The method of claim 37, wherein the assay comprises one or more of the following features:
i) the assay employs an antigen of the pathogen that is the target of neutralising or non-neutralising dIgA;
ii) the assay comprises contacting the biological sample with the pIgR based dIgA binding reagent and the antigen of the pathogen to form a directly or indirectly detectable complex comprising the pIgR based dIgA binding agent and the pathogen specific dIgA;
iii) the assay employs ECLIA, IFA, ELISA-type, flow cytometry, interferometry methods, bead array, lateral flow, cartridge, microfluidic, förster resonance energy transfer, immunochromatographic based methods, nucleic acid based methods; and
iv) the assay is conducted using a lateral or reverse flow device or microfluidic device suitable for point of care;
v) the pathogen specific dIgA is detected using a quantifiable binding pair or label such as a fluorescent or chemiluminescent label;
vi) enriching or purifying pathogen specific dIgA from a blood or plasma sample from the subject, wherein the dIgA is enriched or purified using a pIgR based dIgA binding reagent, such as CSC; and
vii) isolating IgA positive plasma cells from the subject, furthermore wherein the dIgA expressing plasma cells are isolated or identified using a pIgR based dIgA binding reagent, such as CSC.

45-51. (canceled)

52. The method of claim 37, wherein the biological sample is whole blood or a fraction thereof, a blood product, plasma, serum; a mucosal sample such as saliva, nasal swab, throat swab, respiratory swab, nasal scrapings, nasal washings, respiratory tract washing, lung washings, gut samples, faeces or gingiva creviscular fluid.

53. The method of claim 37, wherein the antigen is a viral surface protein.

54. The method of claim 37, wherein the pathogen is a coronavirus and the antigen is a spike protein or wherein the pathogen is a coronavirus and the level of a capsid protein antigen is detected.

55-56. (canceled)

57. The method of claim 37, wherein the subject is a human, mammal or reservoir host subject.

58. The method of claim 37, wherein the assessment further comprises confirming the subject is negative for the pathogen.

59-64. (canceled)

65. A method of treatment or prophylaxis comprising administering an effective amount of a composition, comprising as an active agent a pathogen specific dIgA antibody, or an antigen binding derivative thereof, and a pharmaceutically acceptable carrier or diluent, wherein the antibody or derivative directly or indirectly provides coronavirus transmission blocking activity.

66. The method of claim 65, wherein the composition is or comprises one or more of:
i) a coronavirus specific polyclonal dIgA antibody that binds a Spike protein antigen, or an antigen binding derivative thereof;
ii) a coronavirus specific dIgA monoclonal antibody that binds a Spike protein antigen, or an antigen binding derivative thereof; and
iii) a coronavirus specific dIgA antibody that binds a Spike protein, wherein the antibody is derived from a subject exposed to the coronavirus, or an antigen binding derivative thereof.

67. The method of claim 65, wherein the antibody is a human antibody or humanised antibody.

68. The method of claim 65, wherein the antibody comprises the sequences set out in one or more of SEQ ID NO: 21 to 24 or a sequence having at least 98% sequence identity thereto, or comprising 1-3 conservative substitutions.

69-104. (canceled)

105. The method of claim 65, wherein the composition is or comprises a coronavirus specific dIgA antibody, or an antigen biding derivative thereof.
